(19) United States
(12) Patent Application Publication
(10) Pub. No.: US 2026/0248528 A1
(43) Pub. Date: Aug. 27, 2026

(54) Ultrasonic Surgical System

(71) Applicant: Stryker Corporation, Portage, MI (US)

(72) Inventor: Adam D. Downey, Kalamazoo, MI (US)

(73) Assignee: Stryker Corporation, Portage, MI (US)

(21) Appl. No.: 18/854,512

(22) PCT Filed: Apr. 6, 2023
(86) PCT No.: PCT/US2023/017790

§ 371 Date: Oct. 4, 2024

Related U.S. Application Data
(60) Provisional application No. 63/362,599, filed on Apr. 6, 2022.
(60) Provisional application No. 63/362,598, filed on Apr. 6, 2022.
(60) Provisional application No. 63/413,223, filed on Oct. 4, 2022.

(51) Int. Cl. A61B 17/32; A61B 17/00; A61B 34/10; A61B 34/20; G16H 40/63

(52) U.S. Cl. CPC A61B 17/320068; A61B 34/20; G16H 40/63; A61B 2017/00176; A61B 2017/320069; A61B 2017/320084; A61B 2034/107

(57) ABSTRACT

An ultrasonic instrument includes a tip and a driver coupled to the tip, the driver configured to vibrate the tip to ablate tissue at a target site responsive to receiving an AC drive signal from a power supply. A localizer is configured to generate localization data indicative of a pose of the ultrasonic instrument in a known coordinate system. A control system coupled to the power supply and the localizer is configured to, based on a received medical image including a tumorous region, generate a virtual boundary associated with the tumorous tissue region in the known coordinate system, track the pose of the ultrasonic instrument in the known coordinate system based on the localization data, and set the AC drive signal generated by the power supply to induce first pulsed ultrasonic energy in the tip based on the tracked pose of the ultrasonic instrument and virtual boundary.

### RELATED APPLICATIONS

This application claims priority to and all the benefits of U.S. Provisional Patent Application No. 63/362,598, filed on Apr. 6, 2022, U.S. Provisional Patent Application No. 63/362,599, filed on Apr. 6, 2022, and of U.S. Provisional Patent Application No. 63/413,223, filed on Oct. 4, 2022. The disclosure of each of these applications is hereby incorporated herein by reference in its entirety.

### BACKGROUND

Ultrasonic surgical instruments are often used to remove tissue from sites with limited visibility, which can make it difficult for a surgeon to distinguish between tissue at the site targeted for removal and surrounding tissues desired to remain intact. Such limited visibility can also make it difficult to determine whether the targeted tissue has been removed in its entirety.

### SUMMARY

In one aspect, an ultrasonic surgical system includes an ultrasonic instrument having a tip and a driver coupled to the tip, the driver configured to vibrate the tip to ablate tissue from a target site responsive to receiving an AC drive signal, a power supply coupled to the ultrasonic instrument and configured to generate the AC drive signal supplied to the driver, a localizer configured to generate localization data indicative of a pose of the ultrasonic instrument in a known coordinate system, and a control system coupled to the power supply and the localizer. The control system is configured to: receive a medical image of the target site that includes a tumorous tissue region; based on the medical image, generate a virtual boundary associated with the tumorous tissue region in the known coordinate system; based on the localization data, track the pose of the ultrasonic instrument in the known coordinate system; and based on the tracked pose of the ultrasonic instrument and virtual boundary, set the AC drive signal generated by the power supply to induce first pulsed ultrasonic energy in the tip.

In a further aspect, an ultrasonic surgical system includes an ultrasonic instrument having a tip and a driver coupled to the tip, the driver configured to vibrate the tip to ablate tissue from a target site responsive to receiving an AC drive signal, a power supply coupled to the ultrasonic instrument and configured to generate the AC drive signal supplied to the driver, a localizer configured to generate localization data indicative of a pose of the ultrasonic instrument in a known coordinate system, and a control system coupled to the power supply and the localizer. The control system is configured to: receive a medical image of the target site including a first tissue region to be ablated; based on the medical image, generate a virtual boundary associated with the first tissue region in the known coordinate system; based on the localization data, track the pose of the ultrasonic instrument in the known coordinate system; and based on the tracked pose of the ultrasonic instrument in the known coordinate system relative to the virtual boundary, set the AC drive signal generated by the power supply to induce first pulsed ultrasonic energy in the tip.

In a further aspect, an ultrasonic surgical system includes an ultrasonic instrument having a tip and a driver coupled to the tip, the driver configured to vibrate the tip to ablate tissue from a target site responsive to receiving an AC drive signal, a power supply coupled to the ultrasonic instrument and configured to generate the AC drive signal supplied to the driver, a localizer configured to generate localization data indicative of a pose of the ultrasonic instrument in a known coordinate system, and a control system coupled to the power supply and the localizer. The control system is configured to: receive a medical image of the target site that includes a soft tissue region and a hard tissue region; based on the medical image, generate a virtual boundary between the soft tissue and hard tissue regions in the known coordinate system; based on the localization data, track the pose of the ultrasonic instrument in the known coordinate system; based on the tracked pose of the ultrasonic instrument in the known coordinate system relative to the virtual boundary, determine whether the ultrasonic instrument is within the hard tissue region or the soft tissue region; responsive to determining that the ultrasonic instrument is within the soft tissue region, generate a first AC drive signal that induces first pulsed ultrasonic energy in the ultrasonic instrument, the first pulsed ultrasonic energy may include a plurality of first ultrasonic energy pulses interspaced by first periods of ultrasonic energy at a first minimum ultrasonic energy level, and each of the first ultrasonic energy pulses peaking at a maximum ultrasonic energy level set for the ultrasonic instrument for a second period that is less than each of the first periods; and responsive to determining that the ultrasonic instrument is within the hard tissue region, generate a second AC drive signal that induces second pulsed ultrasonic energy in the ultrasonic instrument, the second pulsed ultrasonic energy may include a plurality of second ultrasonic energy pulses interspaced by third periods of ultrasonic energy at a second minimum ultrasonic energy level, and each of the second ultrasonic energy pulses peaking at the maximum ultrasonic energy level for a fourth period that is greater than or equal to each of the third periods.

In a further aspect, an ultrasonic surgical system includes an ultrasonic instrument having a tip and a driver coupled to the tip, the driver configured to vibrate the tip to ablate tissue from a target site responsive to receiving an AC drive signal, a power supply coupled to the ultrasonic instrument and configured to generate the AC drive signal supplied to the driver, a sample element coupled to the ultrasonic instrument and including at least one fiber configured to collect fluorescent light emitted from the tissue, and a control system coupled to the power supply and the sample element. The control system is configured to: based on the fluorescent light, detect a type of tissue being contacted by the tip of the ultrasonic instrument; and based on the detected type of tissue, set the AC drive signal generated by the power supply to induce first pulsed ultrasonic energy in the tip.

In a further aspect, an ultrasonic surgical system includes an ultrasonic instrument having a tip and a driver coupled to the tip, the driver configured to vibrate the tip to ablate tissue from a target site responsive to receiving an AC drive signal, a sample element coupled to the ultrasonic instrument and including at least one fiber configured to collect fluorescent light emitted from the tissue, and one or more controllers. The one or more controllers are configured to: determine a first tissue characteristic of the tissue being contacted by the operative end of the tip that is indicated by the collected fluorescent light; determine a characteristic of the AC drive signal supplied to the ultrasonic instrument that corresponds to the collected fluorescent light indicative of the first tissue characteristic; determine a second tissue characteristic of the tissue being contacted by the operative end of the tip that is indicated by the characteristic of the AC drive signal; and display at least one indicator corresponding to the first and second tissue characteristics.

In a further aspect, an ultrasonic surgical system includes an ultrasonic instrument having a tip and a driver coupled to the tip, the driver configured to vibrate the tip to ablate tissue from a target site responsive to receiving an AC drive signal, a sample element coupled to the ultrasonic instrument and including at least one fiber configured to collect fluorescent light emitted from the tissue, and one or more controllers. The one or more controllers are configured to: determine a first tissue characteristic of the tissue being contacted by the operative end of the tip that is indicated by the collected fluorescent light; determine a characteristic of the AC drive signal supplied to the ultrasonic instrument that corresponds to the collected fluorescent light indicative of the first tissue characteristic; determine a second tissue characteristic of the tissue being contacted by the operative end of the tip that is indicated by the characteristic of the AC drive signal; determine whether the first tissue characteristic is inconsistent with the second tissue characteristic; and responsive to determining that the first tissue characteristic is inconsistent with the second tissue characteristic, indicate a system error.

In a further aspect, an ultrasonic surgical system includes an ultrasonic instrument having an aspiration pathway, a tip, and a driver coupled to the tip, the driver configured to vibrate the tip to ablate tissue from a target site responsive to receiving an AC drive signal, a sample element coupled to the ultrasonic instrument and including at least one fiber configured to collect fluorescent light emitted from the tissue, and one or more controllers, a sensor coupled to the aspiration pathway for measuring a characteristic of resected tissue that moves through the aspiration pathway, and a control system. The control system is configured to: determine a first tissue characteristic of tissue being contacted by the operative end of the tip that is indicated by the collected fluorescent light; determine a second tissue characteristic of resected tissue that moves through the aspiration pathway that is indicated by the sensor; determine a resection status based on the first and second tissue characteristics; and display the resection status.

### BRIEF DESCRIPTION OF THE DRAWINGS

FIG. 1 illustrates an ultrasonic surgical system incorporating an ultrasonic tool system, a tissue detection system, a navigation system, and an imaging system communicatively coupled.

FIG. 2 illustrates an ultrasonic tool system and a tissue detection system communicatively coupled.

FIG. 3 illustrates an ultrasonic instrument of an ultrasonic tool system.

FIG. 4 illustrates components that may be incorporated in a control console of an ultrasonic tool system.

FIG. 5 illustrates components that may be incorporated in an ultrasonic instrument of an ultrasonic tool system.

FIGS. 6A and 6B illustrate circuits representing current flow through an ultrasonic instrument of an ultrasonic tool system.

FIGS. 7A and 7B illustrate pulsing profiles that may be induced in an ultrasonic instrument of an ultrasonic tool system.

FIGS. 8A and 8B illustrate additional pulsing profiles that may be induced in an ultrasonic instrument of an ultrasonic tool system.

FIGS. 9A and 9B illustrate further pulsing profiles that may be induced in an ultrasonic instrument of an ultrasonic tool system.

FIG. 10 illustrates a modulation waveform that may be generated by a control console of an ultrasonic tool system to induce pulsed ultrasonic energy in an ultrasonic instrument of the ultrasonic tool system.

FIG. 11 illustrates a base AC signal that may be generated by a control console of an ultrasonic tool system to induce pulsed ultrasonic energy in an ultrasonic instrument of the ultrasonic tool system.

FIGS. 12A and 12B illustrate AC signals that may be generated by a control console of an ultrasonic tool system to induce pulsed ultrasonic energy in an ultrasonic instrument of the ultrasonic tool system.

FIG. 13 illustrates a method for inducing pulsed ultrasonic energy in an ultrasonic instrument.

FIG. 14 illustrates a method for providing tactile feedback to a practitioner maneuvering an ultrasonic instrument by inducing pulsed ultrasonic energy in the ultrasonic instrument.

FIG. 15 illustrates a graph that may be used for providing tactile feedback to a practitioner maneuvering an ultrasonic instrument by varying a pulsing frequency of pulsed ultrasonic energy induced in the ultrasonic instrument as a function of applied load.

FIG. 16 illustrates a graph representative of providing tactile feedback to a practitioner maneuvering an ultrasonic instrument by enabling and disabling pulsed ultrasonic energy in the ultrasonic instrument as a function of applied load.

FIG. 17 illustrates components that may be incorporated in a tissue detection system for detecting characteristics of tissue being contacted by an ultrasonic instrument.

FIG. 18 illustrates components that may be incorporated in a navigation system for tracking an ultrasonic instrument during a surgical procedure.

FIG. 19 illustrates a processing architecture that may be implemented by an ultrasonic surgical system.

FIG. 20 illustrates a method for operating an ultrasonic instrument based on a tracked pose of the ultrasonic instrument relative to patient anatomy.

FIG. 21 illustrates virtual boundaries and regions that may be generated to control operation of an ultrasonic instrument during a brain tumor resection procedure.

FIG. 22 illustrates virtual boundaries and regions that may be generated to control operation of an ultrasonic instrument during a spinal fusion procedure.

FIG. 23 illustrates a method for operating an ultrasonic instrument based on tissue characteristics detected by a tissue detection system coupled to the ultrasonic instrument.

FIG. 24 illustrates a method for tissue detection incorporating data from both an ultrasonic tool system and a tissue detection system.

FIGS. 25A to 25D illustrate a graphical user interface (GUI) that may be used to indicate tissue characteristics detected by an ultrasonic tool system and a tissue detection system communicatively coupled.

FIG. 26 illustrates a method for tissue detection incorporating data from each of an ultrasonic tool system, a tissue detection system, and a navigation system communicatively coupled.

FIG. 27 illustrates a GUI that may be used to indicate tissue characteristics detected by each of an ultrasonic tool system, a tissue detection system, and a navigation system communicatively coupled.

FIG. 28 illustrates a method for tracking a resection status of a surgical procedure using data from an ultrasonic tool system.

### DETAILED DESCRIPTION

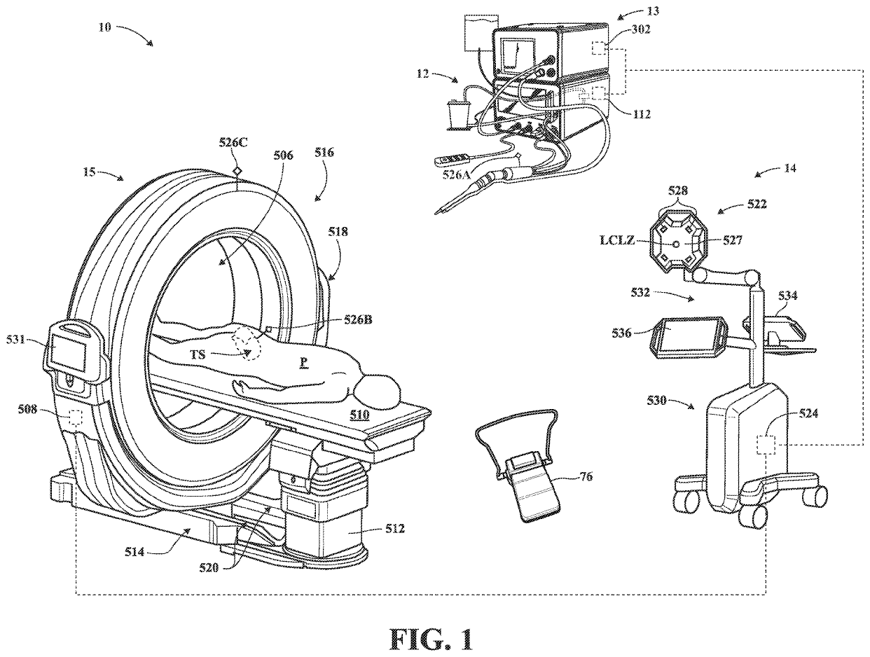

FIG. 1 illustrates an ultrasonic surgical system 10 for ablating patient tissue using ultrasonic energy during a surgical procedure, and for contemporaneously detecting characteristics of contacted tissue and/or tracking a position of one or more objects to provide surgical guidance during the surgical procedure. The surgical system 10 may include an ultrasonic tool system 12, a tissue detection system 13, a navigation system 14, and an imaging system 15. As explained in more detail below, the systems 12, 13, 14, 15 may be communicatively coupled to each other to facilitate the features of the surgical system 10 described herein. In some implementations, at least one of the tissue detection system 13, navigation system 14, or imaging system 15 may be omitted from the surgical system 10.

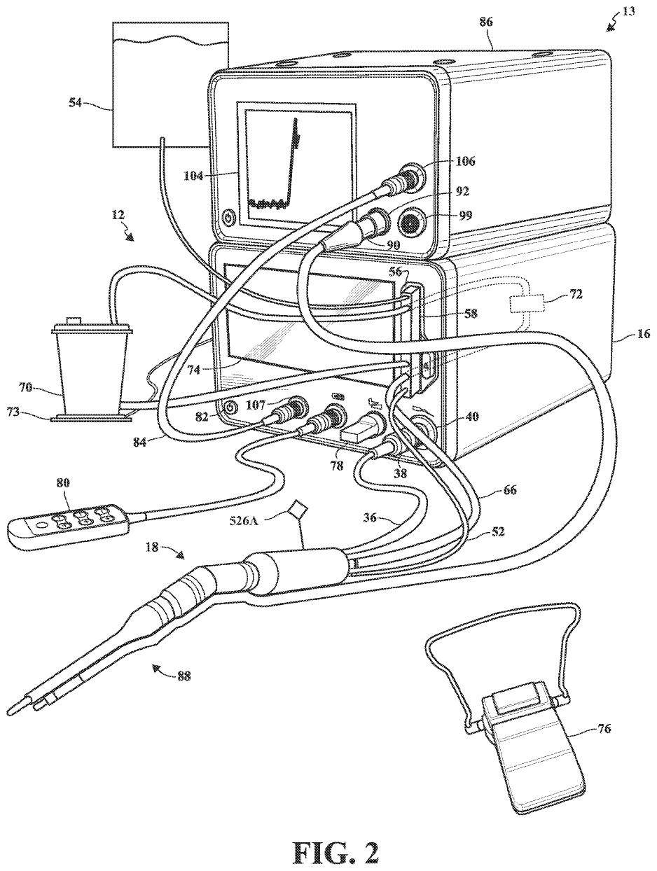

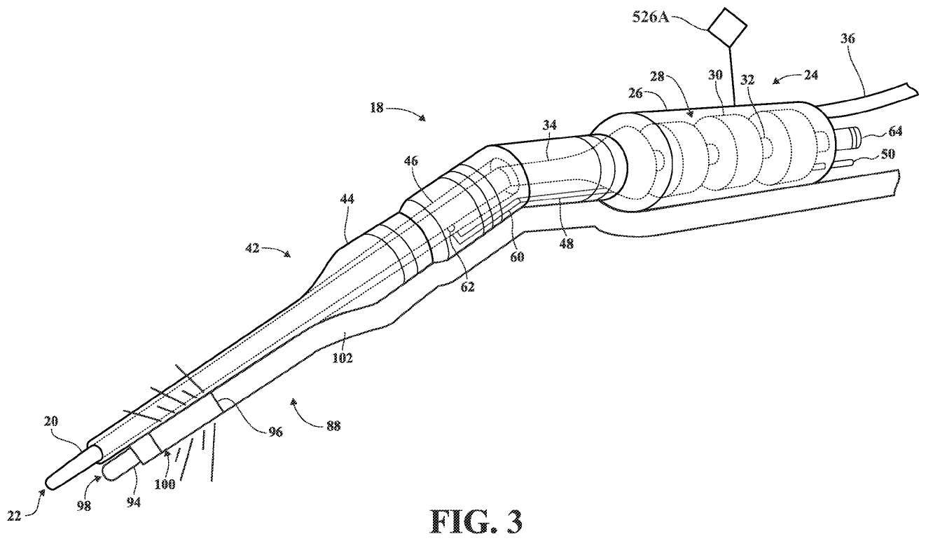

Referring to FIGS. 2 and 3, the ultrasonic tool system 12 may include an ultrasonic control console 16 and an ultrasonic instrument 18. The ultrasonic instrument 18 may include a tip 20 with a tip head 22 (also referred to as an operative end 22) configured for contacting and treating patient tissue. During operation, the ultrasonic control console 16 may generate and source an AC drive signal to the ultrasonic instrument 18 that induces ultrasonic energy in the ultrasonic instrument 18, which in turn causes the tip head 22 to rapidly vibrate. A practitioner may then position the vibrating tip head 22 against patient tissue to ablate the contacted tissue. The frequency, amplitude, and velocity of the vibrations of the tip 20 may correspond to that of the induced ultrasonic energy, which in turn may correspond to that of the AC drive signal.

The ultrasonic instrument 18 may include a handpiece 24 for being grasped by a practitioner to guide and maneuver the ultrasonic instrument 18 against patient tissue. The tip 20 may be removably coupled to the handpiece 24 so as to enable the handpiece 24 to be used with different interchangeable tips 20. Different tips 20 removably coupleable to the handpiece 24 may be configured for different types of procedures. Some tips 20 removably coupleable to the handpiece 24 may be configured for ablating soft tissue, such as by inducing cavitation in such tissue. A tip 20 configured for ablating soft tissue may define a lumen for providing suction at the surgical site through the tip 20. Some tips 20 removably coupleable to the handpiece may be configured for ablating hard tissue such as fibrous tissue and bone. A tip 20 configured for ablating hard tissue may feature a tip head 22 formed with teeth or flutes dimensioned to remove tissue via a cutting action. Tips 20 removably coupleable to the handpiece 24 may also be of different lengths for providing access to patient anatomy at different depths. Some tips 20 removably coupleable to the handpiece 24 may be designed to only vibrate longitudinally at their tip heads 22, while other tips 20 removably coupleable to the handpiece 24 may be designed to vibrate both longitudinally and torsionally and/or substantially torsionally at their tip heads 22. As described in more detail below, the surgical system 10 may be configured to consider the given tip 20 that is coupled to the handpiece 24 when detecting the characteristics of tissue being contacted by the tip 20.

The handpiece 24 may form a proximal end of the ultrasonic instrument 18, and the tip 20 coupled to the handpiece 24 may form a distal end of the ultrasonic instrument 18. “Proximal” may be understood as towards a practitioner holding the ultrasonic instrument 18 and away from the tissue to which the tip 20 is being applied, and “distal” may be understood as away from the practitioner and towards the tissue to which the tip 20 of the ultrasonic instrument 18 is being applied.

The handpiece 24 may include a housing 26 that defines a handle for the practitioner to grasp and maneuver the ultrasonic instrument 18. The housing 26 may also define a void containing a transducer 28. The transducer 28 may include one or more drivers 30, such as one or more piezoelectric crystals. The drivers 30 may be disc shaped, and may be arranged within the housing 26 end to end in a stack. Each driver 30 may be formed from a material that, upon application of an alternating electrical current, undergoes momentary expansions and contractions along the longitudinal axis of the driver 30, namely, the axis that extends between the proximally and distally directed faces of the driver 30. It is further contemplated that the drivers 30 may be realized as one or more magnetostrictive elements. Insulating discs may be disposed between and tightly abut adjacent drivers 30.

The transducer 28 may further include a tube 32, which may extend through the collinear longitudinal axes of the drivers 30 (and insulating discs, if present). To this end, each of the drivers 30 (and insulating discs) may include an internal through bore through which the tube 32 extends. A proximal end mass may be attached to the proximally directed face of the most proximally located driver 30, and may be fixedly attached to an exposed proximal end section of the tube 32. In one example, the tube 32 may be threaded at at least the proximal end section, and the proximal end mass may be a nut threaded thereon.

The handpiece 24 may also include a horn 34 at least partially disposed within the void defined by the housing 26. The horn 34 may be coupled to the distal end of the transducer 28. The horn 34 may be constructed from a rigid steel alloy, titanium or similar material. In operation, as the transducer 28 expands and contracts, the horn 34 may oscillate. The horn 34 may be removably coupled to the transducer 28. For example, the proximal end of the horn 34 may include a threaded male coupler and the distal end of the transducer 28 may include a corresponding female threaded coupler. Alternatively, the transducer 28 and the horn 34 may be permanently coupled via a weld, adhesive, or similar bonding process. Handpiece 24 may be constructed so that the stack of drivers 30 is compressed between the proximal end mass and horn 34.

The tip 20 may be removably couplable to the horn 34. For instance, the distal end of the horn 34 may include a threaded coupler configured to engage corresponding threads on the proximal end of the tip 20. It is further contemplated that other coupling methods may be utilized to removably couple the tip 20 to the horn 34. For example, the distal end of the horn 34 may comprise features that allow snap fit engagement with the tip 20.

The ultrasonic instrument 18 may be removably couplable to the ultrasonic control console 16 via an electrical cable 36. One end the electrical cable 36 may be permanently connected to the proximal end of the housing 26 of the ultrasonic instrument 18, and the other end of the electrical cable 36 may include an adapter 38 corresponding to a socket 40 of the ultrasonic control console 16. The socket 40 may be shaped to receive the adapter 38, and may include electrical contacts corresponding to electrical contacts of the adapter 38 such that when the adapter 38 is fully seated in the socket 40, an electrical connection is formed between the ultrasonic instrument 18 and the ultrasonic control console 16.

Upon actuation of the ultrasonic instrument 18, the ultrasonic control console 16 may generate and source an AC drive signal to the ultrasonic instrument 18 over the electrical cable 36. Application of the AC drive signal to the ultrasonic instrument 18 may induce ultrasonic energy in the ultrasonic instrument 18, and correspondingly may cause the tip 20 of the ultrasonic instrument 18 to vibrate.

More particularly, the ultrasonic instrument 18 may be designed so that the AC drive signal from the ultrasonic control console 16 is applied to each of the drivers 30 of the transducer 28 in parallel, which may cause the drivers 30 to simultaneously expand and contract along a longitudinal axis of the transducer 28 in accordance with the AC drive signal. The stack of drivers 30 may be between 1 and 5 cm in length. The distance, or amplitude, of movement over a single expansion/contraction cycle of the drivers 30 may be between 0.01 and 10 microns.

The horn 34 may be configured to amplify this movement. Consequently, the distal end of the horn 34 and, by extension, the tip 20 may each move back and forth along its longitudinal axis between a fully contracted position to a fully extended position, thereby producing a longitudinal vibrating motion. In some examples, the maximum peak-to-peak vibration of the tip head 22, representing a single movement from the fully contracted position to the fully extended position, may be 1000 microns, or 500 microns, or 300 microns. As previously described, some tips 20 removably coupleable to the handpiece 24 may be configured to vibrate both longitudinally and torsionally and/or substantially torsionally at their tip heads 22. Such a tip 20 may include a feature along its length, such as helical grooves, that is configured to convert the longitudinal vibrations applied to the proximal end of the tip 20 into vibrations at the tip head 22 having both a longitudinal component and a torsional component and/or having substantially only a torsional component.

To assist in reducing heat generation during an operation, the ultrasonic instrument 18 may define an irrigation pathway for supplying irrigating fluid to a distal region of the tip 20 (e.g., the tip head 22) and the surgical site. For instance, the ultrasonic instrument 18 may include an irrigation sleeve 42 adapted to be disposed around the tip 20 and removably coupled to the handpiece 24, such as the housing 26 of the handpiece 24, for supplying irrigating fluid to at least the distal region of tip 20 and the surgical site.

The irrigation sleeve 42 may include a sleeve body 44 having open proximal and distal ends and defining a lumen 46 extending between the open proximal and distal ends. The sleeve body 44 may be adapted to be coupled to the handpiece 24, such as the housing 26 of the handpiece 24, so that the tip 20 extends through the lumen 46 and out the open distal end of the sleeve body 44. For instance, the proximal end of the sleeve body 44 may be formed with a coupling feature for releasably coupling the sleeve body 44 to the distal end of the housing 26. When disposed over the tip 20 and coupled to the housing 26, the sleeve body 44 may be radially spaced from the tip 20, and may be spaced longitudinally away from the tip head 22 as described above. The components of the ultrasonic instrument 18 may be dimensioned so that during normal operation, the tip 20 does not contact the irrigation sleeve 42.

During operation of the ultrasonic instrument 18, irrigating fluid may be flowed from the handpiece 24, into the gap between the tip 20 and the sleeve body 44, and then out the open distal end of the sleeve body 44. More specifically, the handpiece 24 may include an irrigation conduit 48 running through the housing 26 from the proximal end to the distal end of the handpiece 24. The proximal end of the irrigation conduit 48 may be coupled to a fitting 50 of the ultrasonic instrument 18 that extends from a proximal end of the handpiece 24 for receiving an irrigation line 52. The irrigation line 52 may be coupled to a fluid supply 54 via a cassette 56, which may be inserted in a corresponding slot 58 of the ultrasonic control console 16. During operation of the surgical system 10, a pump of the ultrasonic control console 16 may operate on the cassette 56 to draw fluid from the fluid supply 54 into the irrigation line 52 and thereafter into the irrigation conduit 48.

The irrigation sleeve 42 may similarly include an irrigation conduit 60 in fluid communication with the lumen 46 defined by the sleeve body 44. The irrigation conduit 60 may extend from the proximal region of the sleeve body 44 and run adjacent the lumen 46 to an aperture 62 formed in a wall of the lumen 46. The aperture 62 may be positioned at an intermediary portion of the lumen 46 between the proximal and distal ends of the lumen 46, and may be configured to supply irrigating fluid from the irrigation conduit 60 into the gap between the tip 20 and the sleeve body 44. The proximal end of the irrigation conduit 60 of the irrigation sleeve 42 may be adapted to fluidly engage the distal end of the irrigation conduit 48 of the handpiece 24 when the irrigation sleeve 42 is coupled to the handpiece 24.

Accordingly, during operation of the ultrasonic instrument 18, irrigating fluid may flow from a fluid supply 54, through the irrigation line 52, fitting 50, and conduits 48, 60, and out the aperture 62 into the lumen 46. Such irrigating fluid may then run distally down the lumen 46 and out the open distal end of the sleeve body 44. In alternative examples, rather than being configured to receive irrigating fluid from the handpiece 24, the irrigation sleeve 42 may include a fitting in fluid communication with the irrigation conduit 60 and disposed on an outer surface of the sleeve body 44 for receiving the irrigation line 52 running outside of the handpiece 24. In this case, during operation of the ultrasonic instrument 18, irrigating fluid may be similarly flowed through the gap between the tip 20 and the sleeve body 44 via the fitting and out the open distal end of the sleeve body 44.

The ultrasonic instrument 18 may also define an aspiration pathway for providing suction at the distal region of the tip 20 (e.g., the tip head 22). For instance, the tube 32 of the transducer 28 may define a lumen extending from the proximal end to the distal end of the transducer 28 to create a fluid passageway through the transducer 28. The horn 34 may similarly define a lumen extending from the proximal end to the distal end of the horn 34 to create a fluid passageway through the horn 34, and the tip 20 may also define a lumen extending from the proximal end to the distal end of the tip 20 to create a fluid passageway through the tip 20. Collectively, these lumens may form at least a portion of an aspiration pathway that extends from the distal region of the tip 20 to the proximal region of the handpiece 24.

The ultrasonic instrument 18 may further include a fitting 64 coupled to the tube 32 and extending proximally from the proximal region of the handpiece 24 for receiving a suction line 66. During a procedure, suction may be applied to the fluid pathway defined by the tube 32, horn 34, and tip 20 via the fitting 64 and suction line 66 to draw the irrigating fluid applied to the surgical site and debris formed by a procedure that is entrained in the fluid towards and out of the proximal end of the handpiece 24. More specifically, the ultrasonic control console 16 may include a vacuum pump in fluid communication with a waste canister 70 via the cassette 56 when inserted in the ultrasonic control console 16, with the waste canister 70 being separately placed in fluid communication with the fluid passageway defined by the tube 32, horn 34, and tip 20, such as via a fluid passageway defined by the fitting 64, suction line 66, and cassette 56 when the cassette 56 is inserted in the ultrasonic control console 16. In this way, the vacuum pump may apply a suction to the fluid passageway defined by the tube 32, horn 34, and tip 20 via the waste canister 70, cassette 56, suction line 66, and fitting 64, thereby drawing materials from the surgical site through the aforementioned fluid passageways into the waste canister 70. The suction may also function to draw tissue towards the tip head 22, which may enhance the effectiveness of the tip 20 in treating patient tissue.

The ultrasonic tool system 12 may further include one or more sensors associated with the aspiration pathway for measuring one or more characteristics of resected tissue that moves through the pathway. As described in more detail below, the surgical system 10 may be configured to use the information generated by these sensor(s) to track a resection status of a surgical procedure.

For example, a portion of the aspiration passageway between the waste canister 70 and the ultrasonic instrument 18 may pass through a suction sensor 72 of the ultrasonic control console 16. The suction sensor 72 may be configured to generate data indicative of a presence and volume of patient tissue that passes through the aspiration pathway. In some instances, the suction sensor 72 may include a flow sensor. Additionally or alternatively, the suction sensor 72 may include a scanner for scanning the tissue that passes through the aspiration pathway and generating corresponding data indicative of characteristics of the tissue, such as the size, volume, and/or type of tissue. For instance, the scanner may include an IR transceiver and/or a fluorescence emitter/collector, such as similar to that described below, each of which may be configured to excite the resected tissue passing through the aspiration pathway with light and then collect light signals emitted by the tissue as a result of the excitation to determine one or more of the above tissue characteristics. In some instances, targeted tissue may be dyed to have distinct optical properties prior to a surgical procedure so as to enable the suction sensor 72 to differentiate resected tissue corresponding to the targeted tissue from resected tissue corresponding to non-targeted tissue. Additionally or alternatively, the sensor(s) associated with the aspiration pathway may include a weight sensor 73 configured to generate data indicative of a weight of patient tissue that has been resected through the aspiration pathway and deposited into the waste canister 70.

The ultrasonic control console 16 may also include a display 74 for presenting information to the practitioner. Non-limiting examples of presented information may include an identification of the ultrasonic instrument 18, or more particularly of the handpiece 24 and/or tip 20, currently connected to the ultrasonic control console 16, and an operating state of the ultrasonic tool system 12 and/or surgical system 10. The display 74 may be a touch screen display that enables the practitioner to provide input to the ultrasonic control console 16, such as via on-screen control elements. A practitioner may interact with the on-screen control elements to set operational parameters of the ultrasonic tool system 12, such as a maximum ultrasonic energy level, a suction level, and an irrigation level for the ultrasonic instrument 18.

The ultrasonic tool system 12 may also include one or more actuation devices coupled to the ultrasonic control console 16. Upon activation by the practitioner, each of the actuation devices may cause the ultrasonic control console 16 to generate and source the AC drive signal to the ultrasonic instrument 18 that induces ultrasonic energy in the ultrasonic instrument 18, and correspondingly causes the tip 20 of the ultrasonic instrument 18 to vibrate according to the set operational parameters.

For instance, the one or more actuation devices may include a foot pedal 76. The foot pedal 76 may be wirelessly connected to the ultrasonic control console 16, such as via an adapter 78 connected to the ultrasonic control console 16. Upon being depressed, the foot pedal 76 may transition from an off position to an active position, and may correspondingly communicate an actuation signal to the ultrasonic control console 16 that indicates the depression. In some instances, the communicated actuation signal may vary with the extent to which the foot pedal 76 is depressed, such as to enable the practitioner to vary the ultrasonic energy level induced in the ultrasonic instrument 18 up to the set maximum ultrasonic energy level via the foot pedal 76. Responsive to receiving the actuation signal, the ultrasonic control console 16 may generate and source an AC drive signal to the ultrasonic instrument 18 that causes the tip 20 to vibrate according to the current settings of the ultrasonic control console 16 and/or the extent of the depression indicated by the actuation signal.

The ultrasonic tool system 12 may also include a remote control 80 coupled to the ultrasonic control console 16. Similar to the touch screen display 74, the remote control 80 may include practitioner-selectable elements for providing input to the ultrasonic control console 16. For instance, the remote control 80 may include buttons for setting the operational parameters of the ultrasonic tool system 12, such as the maximum ultrasonic energy level, suction level, and irrigation level for the ultrasonic instrument 18. The remote control 80 may also include a power button for turning on and off the ultrasonic control console 16. Additionally, or alternatively, the ultrasonic control console 16 may include an integrated power button 82 for turning on and off the ultrasonic control console 16.

Still referring to FIGS. 2 and 3, the tissue detection system 13 may be communicatively coupled to the ultrasonic tool system 12, such as via an electrical cable 84 connecting the ultrasonic control console 16 of the ultrasonic tool system 12 with a tissue detection control console 86 of the tissue detection system 13. The tissue detection system 13 may be configured to detect a type of tissue being contacted by the tip 20 of the ultrasonic instrument 18, and may be configured to cooperate with the ultrasonic tool system 12 to control operation of the surgical system 10, or more particularly the ultrasonic instrument 18, based on the detected type of tissue.

The tissue detection system 13 may include the tissue detection control console 86 and a sample element 88. The sample element 88 may be connected to the tissue detection control console 86, such as via a connector 90 integrated with the sample element 88 and inserted into a corresponding socket 92 of the tissue detection control console 86. The sample element 88 may be coupled to the ultrasonic instrument 18, such as along the length of the handpiece 24 and/or the irrigation sleeve 42 as shown in the illustrated example. For example and without limitation, the sample element 88 may be coupled to the ultrasonic instrument 18 using an adhesive, such as in the form of a sticker or glue, or via one or more fixation elements wrapped around the ultrasonic instrument 18 and the sample element 88, such fixation elements being periodically spaced along the length of the ultrasonic instrument 18.

The sample element 88 may include an excitation fiber 94, and may include a detection indicator 96. During a surgical procedure, the tissue detection control console 86 may be configured to illuminate tissue adjacent to or being contacted by the operative end 22 of the tip 20 with excitation light via the excitation fiber 94. To this end, the excitation fiber 94 may run the length of the sample element 88 such that when the sample element 88 is coupled to the ultrasonic instrument 18, a distal region 98 of the excitation fiber 94 is adjacent the operative end 22 of the tip 20 so as to allow excitation light to be delivered to the tissue adjacent to or being contacted by the operative end 22 of the tip 20. As shown in the illustrated example, the sample element 88 may be coupled to the ultrasonic instrument 18 in a manner such that there is no direct contact between the tip 20 and the distal portion of the sample element 88. For example, the sample element 88 may terminate adjacent a portion of the irrigation sleeve 42 proximal the operative end 22 of the tip 20. In another example, the sample element 88 may extend past the irrigation sleeve 42, but be arranged such that there is adequate empty space between the operative end 22 of the tip 20 and the sample element 88 to prevent contact therebetween.

Responsive to being illuminated with excitation light at a given wavelength, different tissues may exhibit different levels and/or different wavelengths of fluorescence. For instance, prior to a surgical procedure involving the removal of tumorous tissue, Aminolevulinic Acid (5-ALA) may be given to the patient a couple hours before surgery. 5-ALA is a compound that occurs naturally in the hemoglobin synthesis pathway. In cancer cells, the hemoglobin synthesis is disrupted and the pathway stalls at an intermediate compound called Protoporphyrin IX (PPIX). When illuminated with excitation light at a certain wavelength (e.g., blue light) from the excitation fiber 94, tumor cells containing PPIX may absorb the excitation light and emit fluorescence having specific optical characteristics (e.g., red fluorescence of a minimum intensity level), thereby indicating the presence of tumorous cells. As a further example, Idocyanine Green (ICG) may be administered to a patient prior to a surgery, and may bond to plasma protein found in blood. When illuminated with excitation light at a certain wavelength (e.g., near infrared light), the ICG may emit fluorescence having specific optimal characteristics (e.g., near infrared fluorescence of a minimum intensity level), thereby indicating the presence of a blood vessel. Other fluorophores that may be excited to detect various types of tissue include Hypericin and Hexvix.

The tissue detection control console 86 may thus be configured to illuminate the tissue adjacent to or being contacted by the operative end 22 of the tip 20 with excitation light at one or more wavelengths via the excitation fiber 94, and thereafter collect fluorescent light emitted from the illuminated tissue via the excitation fiber 94. In alternative examples, the tissue detection system 13 may include a separate fiber for collecting the emitted fluorescent light, which may be incorporated into a separate collection element running alongside the sample element 88 and connected to a socket 99 of the tissue detection control console 86. In either configuration, the tissue detection control console 86, such as via an integrated spectrometer, may be configured to convert the collected light into electrical signals interpreted by the tissue detection control console 86. The electrical signals may indicate the intensity of various fluorophores contained in the collected light, and the tissue detection control console 86 may be configured to analyze the electrical signals to determine at least one characteristic of the tissue adjacent to or in contact with the operative end 22 of the tip 20 of the ultrasonic instrument 18 that is indicated by the electrical signals, such as the type of tissue. For instance, the tissue detection control console 86 may be configured to compare the intensity of red fluorescence indicated by the electrical signals to a minimum intensity threshold associated with tumorous tissue. Responsive to the comparison indicating that the intensity of the red fluorescence is greater than or equal to the minimum intensity threshold, the tissue detection control console 86 may be configured to determine that the operative end 22 of the tip 20 of the ultrasonic instrument 18 is currently contacting tumorous tissue. If not, then the tissue detection control console 86 may be configured to determine that the operative end 22 of the tip 20 of the ultrasonic instrument 18 is currently contacting non-tumorous tissue.

Responsive to determining the presence of a tissue characteristic indicative of a given type of tissue, the tissue detection control console 86 may be configured to generate an activation signal that causes the detection indicator 96 to emit light, thereby providing a real-time indication to the healthcare professional of the presence of the given type of tissue. As shown in the illustrated example, the detection indicator 96 may be illuminated by an indicator fiber 100 that, like the excitation fiber 94, runs the length of the sample element 88. The detection indicator 96 may be situated proximal to the distal portion of the sample element 88 to ensure that the practitioner is able to view the detection indicator 96 as the practitioner is resecting tissue. The detection indicator 96 may be transparent, and may correspond to a removed portion of a jacket of the sample element 88. In one example, the sample element 88 may include a co-axial fiber with a central core and an outer channel covered by the jacket. The excitation fiber 94 may be disposed within the central core while the indicator fiber 100 may be disposed within the outer channel. A portion of the jacket 102 of the sample element 88 may be removed such that the indicator fiber 100 may illuminate light through the sidewalls of the outer channel to light up the detection indicator 96.

The indicator fiber 100 may be coupled to receive light from an excitation source integral with the tissue detection control console 86, with the light being at a different wavelength than the excitation light used to illuminate the tissue. Responsive to detection of a tissue characteristic indicative of a given type of tissue, the tissue detection control console 86 may be configured to transmit light down the indicator fiber 100 to illuminate the detection indicator 96 via the excitation source of the tissue detection control console 86. In some examples, the detection indicator 96 may be illuminated with varying colors of light depending on the detected tissue characteristic. For example, the tissue detection control console 86 may be configured to control the excitation source (e.g., one or more LEDs) to emit green light (e.g., wavelengths of about 520-564 nm) from the detection indicator 96 when the PPIX fluorophore above a threshold is detected, and a yellow light (e.g., wavelengths of about 565-590 nm) from the detection indicator 96 when the ICG fluorophore above a threshold is detected.

The tissue detection control console 86 may similarly include a display 104 for presenting information to the practitioner. One non-limiting example of presented information may include an identification of the tissue characteristic detected by the tissue detection system 13. The display 104 may be a touch screen display that enables the practitioner to provide input to the tissue detection control console 86, such as via on-screen control elements. A practitioner may interact with the on-screen control elements to set operational parameters of the tissue detection system 13, such as a tissue type targeted for ablation (e.g., tumorous tissue) and/or characteristics of a tissue type targeted for ablation (e.g., minimum and type of fluorophore(s) corresponding to the tissue). In this way, the tissue detection control console 86 may illuminate the detection indicator 96 in a particular manner responsive to determining that the operative end 22 of the tip 20 is contacting or adjacent to tissue corresponding to the set tissue type and/or characteristics.

As described above, the tissue detection system 13 may be in communication with the ultrasonic tool system 12. For example, such communication may be established through an electrical port 106 integral with the tissue detection control console 86, from which the electrical cable 84 may extend to a corresponding electrical port 107 of the ultrasonic control console 16. Alternatively, the communication link between the control consoles 16, 86 may be established wirelessly.

The tissue detection system 13 and ultrasonic tool system 12 may be configured to cooperate to regulate operation of the surgical system 10, or more particularly the ultrasonic instrument 18. For example, like the tissue detection control console 86, the ultrasonic control console 16 may be configured to monitor one or more characteristics of the tissue being contacted by the operative end 22 of the tip 20. More specifically, characteristics of the AC drive signal sourced to the ultrasonic instrument 18 to vibrate the tip 20 may indicate a characteristic of the tissue being contacted by the operative end 22 of the tip 20, such as a mechanical impedance (e.g., stiffness) of the contacted tissue. The ultrasonic control console 16 may thus be configured to monitor one or more characteristics of the AC drive signal, and determine at least one characteristic of the tissue being contacted by the operative end 22 of the tip 20 based on the one or more monitored characteristics. In addition to displaying at least one indicator corresponding to the tissue characteristics determined by the ultrasonic tool system 12 and the tissue detection system 13, such as on one or more of the displays 74, 104, the tissue detection system 13 and/or ultrasonic tool system 12 may be configured to determine whether the tissue characteristics determined by the systems 12, 13 are inconsistent. If so, then the tissue detection system 13 and/or ultrasonic tool system 12 may be configured to trigger a system error, which may include preventing operation of the ultrasonic instrument 18 until the surgical system 10 is restarted via user input. These and other functionalities of the surgical system 10 are described in more detail below.

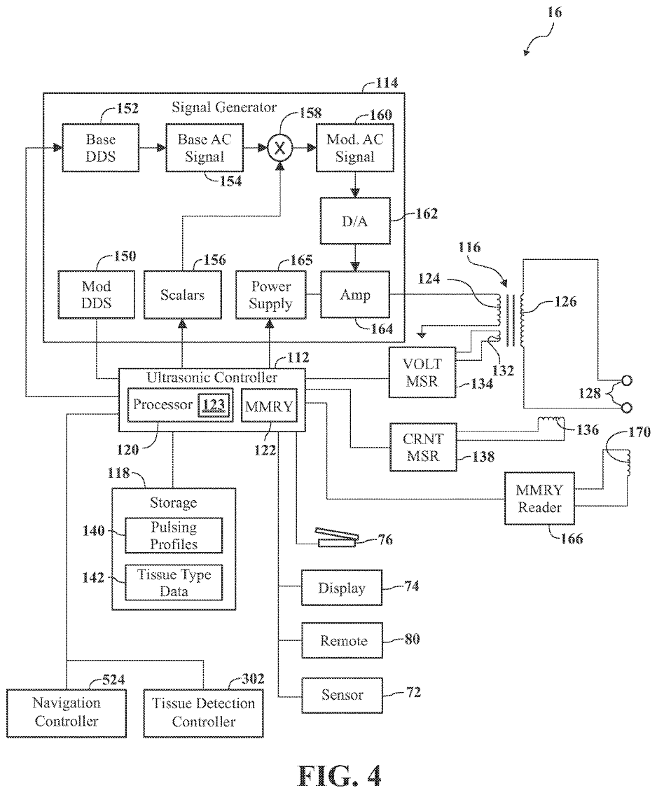

FIG. 4 illustrates components that may be integral with the ultrasonic control console 16. The control console 16 may include an ultrasonic controller 112, a signal generator 114, a transformer 116, and console storage 118. In general, the signal generator 114 and transformer 116 may form a power supply of the control console 16 that is configured to generate the AC drive signal supplied to the drivers 30 of the ultrasonic instrument 18, with the output of these components being regulated by the ultrasonic controller 112.

The ultrasonic controller 112 may include a processor 120 and memory 122. The processor 120 may include one or more devices selected from microprocessors, micro-controllers, digital signal processors, microcomputers, central processing units, field programmable gate arrays, programmable logic devices, state machines, logic circuits, analog circuits, digital circuits, and/or any other devices that manipulate signals (analog or digital) based on operational instructions read into memory 122 and executed, such as from the console storage 118. The memory 122 may include a single memory device or a plurality of memory devices including, but not limited to, read-only memory (ROM), random access memory (RAM), volatile memory, non-volatile memory, static random-access memory (SRAM), dynamic random-access memory (DRAM), flash memory, cache memory, and/or any other device capable of storing information. The console storage 118 may include one or more persistent data storage devices such as a hard drive, optical drive, tape drive, non-volatile solid-state device, and/or any other device capable of persistently storing information. Although shown separate from the ultrasonic controller 112 in the illustrated example, in addition or alternatively, the console storage 118 may be included in the ultrasonic controller 112, such as in communication with the processor 120.

The ultrasonic controller 112 may be configured to implement the functions, features, processes, and methods of the control console 16 described herein. More specifically, the processor 120 of the ultrasonic controller 112 may operate under control of software programs 123 embodied by computer-executable instructions, which may reside in the console storage 118 and be read into the memory 122 for execution by the processor 120. The computer-executable instructions may be compiled or interpreted from a variety of programming languages and/or technologies, including, without limitation, and either alone or in combination, Java, C, C++, C#, Objective C, Fortran, Pascal, Java Script, Python, Perl, and PL/SQL. The computer-executable instructions may be configured, upon execution by the processor 120, to cause the processor 120 to implement the functions, features, processes, and methods of the control console 16 described herein. In this way, the ultrasonic controller 112, or more particularly the processor 120, may be configured to implement the functions, features, processes, and methods of the control console 16 described herein.

For instance, the ultrasonic controller 112, such as upon execution of the software programs 123 by the processor 120, may be configured to control the level of ultrasonic energy induced in the ultrasonic instrument 18, and correspondingly control the vibrations of the tip 20, by regulating the AC drive signal sourced to the ultrasonic instrument 18 from the control console 16. More particularly, during operation of the ultrasonic tool system 12, the ultrasonic controller 112 may be configured to output one or more control signals to the signal generator 114 that correspond to a target AC drive signal to be sourced from the control console 16 to the ultrasonic instrument 18. The signal generator 114 may be configured to responsively generate an AC signal, such as using direct digital synthesis (DDS) and one or more amplifiers, across a primary winding 124 of the transformer 116. The AC signal may be proportional to the target AC drive signal indicated by the one or more control signals, and may thus induce the target AC drive signal across a secondary winding 126 of the transformer 116, which may be coupled to the ultrasonic instrument 18, such as through electrical contacts 128.

Referring to FIG. 5, the electrical contacts 128 may be integral with the socket 40 of the control console 16. Corresponding electrical contacts 130 may be integral with the adapter 38 of the electrical cable 36. The electrical contacts 130 may also be electrically connected to opposing ends of each driver 30 of the ultrasonic instrument 18. When the adapter 38 is fully seated in the socket 40, the electrical contacts 128, 130 may become aligned and form an electrical connection, and may thereby apply the AC drive signal developed across the secondary winding 126 of the transformer 116 to each driver 30 to induce ultrasonic energy in the ultrasonic instrument 18, and correspondingly cause vibrations of the tip 20.

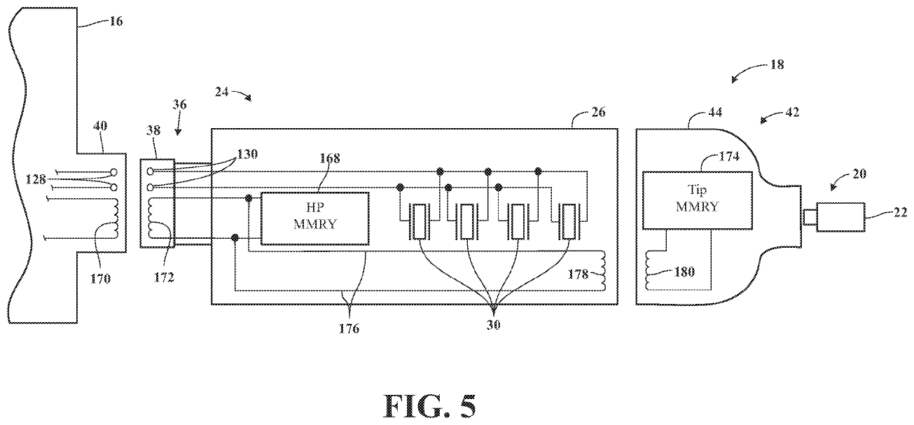

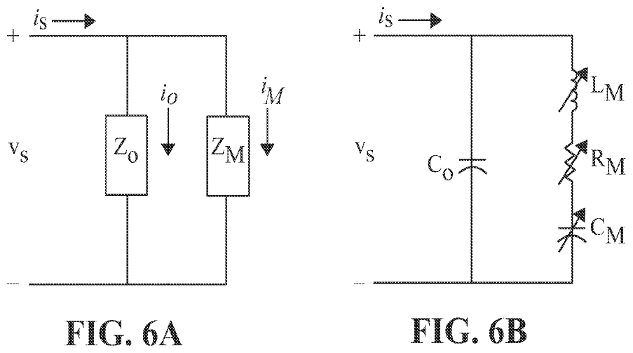

FIGS. 6A and 6B show circuits illustrating the flow of current through the ultrasonic instrument 18 when an AC drive signal is sourced to the ultrasonic instrument 18 from the control console 16. As shown in the illustrated examples, the current i_{S} of the sourced AC drive signal may be broken down into two components: a current i_{O} applied to the drivers 30 of the ultrasonic instrument 18 and an equivalent of current i_{M} applied to the mechanical components of the ultrasonic instrument 18 (also referred to herein as “mechanical current i_{M}”). The mechanical components of the ultrasonic instrument 18 may include those components that vibrate in response to the sourced AC drive signal to treat patient tissue, such as and without limitation, the drivers 30, tube 32, horn 34, tip 20, and proximal end mass described above.

The impedance Z_{O} provided by the drivers 30 may be primarily capacitive. Accordingly, the drivers 30 may be represented by a capacitor with capacitance C_{O}. The capacitance C_{O} of the drivers 30 may remain substantially constant during operation of the ultrasonic instrument 18, and may thus be determined and provided to the control console 16 in advance of an operation, such as upon connection of the ultrasonic instrument 18 to the control console 16, so as to tailor operation of the control console 16 to the specific handpiece 24 of the ultrasonic instrument 18. Additionally or alternatively, the control console 16 may be configured to periodically measure the capacitance C_{O} of the driver 30 during operation of the ultrasonic instrument 18 to enable even further precision.

The equivalent of impedance Z_{M} provided by the mechanical components of the ultrasonic instrument 18 (also referred to herein as “mechanical impedance Z_{M}”) may include an inductive component, a resistive component, and a capacitive component. Accordingly, the mechanical components may be represented by an inductor with inductance L_{M}, a resistor with resistance R_{M}, and a capacitor with capacitance C_{M}. The inductance L_{M}, resistance R_{M}, and capacitance C_{M} may vary with operation of the ultrasonic instrument 18, and at least the resistance R_{M} (also referred to herein as “mechanical resistance R_{M}”) may vary as a function of the load applied to the tip 20, such as by contacted patient tissue and/or irrigating fluid provided via the irrigation sleeve 42. In other words, the mechanical impedance Z_{M}, or more particularly mechanical resistance R_{M}, may vary based on the firmness of the tissue to which the tip 20 is applied, and/or based on the force in which the practitioner applies the ultrasonic instrument 18 to the tissue, and/or based on the flow rate of irrigating fluid running through the sleeve 42.

The ultrasonic energy induced in the ultrasonic instrument 18, and correspondingly the vibrations of the tip 20, may be proportional to the mechanical current i_{M}. For instance, the frequency of the vibrations at the tip head 22 may be equal to the frequency of the mechanical current i_{M}, and when the ultrasonic instrument 18 is operating at resonance, the peak-to-peak displacement of the tip head 22 in microns may be approximately 180% to 220% of the amplitude of the mechanical current i_{M} in milliamps, depending on the gain of the tip 20. As an example, a mechanical current i_{M} at the resonant frequency of the ultrasonic instrument 18 and with an amplitude of 150 milliamps may induce the tip head 22 of a given tip 20 to vibrate back and forth along a path of travel that is approximately 330 microns.

The ultrasonic controller 112 may thus cause vibrations in the tip 20 with a target frequency and displacement level by generating a control signal to the signal generator 114 that causes the signal generator 114 source an AC drive signal to the ultrasonic instrument 18 that induces a mechanical current i_{M} in the ultrasonic instrument 18 with the target frequency and an amplitude corresponding to the target displacement level. To this end, the ultrasonic controller 112 may be configured to implement two control loops to induce target vibrations in the tip 20, namely, a control loop for regulating the frequency of the mechanical current i_{M} induced in the ultrasonic instrument 18 by the AC drive signal, and a control loop for regulating the level or amplitude of the mechanical current i_{M} induced in the ultrasonic instrument 18 by the AC drive signal. Each control loop may incorporate a PID controller for efficiently adjusting the AC drive signal to achieve the desired values, and may have an iterative loop time of approximately 400 microseconds.

Using Ohm's law, the ultrasonic controller 112 may be configured to calculate the level of mechanical current i_{M} induced in the ultrasonic instrument 18 using the following Equation:

$${i}_{M} = {i}_{S} - j 2 π {fC}_{o} {v}_{s} \qquad ( 1 )$$

where is i_{S} the current of the AC drive signal sourced to the ultrasonic instrument 18, f is the frequency of the AC drive signal, Cₒ is the capacitance of the drivers 30, and vₛ is the voltage of the AC drive signal. An explanation for Equation (1) is provided in Applicant's U.S. Pat. No. 10,016,209, the contents of which are hereby incorporated by reference herein in their entirety. Assuming the frequency f of the AC drive signal has been previously set to achieve a desired vibratory characteristic of the ultrasonic instrument 18 (e.g., resonance), the ultrasonic controller 112 may induce a target level of mechanical current i_{M}, and correspondingly target vibrations of the tip 20, by setting the voltage vₛ of the AC drive signal so that Equation (1) results in the target level of mechanical current i_{M}.

As mentioned above, a characteristic integral with the ultrasonic instrument 18 is the mechanical resonant frequency of the ultrasonic instrument 18. The mechanical resonant frequency is a frequency at which the distal end of the tip 20 undergoes vibratory motions of a peak range. In other words, assuming other electrical characteristics remain constant, at the resonant frequency, the tip 20 undergoes a motion that is larger in magnitude than a motion that would occur if the drivers 30 were vibrated at a frequency less than or greater than the resonant frequency. For a tip 20 that vibrates longitudinally, the peak range may be understood as the largest back and forth distance of the distal end of the tip 20.

The Applicant's U.S. Pat. No. 10,016,209 also discloses a means for tracking the resonant frequency of the ultrasonic instrument 18, which may vary during operation of the ultrasonic instrument 18. In particular, the ultrasonic instrument 18 may be considered as operating at resonance when the real part of the ratio of the current i_{O} through the drivers 30 to the mechanical current i_{M} is substantially equal to zero. The ultrasonic controller 112 may thus be configured to determine the resonant frequency of the ultrasonic instrument 18 by determining a value for the frequency f of the AC drive signal such that the following Equation is true:

$$R e \{ \frac{j 2 π {fC}_{o}}{{i}_{s} - j 2 π {fC}_{o}} \} \approx 0 \qquad ( 2 )$$

where is i_{S} the current of the AC drive signal sourced to the ultrasonic instrument 18 and Cₒ is the capacitance of the drivers 30. Responsive to determining the resonant frequency of the ultrasonic instrument 18, such as using Equation (2), the ultrasonic controller 112 may be configured to set the frequency of the AC drive signal to the determined resonant frequency, thereby causing the ultrasonic instrument 18 to operate at resonance.

The ultrasonic controller 112 may also be configured to track and set the frequency of the AC drive signal according to other vibratory characteristics inherent in the ultrasonic instrument 18, such as the anti-resonant frequency of the ultrasonic instrument 18. In this case, the ultrasonic controller 112 may be configured to determine a value for the frequency f such that the left side of Equation (2) substantially equals one.

As the frequency of the AC drive signal is adjusted to follow a target vibratory characteristic of the ultrasonic instrument 18 such as resonance, the level of the mechanical current i_{M} induced in the ultrasonic instrument 18 may vary. Accordingly, to induce target ultrasonic energy in the ultrasonic instrument 18, the ultrasonic controller 112 may be configured to repeatably alternate between or perform in parallel the operations of regulating the frequency of the AC drive signal based on Equation (2) and setting the voltage vₛ of the AC drive signal so that the mechanical current i_{M}, calculated according to Equation (1), corresponds to the target ultrasonic energy.

To this end, and referring again to FIG. 4, the ultrasonic controller 112 may be configured to receive feedback data corresponding to the AC drive signal being sourced to the ultrasonic instrument 18, such as via one or more sensors integral with the control console 16. The ultrasonic controller 112 may then be configured to induce target ultrasonic energy in the ultrasonic instrument 18, and correspondingly target vibrations of the tip 20, based on the received data, such as by feeding the received data into the loops that regulate the frequency and voltage vₛ of the AC drive signal using Equations (1) and (2) respectively.

More particularly, the control console 16 may include a sensor for measuring the voltage vₛ of the AC drive signal sourced to the ultrasonic instrument 18, which may include a tickler coil 132 adjacent to or integral with the transformer 116. The tickler coil 132 may be connected to a voltage measuring circuit 134 of the control console 16, which in turn may be connected to the ultrasonic controller 112. The signal across tickler coil 132 may have a known relationship to the voltage vₛ of the AC drive signal being sourced to the ultrasonic instrument 18. Based on the signal across the tickler coil 132, the voltage measuring circuit 134 may generate and communicate a signal to the ultrasonic controller 112 representative of the magnitude and phase of the voltage vₛ of the AC drive signal being sourced to the ultrasonic instrument 18. The ultrasonic controller 112 may thus be configured to measure the voltage vₛ of the AC drive signal via the voltage measuring circuit 134 and tickler coil 132, and to generate control signals for regulating the AC drive signal based thereon.

The control console 16 may also include a sensor for measuring the current iₛ of the AC drive signal being sourced to the ultrasonic instrument 18, which may include a coil 136 located in close proximity to one of the conductors that extends from the secondary winding 126 of the transformer 116 to the ultrasonic instrument 18. The coil 136 may be connected to a current measuring circuit 138 of the control console 16, which in turn may be connected to the ultrasonic controller 112. The signal across the coil 136 may have a known relationship to the current iₛ of the AC drive signal being sourced the ultrasonic instrument 18. Based on the signal across coil 136, the current measuring circuit 138 may produce and communicate to the ultrasonic controller 112 a signal representative of the magnitude and phase of the current iₛ of the AC drive signal being sourced to the ultrasonic instrument 18. The ultrasonic controller 112 may thus be configured to measure the current iₛ of the AC drive signal via the current measuring circuit 138 and coil 136, and to generate control signals for regulating the AC drive signal based thereon.

In addition to software programs 123 embodied by computer-executable instructions, the console storage 118 may store data supporting the functions, features, processes, and methods of the control console 16 described herein. For instance, the console storage 118 may include data defining one or more pulsing profiles 140 for inducing pulsed ultrasonic energy in the ultrasonic instrument 18 as described in more detail below, and may include tissue type data 142. The tissue type data 142, also described in more detail below, may associate varying types of tissue with varying characteristics of the ultrasonic instrument 18, or more particularly of varying characteristics of the AC drive signal supplied to the ultrasonic instrument 18, that are indicative that the operative end 22 of the ultrasonic instrument 18 is contacting the type of tissue.

To ablate tissue effectively, the ultrasonic control console 12, or more particularly the ultrasonic controller 112, may cause the tip 20 of the ultrasonic instrument 18 to vibrate at a relatively high velocity. For instance, at full power, the ultrasonic control console 12 may cause vibrations of the tip 20 with a frequency between 20 and 40 kHz and peak-to-peak displacement of about 300 microns. While vibrating the tip 20 at this velocity may enable the ultrasonic instrument 18 to emulsify hard tissues such as fibrous tissue and bone, maintaining this velocity over the large number of vibratory cycles that the tip 20 undergoes during an operation may also generate a large amount of heat in the ultrasonic instrument 18 and at the surgical site. Such heat may affect operation of the ultrasonic instrument 18 and increase trauma to surrounding tissues desired to remain intact.

However, when the tip 20 is vibrated at a constant velocity to resect hard tissue, each vibratory cycle of the tip 20 may not cause an equivalent amount of resection. Rather, a large number of the vibratory cycles may merely add to the heat generation at the surgical site and not actually resect any tissue. It may thus be possible to reduce heat generation while maintaining an effective resection rate of hard tissue by periodically reducing the ultrasonic energy induced in the ultrasonic instrument 18, such as according to one of the predefined pulsing profiles 140 stored by the ultrasonic console storage 118. In some examples, the pulsing profiles 140 may be similar to that described in Applicant's PCT Publication No. WO 2022/072903 A1, the contents of which are hereby incorporated by reference herein in their entirety.

Each pulsing profile 140 may define a pattern of ultrasonic energy to be induced in the ultrasonic instrument 18, with the ultrasonic energy pattern including several ultrasonic energy pulses peaking at a maximum ultrasonic energy level set for the ultrasonic instrument 18 and interspaced by periods of ultrasonic energy at a minimum ultrasonic energy level set for the ultrasonic instrument 18. In some examples, the maximum ultrasonic energy level may be set by the practitioner, and the minimum ultrasonic energy level may be defined by the pulsing profile relative to the maximum ultrasonic energy level.

The ultrasonic energy induced in the ultrasonic instrument 18 may cause the tip 20 to vibrate with a frequency, amplitude, and velocity corresponding to that of the induced ultrasonic energy, which in turn may correspond to that of the AC drive signal. For a given pulsing profile 140, a peak vibration amplitude and velocity may occur in the tip 20 when the maximum ultrasonic energy level is induced in the ultrasonic instrument 18, which may be set to a level sufficient for resecting the type of target tissue. The periodic reductions of ultrasonic energy induced in the ultrasonic instrument 18 according to the pulsing profile 140 may cause periodic reductions of the vibration amplitude and velocity of the tip 20 from the peak amplitude and velocity, which may reduce heat generation in the ultrasonic instrument 18 and at the surgical site while maintaining an acceptable resection rate. In other words, implementation of a given pulsing profile 140 may reduce the number of vibratory cycles that the tip 20 moves at a peak velocity relative to inducing ultrasonic energy in the ultrasonic instrument 18 that is maintained at the set maximum ultrasonic energy level, leading to a reduction in frictional heat generation.

In addition to reducing heat generation when cutting hard tissue, periodically reducing ultrasonic energy induced in the ultrasonic instrument 18 according to a predetermined pulsing profile 140 may enable finer resection control when applying the tip 20 to certain tissues, such as soft tissues, by causing vibrations of the tip 20 that slow resection rates of firmer tissues while substantially maintaining resection rates of softer tissues. In other words, the predefined pulsing profiles 140 may provide improved tissue selectivity.

Different pulsing profiles 140 may be designed for different situations, such as targeting certain types of tissue for ablation and/or providing increased tactile feedback to the practitioner when cutting hard tissue such as bone. In some examples, a practitioner may select a desired pulsing profile 140 for operating the ultrasonic instrument 18, such as by interacting with the display 74 of the control console 16. Responsive to receiving selection of a given pulsing profile 140, the ultrasonic controller 112 may be configured to retrieve the pulsing profile 140 from the console storage 118, and then generate and source an AC drive signal to the ultrasonic instrument 18 that induces ultrasonic energy in the tip 20 according to the retrieved pulsing profile 140.

Each pulsing profile 140 stored in the console storage 118 may be configured to induce ultrasonic energy in the ultrasonic instrument 18 that includes a series of ultrasonic energy pulses. More particularly, each pulsing profile 140 may indicate varying target levels for the ultrasonic energy induced in the ultrasonic instrument 18 as a function of time so as to form a series of ultrasonic energy pulses peaking at a maximum ultrasonic energy level determined for the ultrasonic instrument 18 and interspaced by ultrasonic energy at a minimum ultrasonic energy level determined for the ultrasonic instrument 18. For instance, each pulsing profile 140 may indicate varying target levels for an upper envelope of the induced ultrasonic energy as a function of time, or may indicate target RMS values for the induced ultrasonic energy as a function of time. To implement a given pulsing profile 140, the ultrasonic controller 112 may thus be configured to generate and source an AC drive signal to the ultrasonic instrument 18 that induces ultrasonic energy in the ultrasonic instrument 18 according to the varying target levels indicated by the given pulsing profile 140.

Each pulsing profile 140 stored in the console storage 118 may include one or more pulsing parameter settings specific to the pulsing profile 140. The pulsing parameter(s) may be used by the ultrasonic controller 112 for regulating the ultrasonic energy pulses induced in the ultrasonic instrument 18, and may include, for example and without limitation, one or more of a factor for determining a minimum ultrasonic energy level for the induced pulsed ultrasonic energy, a pulse shape, a duty cycle, and a pulsing frequency.

The minimum energy factor of each pulsing profile 140 may define a minimum ultrasonic energy level for the pulsed ultrasonic energy induced in the ultrasonic instrument 18 as a function of a maximum ultrasonic energy level set for the ultrasonic instrument 18. More specifically, each pulsing profile 140 may be configured to induce ultrasonic energy in the ultrasonic instrument 18 that includes a series of ultrasonic energy pulses peaking at the maximum ultrasonic energy level set for the ultrasonic instrument 18 and interspaced by ultrasonic energy at a minimum ultrasonic energy level set for the ultrasonic instrument 18. The maximum ultrasonic energy level of each ultrasonic energy pulse may correspond to vibrations in the tip 20 of a maximum amplitude and velocity, and the minimum ultrasonic energy level may correspond to vibrations in the tip 20 of minimum amplitude and velocity. The maximum ultrasonic energy level may be set by the practitioner, such as to a level sufficient for ablating target tissue, and the minimum ultrasonic energy level may be specific to the pulsing profile 140 being implemented. The minimum energy factor may indicate a ratio of the maximum ultrasonic energy level set for the ultrasonic instrument 18 to use as the minimum ultrasonic energy level, and may differ among the pulsing profiles 140. Hence, given a set maximum ultrasonic energy level, each pulsing profile 140 may be configured to induce ultrasonic energy pulses in the ultrasonic instrument 18 that peak at the maximum ultrasonic energy level and are interspaced by ultrasonic energy at a different minimum ultrasonic energy level that is specific to the pulsing profile 140.

The pulse shape of each pulsing profile 140 may define the shape for a dynamic portion (also referred to as a “transitional ultrasonic energy period”) of each cycle of the pulsed ultrasonic energy induced according to the pulsing profile 140. In particular, each ultrasonic energy pulse induced in the ultrasonic instrument 18 may be defined by a transition of ultrasonic energy from a minimum ultrasonic energy level set for the ultrasonic instrument 18 to a maximum ultrasonic energy level set for the ultrasonic instrument 18, and thereafter a transition from the maximum ultrasonic energy level back to the minimum ultrasonic energy level. The period of each cycle of the induced ultrasonic energy in which the ultrasonic energy is transitioning between the minimum and maximum ultrasonic energy levels may be referred to as the dynamic portion of the cycle, and may be defined by the pulse shape of the currently selected pulsing profile 140. In other words, rather than the transitions between the maximum and minimum ultrasonic energy levels being arbitrarily shaped by the inherent electrical characteristics of the ultrasonic tool system 12, such transitions may be particularly controlled to follow a predefined transition function corresponding to the pulse shape of the applied pulsing profile 140. As examples, the pulse shape of a given pulsing profile 140 may be a hann shape corresponding to a hann wave transition function, a square shape corresponding to a square wave transition function, a triangle shape corresponding to a triangle wave transition function, a ramp up sawtooth shape corresponding to a ramp up sawtooth wave transition function, a ramp down sawtooth shape corresponding to a ramp down sawtooth shape transition function, or an inverse version of any of these pulse shapes.

The duty cycle of each pulsing profile 140 may indicate, for each cycle of pulsed ultrasonic energy induced in the ultrasonic instrument 18 according to the pulsing profile 140, a duration of the dynamic portion of the cycle relative to the total duration of the cycle. For a pulsing profile 140 with an 100% duty cycle, the dynamic portion of each cycle of the induced ultrasonic energy may extend the entire duration of the cycle. In this case, the ultrasonic energy level induced by the pulsing profile 140 may be considered as constantly transitioning. In other words, the ultrasonic energy induced by a pulsing profile 140 with an 100% duty cycle may reach the maximum and minimum ultrasonic energy levels for merely an instant (e.g., less than 1 millisecond) before transitioning to the other of the maximum and minimum ultrasonic energy levels, such as according to the pulse shape of the pulsing profile 140. Conversely, for pulsing profiles 140 associated with a duty cycle of less than 100%, the duration of the dynamic portion of each cycle of the induced ultrasonic energy may be a portion of the duration of the entire cycle that corresponds to the duty cycle. The remaining portion of each cycle, referred to as the constant portion of the cycle, may be occupied by a period of ultrasonic energy maintained at a constant level, such as the maximum or minimum ultrasonic energy levels.

The pulsing frequency of each pulsing profile 140 may indicate a frequency for the ultrasonic energy pulses induced in the ultrasonic instrument 18. While the resonant frequency of the ultrasonic instrument 18 may be between 10 and 40 kHz, the pulsing frequency may be much lower, such as less than 100 Hz. For instance, a pulsing frequency of 50 Hz for a given pulsing profile 140 would function to induce ultrasonic energy in the ultrasonic instrument 18 that includes an ultrasonic energy pulse occurring every 20 milliseconds.

As previously described, the ultrasonic energy induced in the ultrasonic instrument 18, and correspondingly the vibrations of the tip 20 of the ultrasonic instrument 18, may be proportional to the mechanical current i_{M} induced in the ultrasonic instrument 18. Each pulsing profile 140 may thus be defined in reference to a pulsing pattern for the ultrasonic energy induced in the ultrasonic instrument 18, or for the mechanical current i_{M} induced in the ultrasonic instrument 18. In other words, the maximum ultrasonic energy level set for the ultrasonic instrument 18 may be represented by a corresponding maximum mechanical current i_{M} set for the ultrasonic instrument 18, and the minimum ultrasonic energy level set for the ultrasonic instrument 18 may be represented by a corresponding minimum mechanical current i_{M} set for the ultrasonic instrument 18.

In some implementations, the control console 16 may be configured to operate the ultrasonic instrument 18 in multiple ablation modes, such as a soft tissue ablation mode for ablating soft tissue, and a hard tissue ablation mode for ablating hard tissue such as fibrous tissue and bone. In this case, the console storage 118 may be configured to store one or more distinct pulsing profiles 140 for each mode, with the pulsing parameters for each pulsing profile 140 including a parameter indicating whether the pulsing profile 140 is associated with the soft tissue ablation mode or the hard tissue ablation mode.

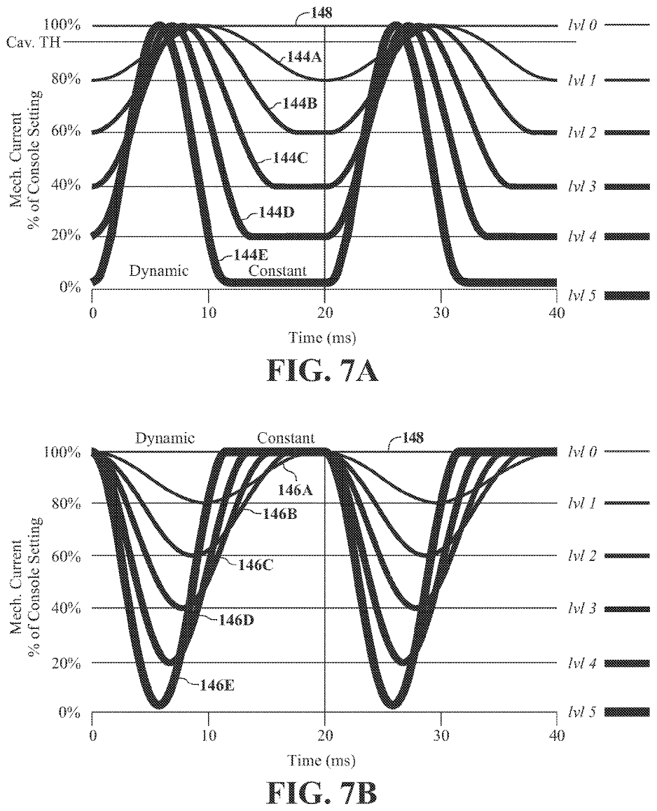

FIG. 7A illustrates pulsing patterns of soft tissue pulsing profiles 144 that may be stored by the control console 16 in association with the soft tissue ablation mode, and FIG. 7B illustrates pulsing patterns of hard tissue pulsing profiles 146 that may be stored by the control console 16 in association with the hard tissue ablation mode. In other words, responsive to determining the ultrasonic instrument 18 is set to be operated in the soft tissue ablation mode, the control console 16 may be configured to make the soft tissue pulsing profiles 144 available for user selection, and responsive to determining the ultrasonic instrument 18 is set to be operated in the hard tissue ablation mode, the control console 16 may be configured to make the hard tissue pulsing profiles 146 available for user selection. The control console 16 may be configured to determine whether the ultrasonic tool system 12 is set to operate in the soft tissue ablation mode or hard tissue ablation mode responsive to corresponding user input and/or based on data read from the ultrasonic instrument 18, as described in more detail below.

Each of FIGS. 7A and 7B also illustrates a constant energy profile 148 that may be induced in the ultrasonic instrument 18, such as when pulsing mode is disabled by the practitioner via the display 74 of the control console 16, or when the control console 16 determines that the tip 20 presently coupled to the control console 16 is not pulsing enabled.

The constant energy profile 148 illustrated in FIGS. 7A and 7B may be configured to induce ultrasonic energy in the ultrasonic instrument 18 that is maintained at a constant ultrasonic energy level, such as the maximum ultrasonic energy level set for the ultrasonic instrument 18. In other words, when the constant energy profile 148 is applied, the ultrasonic controller 112 may be configured to generate and source an AC drive signal to the ultrasonic instrument 18 that maintains the mechanical current i_{M} induced in the ultrasonic instrument 18 at a constant level, such as a constant level corresponding to the maximum ultrasonic energy level set for the ultrasonic instrument 18.

The maximum ultrasonic energy level for the ultrasonic instrument 18 may be set based on a power setting selected by the practitioner that indicates a percentage of a global ultrasonic energy limit for the ultrasonic instrument 18. The maximum ultrasonic energy level may further be adjusted down from the ultrasonic energy level indicated by the practitioner-selected power setting as a function of the position of the foot pedal 76. Specifically, the control console 16 may be configured to linearly increase the maximum ultrasonic energy level from a minimum value (e.g., zero) to the level indicated by the practitioner-selected power setting as the foot pedal 76 is moved from a fully non-depressed position to a fully depressed position. The maximum ultrasonic energy level set for the ultrasonic instrument 18 may thus vary during a procedure as a function of changes to the practitioner's power setting selection and/or the depression level of the foot pedal 76.

The 100% line illustrated in FIGS. 7A and 7B may correspond to the set maximum ultrasonic energy level. As described above, continuous operation of the ultrasonic instrument 18 at the maximum ultrasonic energy level may cause unwanted heating of the ultrasonic instrument 18 and the surgical site, and may increase potential trauma to surrounding tissue desired to remain intact.

In reference to FIG. 7A, each of the soft tissue pulsing profiles 144 may be configured to induce ultrasonic energy in the ultrasonic instrument 18 that includes a plurality of ultrasonic energy pulses interspaced by periods of ultrasonic energy at the minimum ultrasonic energy level for the ultrasonic instrument 18 determined according to the soft tissue pulsing profile 144, with each of the ultrasonic energy pulses being defined by a hann wave and peaking at the maximum ultrasonic energy level.

The dynamic periods of the pulsed ultrasonic energy induced by each soft tissue pulsing profile 144 may correspond to the periods in which the induced ultrasonic energy transitions from the minimum ultrasonic energy level to the maximum ultrasonic energy level and then back to the minimum ultrasonic energy level set according to the soft tissue pulsing profile 144. In other words, the dynamic portions for the soft tissue pulsing profiles 144 may correspond to the ultrasonic energy pulses of the induced ultrasonic energy. Accordingly, the pulse shape parameter for each of the soft tissue pulsing profiles 144 illustrated in FIG. 7A, which defines the shape of the dynamic portions of ultrasonic energy induced by the soft tissue pulsing profile 144, may be set to the hann pulse shape.

As shown in the illustrated example, the soft tissue pulsing profile 144A may have a duty cycle of 100%, and accordingly, the dynamic portion of each cycle of the ultrasonic energy induced according to the soft tissue pulsing profile 144A may extend the entirety of the cycle. Conversely, the soft tissue pulsing profiles 144B to 144E may each have a duty cycle of less than 100%. Accordingly, the dynamic portion of each cycle of the ultrasonic energy induced according to the soft tissue pulsing profiles 144B to 144E may extend only a portion of the cycle, with the remaining portion of the cycle being a constant ultrasonic energy portion in which the ultrasonic energy is maintained at the minimum ultrasonic energy level for a significant period. The duration of the constant ultrasonic energy portion may vary depending on the soft tissue pulsing profile 144 selected. For instance, the duration may be greater than or equal to two milliseconds for some soft tissue pulsing profiles 144, and greater than or equal to five milliseconds for others.

As an example, the duty cycle associated with the soft tissue pulsing profile 144B may be 90%. Assuming each soft tissue pulsing profile 144 has a pulsing frequency of 50 Hz as shown in FIG. 7A, the soft tissue pulsing profile 144B may thus be configured to induce ultrasonic energy pulses that are each 18 milliseconds in duration and interspaced by constant ultrasonic energy periods at the minimum ultrasonic energy level that are each 2 milliseconds in duration. As a further example, the duty cycle associated with the soft tissue pulsing profile 144C may be 80%. Assuming a pulsing frequency of 50 Hz, the soft tissue pulsing profile 144C may thus be configured to induce ultrasonic energy pulses in the ultrasonic instrument 18 that are each 16 milliseconds in duration and interspaced by constant ultrasonic energy periods at the minimum ultrasonic energy level that are each 4 milliseconds in duration. As another example, the duty cycle associated with the soft tissue pulsing profile 144E may be 50%. Assuming a pulsing frequency of 50 Hz, the soft tissue pulsing profile 144E may thus be configured to induce ultrasonic energy pulses in the ultrasonic instrument 18 that are each 10 milliseconds in duration and interspaced by constant ultrasonic energy periods at the minimum ultrasonic energy level that are each 10 milliseconds in duration. Thus, in the examples illustrated in FIG. 7A, the duration of each significant period of ultrasonic energy maintained at the minimum ultrasonic energy level may be greater than or equal 2 milliseconds (e.g., greater than or equal to 4 milliseconds, greater than or equal to 10 milliseconds).

As illustrated in FIG. 7A, each soft tissue pulsing profile 144 may also include a varying factor for determining the minimum ultrasonic energy level for the ultrasonic instrument 18 relative to the maximum ultrasonic energy level set for the ultrasonic instrument 18. For instance, the factor for the soft tissue pulsing profile 144A may be 80%, indicating that when the soft tissue pulsing profile 144A is selected, the minimum ultrasonic level for the ultrasonic instrument 18 should be set to a value that is 80% of the maximum ultrasonic energy level. Conversely, the factor for the soft tissue pulsing profile 144C may be 40%, indicating that when the soft tissue pulsing profile 144C is selected, the minimum ultrasonic level for the ultrasonic instrument 18 should be set to 40% of the maximum ultrasonic energy level.

In reference to FIG. 7B, each of the hard tissue pulsing profiles 146 may be configured to induce ultrasonic energy in the ultrasonic instrument 18 that includes a plurality of ultrasonic energy pulses interspaced by ultrasonic energy at the minimum ultrasonic energy level for the ultrasonic instrument 18 determined according to the hard tissue pulsing profile 146, with each of the ultrasonic energy pulses peaking at the maximum ultrasonic energy level set for the ultrasonic instrument 18. Conversely to the soft tissue pulsing profiles 144, the dynamic periods of the pulsed ultrasonic energy induced by each hard tissue pulsing profile 146 may correspond to the periods in which the induced ultrasonic energy transitions from the maximum ultrasonic energy level set for the ultrasonic instrument 18 to the minimum ultrasonic energy level set according to the hard tissue pulsing profile 146 and back to the maximum ultrasonic energy level. In other words, the dynamic portions of the hard tissue pulsing profiles 146 may correspond to the adjoining edges of each pair of adjacent ultrasonic energy pulses of the induced ultrasonic energy. Accordingly, the pulse shape parameter for each of the hard tissue pulsing profiles 146 illustrated in FIG. 7B, which may define the shape of the dynamic portions of ultrasonic energy induced by the hard tissue pulsing profile 146, may be set to the inverse hann pulse shape, corresponding to an inverse hann wave for the dynamic portions. Alternatively, because the hard tissue pulsing profiles 146 are each associated with the hard tissue ablation mode, the pulse shape for each of the hard tissue pulsing profiles 146 may indicate a non-inverted version of the desired shape (e.g., hann shape), and the ultrasonic controller 112 may be configured to inverse the shape when inducing the pulsed ultrasonic energy based on the control console 16 being set to operate in the hard tissue ablation mode.

As shown in the illustrated example, the hard tissue pulsing profile 146A may have a duty cycle of 100%, and accordingly, the dynamic portion of each cycle of the ultrasonic energy induced according to the hard tissue pulsing profile 146A may extend the entirety of the cycle. Conversely, the hard tissue pulsing profiles 146A to 146E may each have a duty cycle of less than 100%. Accordingly, the dynamic portion of each cycle of the ultrasonic energy induced according to the hard tissue pulsing profiles 146B to 146E may extend only a portion of the cycle, with the remaining portion of the cycle being a constant ultrasonic energy portion in which the ultrasonic energy is maintained at the set maximum ultrasonic energy level for a significant period. In other words, the constant ultrasonic energy periods induced by each hard tissue pulsing profile 146 associated with a duty cycle of less than 100% (e.g., pulsing profiles 146B to 146E) may correspond to the periods in which the ultrasonic energy is maintained at the maximum ultrasonic energy level at the peak of each pulse. The duration of the constant ultrasonic energy portions may vary depending on the hard tissue pulsing profile 146 selected. For instance, the duration may be greater than or equal to two milliseconds for some hard tissue pulsing profiles 146, and greater than or equal to five milliseconds for others.

As an example, the duty cycle associated with the hard tissue pulsing profile 146B may be 90%. Assuming each hard tissue pulsing profile 146 has a pulsing frequency of 50 Hz as shown in FIG. 7B, the pulsing profile 146B may thus be configured to induce ultrasonic energy in which the adjoining edges of each pair adjacent pulses are 18 milliseconds in duration, and each ultrasonic energy pulse peaks at the maximum ultrasonic energy level for 2 milliseconds in duration. As a further example, the duty cycle for the pulsing profile 146C may be 80%. Assuming a pulsing frequency of 50 Hz, the pulsing profile 146C may thus be configured to induce ultrasonic energy in the ultrasonic instrument 18 including ultrasonic energy pulses each peaking and including a period of ultrasonic energy maintained at the maximum ultrasonic energy level set for the ultrasonic instrument 18 that is 4 milliseconds in duration, with the constant ultrasonic energy periods being interspaced by dynamic ultrasonic energy periods that are each 16 milliseconds in duration. As another example, the duty cycle for the pulsing profile 146E may be 50%. Assuming a pulsing frequency of 50 Hz, the pulsing profile 146E may thus be configured to induce ultrasonic energy in the ultrasonic instrument 18 including ultrasonic energy pulses each peaking and including a constant ultrasonic energy period at the maximum ultrasonic energy level set for the ultrasonic instrument 18 that is 10 milliseconds in duration, with the constant ultrasonic energy periods being interspaced by dynamic ultrasonic energy periods that are each 10 milliseconds in duration. Thus, in the examples illustrated in FIG. 7B, the duration of each significant period of ultrasonic energy maintained at the maximum ultrasonic energy level may be greater than or equal 2 milliseconds (e.g., 4 milliseconds, 10 milliseconds).

As illustrated in FIG. 7B, each hard tissue pulsing profile 146 may also include a varying factor for determining the minimum ultrasonic energy level for the ultrasonic instrument 18 relative to the maximum ultrasonic energy level set for the ultrasonic instrument 18. For instance, the factor for the hard tissue pulsing profile 146A may be 80%, indicating that when the hard tissue pulsing profile 146A is selected, the minimum ultrasonic level for the ultrasonic instrument 18 should be set to a value that is 80% of the maximum ultrasonic energy level. Conversely, the factor for the hard tissue pulsing profile 146C may be 40%, indicating that when the pulsing profile 146C is selected, the minimum ultrasonic level for the ultrasonic instrument 18 should be set to a value that is 40% of the maximum ultrasonic energy level.

The varying pulsing profiles 144, 146 may provide varying operating characteristics, such as varying levels of tissue selectivity, temperature control, and tactile feedback. The preferred level of such operating characteristics may depend on the personal preferences of the practitioner and on the type of tissue being targeted for ablation. The level of such operating characteristics provided by each pulsing profile 144, 146 may be a function of the duty cycle, minimum ultrasonic energy level, and pulsing frequency of the pulsing profile 144, 146.

For instance, each of the soft tissue pulsing profiles 144 illustrated in FIG. 7A has a different factor for determining the minimum ultrasonic energy level and a different duty cycle. Assuming other pulsing parameters were to remain constant among the soft tissue pulsing profiles 144, the lower the minimum ultrasonic energy level defined by a given soft tissue pulsing profile 144 relative to another soft tissue pulsing profile 144, the lower the average amplitude and velocity of the vibrations of the tip 20 that may be induced by the given soft tissue pulsing profile 144. Similarly, assuming other pulsing parameters were to remain constant among the soft tissue pulsing profiles 144, the lower the duty cycle of a given soft tissue pulsing profile 144 relative to another soft tissue pulsing profile 144, the lower the average amplitude and velocity of the vibrations of the tip 20 that may be induced by the given pulsing profile 144.

The lower the average amplitude and velocity of the vibrations of the tip 20 that are induced by a given soft tissue pulsing profile 144, the less effective the vibrations of the tip 20 may be at resecting firmer tissues, thereby providing increased tissue selectivity, and the less heat that may be generated by the ultrasonic instrument 18 when resecting tissue. In other words, the lower the average amplitude and velocity of the vibrations of the tip 20 that are induced by a given soft tissue pulsing profile 144, the greater the ratio of tissue preservation of non-targeted firmer tissue verses the resection rate of softer target tissue that may be provided.

Moreover, assuming other pulsing parameters were to remain constant among the soft tissue pulsing profiles 144, the lower the minimum ultrasonic energy level defined by a given soft tissue pulsing profile 144 relative to another soft tissue pulsing profile 144, the more tactile feedback that may be felt by the practitioner holding the ultrasonic instrument 18 from the ultrasonic energy induced in the ultrasonic instrument 18 according to the given soft tissue pulsing profile 144. Similarly, assuming other pulsing parameters were to remain constant among the soft tissue pulsing profiles 144, the lower the duty cycle defined by a given soft tissue pulsing profile 144 relative to another soft tissue pulsing profile 144, the more tactile feedback that may be felt by the practitioner holding the ultrasonic instrument 18 from the ultrasonic energy induced in the ultrasonic instrument 18 according to the given soft tissue pulsing profile 144.

As illustrated in FIG. 7A, each of the soft tissue pulsing profiles 144 may be associated with a different pulse control level (e.g., lvl 1 to lvl 5) that may be selected by the practitioner, such as using the display 74 of the control console 16, to cause the control console 16 to induce ultrasonic energy in the ultrasonic instrument 18 according to the soft tissue pulsing profile 144. In some examples, the pulse control levels may be assigned to the soft tissue pulsing profiles 144 such that each incremental pulse control level is associated with a soft tissue pulsing profile 144 that offers increased tissue selectivity, increased temperature control, and/or increased tactile feedback. More particularly, as illustrated in FIG. 7A, each selectable pulse control level may be associated with a soft tissue pulsing profile 144 defining a lower minimum ultrasonic energy level and/or duty cycle than the soft tissue pulsing profile 144 associated with the preceding selectable pulse control level. Selectable pulse control levels lower in the order may thus be associated with soft tissue pulsing profiles 144 configured for ablating more tissue types, or more particularly firmer tissues, than those associated with pulse control levels higher in the order. Selectable pulse control levels lower in the order may also be associated with soft tissue pulsing profiles 144 configured for providing less tactile feedback than those higher in the order.

Ordering the soft tissue pulsing profiles 144 in this manner may offer an intuitive means by which a practitioner may select a soft tissue pulsing profile 144 that corresponds to the practitioner's desired operating characteristics. Specifically, a practitioner may request increased tissue selectivity and temperature control, and/or may request increased tactile feedback, of the ultrasonic instrument 18 by selecting a relatively higher pulse control level, and may request decreased tissue selectivity and temperature control, and/or may request decreased tactile feedback, by selecting a relatively lower pulse control level.

Each of the hard tissue pulsing profiles 146 illustrated in FIG. 7B likewise define a different minimum energy factor and duty cycle. Assuming other pulsing parameters were to remain constant among the hard tissue pulsing profiles 146, the lower the minimum ultrasonic energy level defined by a given hard tissue pulsing profile 146 relative to another hard tissue pulsing profile 146, the lower the average amplitude and velocity of the vibrations of the tip 20 that may be induced by the given hard tissue pulsing profile 146. The lower the average and amplitude and velocity of the vibrations of the tip 20 induced by a given hard tissue pulsing profile 146, the more temperature control that may be provided by the given pulsing profile 146.

Furthermore, assuming other pulsing parameters were to remain constant among the hard tissue pulsing profiles 146, the lower the minimum ultrasonic energy level defined by a given hard tissue pulsing profile 146 relative to another hard tissue pulsing profile 146, the more tactile feedback that may be provided to the practitioner from the ultrasonic energy induced in the ultrasonic instrument 18 according to the hard tissue pulsing profile 146, such as upon placing the vibrating tip 20 against different tissues. Similarly, assuming other pulsing parameters were to remain constant among the hard tissue pulsing profiles 146, the lower the duty cycle defined by a given hard tissue pulsing profile 146 relative to another hard tissue pulsing profile 146, the more tactile feedback that may be provided to the practitioner from the ultrasonic energy induced in the ultrasonic instrument 18 according to the hard tissue pulsing profile 146, such as upon placing the vibrating tip 20 against different tissues.

The more tactile feedback that may be felt by the practitioner, the more the practitioner may be able to feel the ultrasonic energy pulses, which may encourage the practitioner to make back and forth motions with the ultrasonic instrument 18 that are often desirable when cutting hard tissue. In addition, because certain tissues, such as soft tissues, may provide increased dampening of the vibrations felt by the practitioner relative to other tissues, the more tactile feedback that may be felt by the practitioner, the more noticeable it may be to the practitioner when the tip 20 breaks through hard tissue or inadvertently contacts soft tissue during a procedure. Tactile feedback may also be used to indicate to the practitioner that a preferred amount of force is being applied to the ultrasonic instrument 18 by the practitioner, as described in more detail below.

In addition, assuming other pulsing parameters were to remain constant among the hard tissue pulsing profiles 146, the lower the minimum ultrasonic energy level defined by a given hard tissue pulsing profile 146 relative to another hard tissue pulsing profile 146, the more likely the tip 20 may be to stall as the load placed on the tip 20 increases, which may help reduce undesired ablation and/or necrosis of non-targeted tissue.

As illustrated in FIG. 7B, each of the hard tissue pulsing profiles 146 may be associated with a different pulse control level (e.g., lvl 1 to lvl 5) that may be selected by the practitioner, such as using the display 74 of the control console 16, to cause the control console 16 to induce ultrasonic energy in the ultrasonic instrument 18 according to the hard tissue pulsing profile 146. In some examples, the pulse control levels may be assigned to the hard tissue pulsing profiles 146 such that each incremental pulse control level is associated with a hard tissue pulsing profile 146 that offers increased tactile feedback and/or a greater stall potential. More particularly, each selectable pulse control level may be associated with a hard tissue pulsing profile 146 defining a lower minimum ultrasonic energy level and/or lower duty cycle than the hard tissue pulsing profile 146 associated with the preceding selectable pulse control level. Selectable pulse control levels lower in the order may thus be associated with hard tissue pulsing profiles 146 configured for providing less tactile feedback than those associated with the pulse control levels higher in the order. Selectable pulse control levels lower in the order may also be associated with hard tissue pulsing profiles 146 configured for providing less potential for stalling than those higher in the order.

Ordering the hard tissue pulsing profiles 146 in this manner may offer an intuitive means by which a practitioner may select a hard tissue pulsing profile 146 that corresponds to the practitioner's desired operating characteristics. For instance, a practitioner may request greater levels of tactile feedback and/or stall potential by selecting a higher pulse control level in the order, and may request decreased levels of tactile feedback and/or stall potential by selecting a lower pulse control level in the order.

Further referring to the hard tissue pulsing profiles 146 illustrated in FIG. 7B, assuming other pulsing parameters were to remain constant among the hard tissue pulsing profiles 146, the lower the minimum ultrasonic energy level defined by a given hard tissue pulsing profile 146 relative to another hard tissue pulsing profile 146, the lower the average displacement and velocity of the tip 20 that may be induced by the given hard tissue pulsing profile 146. Such lower average displacement and velocity may reduce the resection rate of the ultrasonic instrument 18 when operating against hard tissue. To help maintain a desired resection rate, as further illustrated in FIG. 7B, each incremental pulse control level may also be associated with a hard tissue pulsing profile 146 that defines a decreased duty cycle relative to the hard tissue pulsing profile 146 associated with the preceding pulse control level. This configuration may function to increase the period in which each hard tissue pulsing profile 146 induces ultrasonic energy maintained at the maximum ultrasonic energy level set for the ultrasonic instrument 18, and correspondingly may increase the average displacement and velocity of the tip 20 induced by the hard tissue pulsing profile 146.

Each of the pulsing profiles 144, 146 illustrated in FIGS. 7A and 7B have a similar pulsing frequency, namely 50 Hz. In alternative examples, two or more of the soft tissue pulsing profiles 144 defined by the ultrasonic tool system 12 may have varying pulsing frequencies, and two or more of the hard tissue pulsing profiles 146 defined by the ultrasonic tool system 12 may likewise have varying pulsing frequencies.

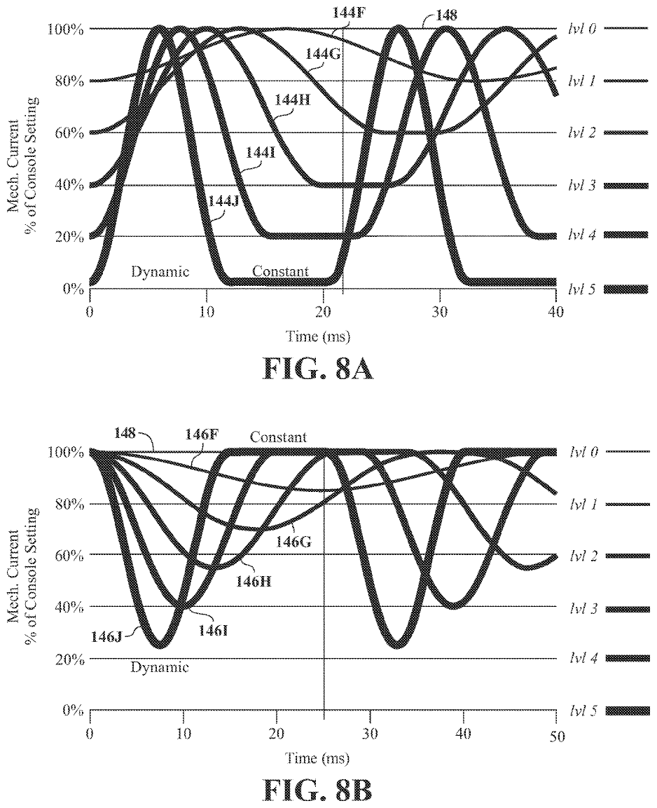

As an example, FIG. 8A illustrates soft tissue pulsing profiles 144F to 144J each associated with a different selectable pulse control level (lvl 1 to lvl 5) such that the soft tissue pulsing profile 144 associated with each incremental pulse control level provides a lower minimum ultrasonic energy level, lower duty cycle, and greater pulsing frequency than the soft tissue pulsing profile 144 associated with the preceding pulse control level. For instance, the soft tissue pulsing profiles 144F to 144J may have pulsing frequencies of 30 Hz, 35 Hz, 45 Hz, 50 Hz, and 55 Hz respectively. Assuming other pulsing parameters were to remain constant among the soft tissue pulsing profiles 144, the greater the pulsing frequency defined by a given soft tissue pulsing profile 144 relative to another soft tissue pulsing profile 144, the more tactile feedback that may be felt by the practitioner holding the ultrasonic instrument 18 from the ultrasonic energy induced in the ultrasonic instrument 18 according to the given soft tissue pulsing profile 144. Accordingly, in addition to each incremental tissue pulsing profile 144 providing increased tissue selectivity and temperature control, the difference in the level of tactile feedback provided between each pair of adjacent soft tissue pulsing profiles 144 of FIG. 8A may be greater than that of the corresponding pair of adjacent soft tissue pulsing profiles 144 of FIG. 7A.

As a further example, FIG. 8B illustrates hard tissue pulsing profiles 146F to 146J each associated with a different selectable pulse control level (lvl 1 to lvl 5) such that the hard tissue pulsing profile 146 associated with each incremental pulse control level has a lower minimum ultrasonic energy level, lower duty cycle, and greater pulsing frequency than the hard tissue pulsing profile 146 associated with the preceding pulse control level. For instance, the hard tissue pulsing profiles 146F to 146J may have pulsing frequencies of 20 Hz, 25 Hz, 30 Hz, 35 Hz, and 40 Hz respectively. Assuming other pulsing parameters were to remain constant among the hard tissue pulsing profiles 146, the greater the pulsing frequency defined by a given hard tissue pulsing profile 146 relative to another hard tissue pulsing profile 146, the more tactile feedback that may be felt by the practitioner holding the ultrasonic instrument from the ultrasonic energy induced in the ultrasonic instrument 18 according to the given hard tissue pulsing profile 146. Accordingly, in addition to each incremental hard tissue pulsing profile 146 providing a greater stall potential, the difference in the level of tactile feedback provided between each pair of adjacent hard tissue pulsing profiles 146 of FIG. 8B may be greater than that of the corresponding pair of adjacent hard tissue pulsing profiles 146 of FIG. 7B.

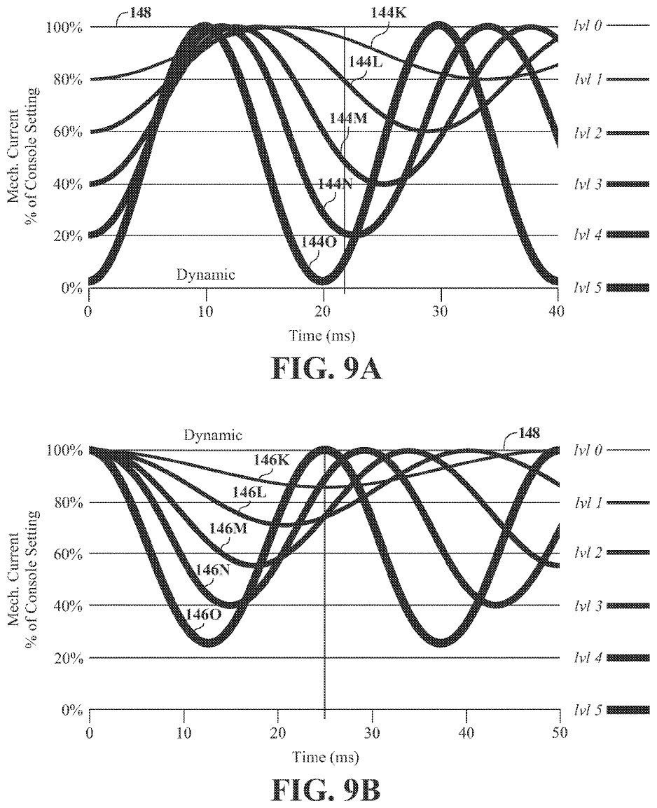

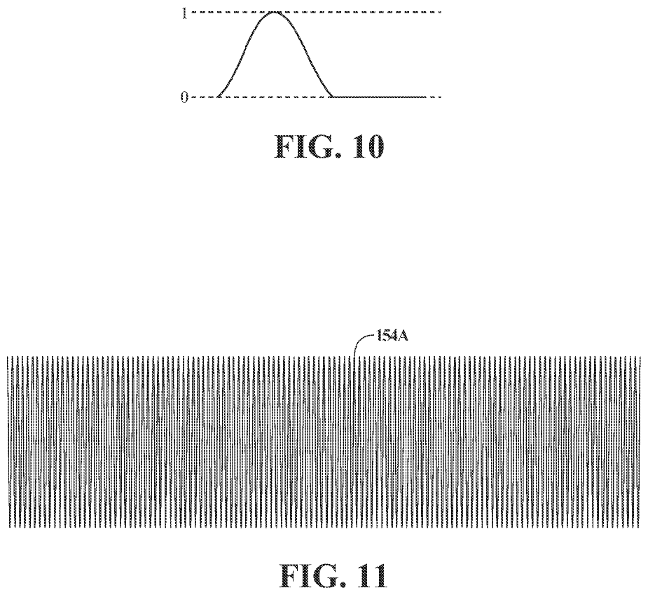

As shown in the previous examples, the varying pulsing profiles 144, 146 defined by the ultrasonic tool system 12 may include varying duty cycles. In alternative examples, the ultrasonic tool system 12 may be configured to implement pulsing profiles 144, 146 having a same duty cycle. For instance, FIG. 9A illustrates soft tissue pulsing profiles 144K-144O each associated with a different selectable pulse control level (lvl 1 to lvl 5) such that the soft tissue pulsing profile 144 associated with each incremental pulse control level has a lower minimum ultrasonic energy level, a greater pulsing frequency, and a same duty cycle (e.g., 100%) as the soft tissue pulsing profile 144 associated with the proceeding pulse control level. In this case, each incremental soft tissue pulsing profile 144 may continue to provide increased tissue selectivity, temperature control, and tactile feedback, but to an extent that is less than the corresponding soft tissue pulsing profile 144 illustrated in FIG. 8A. As a corollary, each of the soft tissue pulsing profiles 144 illustrated in FIG. 8A with a duty cycle of less than 100% may have a resection rate less than the corresponding soft tissue pulsing profile 144 illustrated in FIG. 9A.

Similarly, FIG. 9B illustrates hard tissue pulsing profiles 146K-146O each associated with a different pulse control level (lvl 1 to lvl 5) such that the hard tissue pulsing profile 146 associated with each incremental pulse control level has a lower minimum ultrasonic energy level, a greater pulsing frequency, and a same duty cycle (e.g., 100%) as the hard tissue pulsing profile 146 associated with the preceding pulse control level. In this case, each incremental hard tissue pulsing profile 146 may continue to provide increased tactile feedback and stall potential, but the extent of tactile feedback provided by each hard tissue pulsing profile 146 may be less than that of the corresponding hard tissue pulsing profile 146 illustrated in FIG. 8B. As a corollary, each of the hard tissue pulsing profiles 146 illustrated in FIG. 8B with a duty cycle of less than 100% may have a resection rate greater than the corresponding hard tissue pulsing profile 146 illustrated in FIG. 9B, which may provide increased cutting and temperature control relative to the corresponding hard tissue pulsing profile 146 of FIG. 8B.

In some implementations, the ultrasonic tool system 12 may store a set of soft tissue pulsing profiles 144 and a set of hard tissue pulsing profiles 146, with the pulsing profiles of both sets varying by the same pulsing parameters with respect to each other. For instance, the stored soft tissue pulsing profiles 144 may vary in minimum energy factor, duty cycle, and pulsing frequency (e.g., FIG. 8A), and the stored hard tissue pulsing profiles 146 may similarly vary by the same pulsing parameters (e.g., FIG. 8B). Alternatively, the pulsing parameters by which the set of soft tissue pulsing profiles 144 vary may differ from the pulsing parameters by which the set of hard tissue pulsing profiles 146 vary. As an example, the soft tissue pulsing profiles 144 may vary by minimum energy factor, duty cycle, and pulsing frequency (e.g., FIG. 8A), and the hard tissue pulsing profiles 146 may vary by minimum energy factor and pulsing frequency but not duty cycle (e.g., FIG. 9B). In other words, the set of soft tissue pulsing profiles 144 of any one of FIGS. 7A, 8A, and 9A may be stored within and implemented by the ultrasonic tool system 12 with the hard tissue pulsing profiles 146 of any one of FIGS. 7B, 8B, and 9B.

Some of the above exemplary pulsing profiles 144, 146, such as those at a highest pulse control level, may have a minimum energy factor that corresponds to a minimum ultrasonic energy level close to zero, such as pulsing profiles 144E, 146E, 144J, and 144O of FIGS. 7A, 7B, 8A, and 9A respectively. However, as shown in the illustrated examples, the minimum energy factor of these pulsing profiles 144, 146 may be set so that the minimum ultrasonic energy level does not reduce all the way to zero, but instead just above zero. This may be done so that the ultrasonic energy induced in the ultrasonic instrument 18 does not reach a level in which the control console 16 is unable to track the resonant frequency of the ultrasonic instrument 18. In other words, the minimum ultrasonic level defined by these pulsing profiles 144, 146 may be set so that the vibrations induced in the tip 20 have a magnitude sufficient for the control console 16 to detect the vibrations and track the resonant frequency of the ultrasonic instrument 18. For instance, the minimum energy factor for these pulsing profiles 144, 146 may be set so that the peak-to-peak vibrations of the tip head 22 corresponding to the minimum ultrasonic energy level is greater than 5 microns and less than 20 microns, such about 10 microns. Said differently, the minimum energy factor for these pulsing profiles 144, 146 may be set to 3% or greater, and/or so that the minimum mechanical current i_{M} induced in the ultrasonic instrument 18 is greater than 2 milliamps and less than 10 milliamps, such as approximately 5 milliamps.

Responsive to selection of a given pulsing profile 140, the ultrasonic controller 112 may be configured to cause the control console 16 to generate and source an AC drive signal to the ultrasonic instrument 18 that induces ultrasonic energy in the ultrasonic instrument 18 according to the selected pulsing profile 140. Specifically, referring again to FIG. 4, the ultrasonic controller 112 may be configured to communicate one or more control signals to the signal generator 114 that causes the signal generator 114 to generate an AC signal across the primary winding 124 that corresponds to the selected pulsing profile 140, or more particularly, that induces an AC drive signal across the secondary winding 126, which in turn induces ultrasonic energy according to the selected pulsing profile 140 in the ultrasonic instrument 18.

For instance, responsive to receiving selection of a given pulsing profile 140, the ultrasonic controller 112 may be configured to retrieve the pulsing profile 140, or more particularly the pulsing parameter settings of the pulsing profile 140, from the console storage 118. The ultrasonic controller 112 may also be configured to generate and store a modulation waveform corresponding to the retrieved pulsing profile 140, such as in a modulation DDS 150 of the signal generator 114. The modulation DDS 150 may include a memory device for storing a sample array populated with values forming the modulation waveform. The modulation waveform may extend between zero and one inclusive, and may have a shape and length corresponding of one cycle of the pulsing pattern represented by the selected pulsing profile 140.

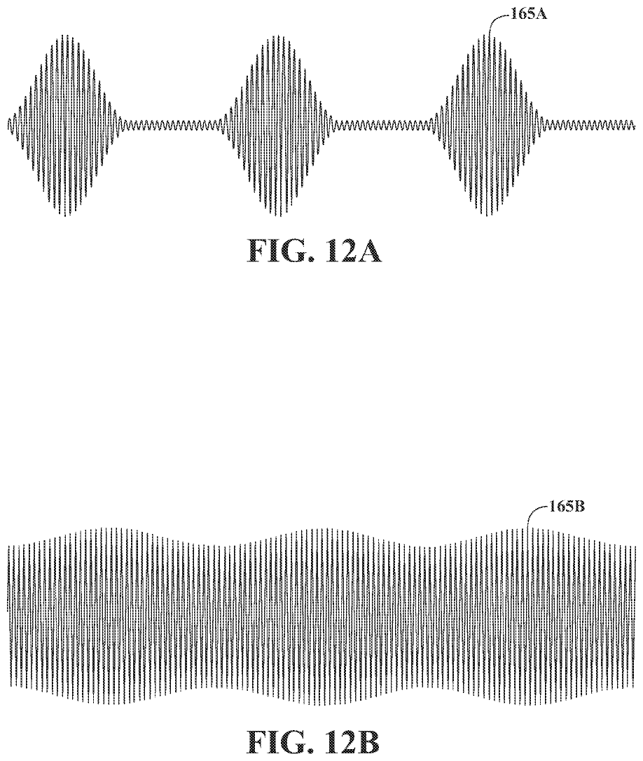

More specifically, the modulation waveform may include an instance of the transition function associated with the pulse shape parameter setting for the selected pulsing profile 140 that extends from zero and peaks at one. If the selected pulsing profile 140 has an 100% duty cycle, then the transition function may extend the entirety of the modulation waveform. If not, then the transition function may extend along a portion of the modulation waveform such that the length of the transition function relative to the length of the modulation waveform corresponds to the duty cycle. In this case, the remaining portion of the modulation waveform may be a constant period maintained at a constant value, such as zero or one. For instance, if the selected pulsing profile 140 is a soft tissue pulsing profile 144 illustrated in FIG. 7A, 8A, or 9A, then the remaining portion may be set to zero, and if the selected pulsing profile 140 is a hard tissue pulsing profile 146 illustrated in FIG. 7B, 8B, or 9B, then the remaining portion may be set to one. As an example, FIG. 10 illustrates a modulation waveform that may generated and stored by the ultrasonic controller 112 upon selection of soft tissue pulsing profile 144E illustrated in FIG. 7A.

Responsive to actuation of the ultrasonic instrument 18, such as via a depression of the foot pedal 76, the ultrasonic controller 112 may be configured to communicate a target ultrasonic frequency to the signal generator 114, or more particularly to a base DDS 152 of the signal generator 114. The base DDS 152 may store a sample array forming a sinusoidal waveform having a frequency greater than or equal to a maximum ultrasonic frequency that can be sourced to the ultrasonic instrument 18. From this sample array, the base DDS 152 may be configured to generate a base AC signal 154. The base AC signal 154 may be a sinusoidal signal with a frequency equal to the target ultrasonic frequency indicated by the ultrasonic controller 112, and may have a substantially constant amplitude, such as one.

Initially, the target ultrasonic frequency communicated by the ultrasonic controller 112 may be a predefined target frequency, which may have been read from the ultrasonic instrument 18 as described in more detail below. Thereafter, the ultrasonic controller 112 may be configured to implement a loop for tracking the frequency corresponding to a target vibratory characteristic of the ultrasonic instrument 18 (e.g., resonance), as described above, and communicate a control signal to the base DDS 152 that regulates the frequency of the base AC signal 154 generated by the base DDS 152 according to the tracked frequency. FIG. 11 illustrates a base AC signal 154A that may be generated by the base DDS 152.

Further upon actuation of the ultrasonic instrument 18, the ultrasonic controller 112 may be configured to determine the maximum and minimum ultrasonic energy levels for the induced ultrasonic energy, as described above. The ultrasonic controller 112 may then be configured to implement a loop for regulating the magnitude of the ultrasonic energy induced in the ultrasonic instrument 18 according to the selected pulsing profile 140, such as by regulating the mechanical current i_{M} induced in the ultrasonic instrument 18 according to the selected pulsing profile 140. Iterations of the loop may function to determine a target ultrasonic energy waveform for the induced ultrasonic energy based on the maximum and minimum ultrasonic energy levels, and generate an AC drive signal based on the target ultrasonic energy waveform. Specifically, the ultrasonic controller 112 may be configured to determine a scalar based on the determined maximum ultrasonic energy level and the determined minimum ultrasonic energy level, multiply the modulation waveform by the scalar, and add the determined minimum ultrasonic energy level to the result of the multiplication to generate the target ultrasonic energy waveform. The ultrasonic controller 112 may then be configured to compare the target ultrasonic energy waveform to the ultrasonic energy being induced in the ultrasonic instrument 18 to determine an error therebetween between, and adjust the base AC signal 154 with scalars 156 so as to minimize the error, such as using a PID controller.

At a more granular level, for each iteration of the loop, the ultrasonic controller 112 may be configured to determine a target ultrasonic energy level for the ultrasonic instrument 18, such as in the form of a target mechanical current i_{M} value, based on the maximum and minimum ultrasonic energy levels for the induced ultrasonic energy. In particular, the ultrasonic controller 112 may be configured to determine a scalar based on the maximum and minimum ultrasonic energy level, such as by determining a difference therebetween. The ultrasonic controller 112 may then be configured to retrieve a sample from the sample array of the modulation DDS 150, and multiply the modulation waveform sample by the scalar. Thereafter, the ultrasonic controller 112 may subtract the minimum ultrasonic energy level from the product of the multiplication to generate the target ultrasonic energy level, or more particularly the target mechanical current i_{M} value, for the ultrasonic instrument 18.

Contemporaneously with determining a target ultrasonic energy level, the ultrasonic controller 112 may be configured to determine the ultrasonic energy level being induced in the ultrasonic instrument 18, such as by calculating a mechanical current i_{M} value based on feedback data corresponding to the sourced AC drive signal as described above. The ultrasonic controller 112 may then be configured to compare and determine an error between the target ultrasonic energy level and the determined ultrasonic energy level being induced in the ultrasonic instrument 18, and generate a voltage scalar 156 that, when multiplied by the base AC signal 154, minimizes the error, such as using a PID controller.

For each iteration of the loop, the ultrasonic controller 112 may pull a sample value from the modulation waveform sample array according to the order of the samples within the array. The sample rate in which the ultrasonic controller 112 pulls sample values from the modulation waveform sample array may depend on the size of the modulation waveform array relative to the pulsing frequency of the selected pulsing profile 140 and the time of each iteration of the loop, which in one example may be 400 microseconds. For instance, if the size of the modulation waveform sample array multiplied by the loop time equals the period represented by the pulsing frequency, then for each iteration of the loop, the ultrasonic controller 112 may pull the sample value immediately following the previously pulled sample value within the modulation waveform array. Conversely, if the size of the modulation waveform sample array multiplied by the loop time is greater than the period represented by the pulsing frequency, then the ultrasonic controller 112 may pull samples at a relatively faster sampling rate, such as by skipping samples in the array (e.g., pulling every fifth sample). As a further example, if the size of the modulation waveform sample array multiplied by the loop time is less than the period represented by the pulsing frequency, then the ultrasonic controller 112 may pull samples at a relatively slower sampling rate, such as by using a given sample for multiple iterations of the loop. As explained in more detail below, the ultrasonic controller 112 may be configured to adjust the pulsing frequency during a procedure, such as a function of the load being applied to the ultrasonic instrument 18, which in turn may cause the ultrasonic controller 112 to adjust the sampling rate.

As previously mentioned, the maximum ultrasonic energy level, and correspondingly the minimum ultrasonic energy level, for the induced pulsed ultrasonic energy may vary during a procedure, such as a result of the practitioner adjusting the set power level and/or depression level of the foot pedal 76. It should be appreciated that the above algorithm enables the control console 16 to account for such variation without altering the modulation waveform stored in the modulation DDS 150, thereby improving the responsiveness of the system.

The signal generator 114 may further include a multiplier 158 configured to receive and multiply the base AC signal 154 with the generated scalars 156 to generate a modulated AC signal 160. The modulated AC signal 160 may be communicated to a D/A converter 162 and then through an amplifier 164, which may receive a power signal from a power supply 165 regulated by the ultrasonic controller 112. The amplifier 164 may generate a corresponding AC signal across the primary winding 124 of the transformer 116. As one example, the amplifier 164 and power supply 165 may be configured as described in Applicant's U.S. Pat. No. 10,449,570, the contents of which are hereby incorporated by reference herein in their entirety.

The AC signal across the primary winding 124 may induce an AC drive signal across the secondary winding 126 that in turn induces ultrasonic energy in the ultrasonic instrument 18 according to the selected pulsing profile 140. In other words, the AC signal across the primary winding 124 may induce an AC drive signal across the secondary winding 126 that in turn may induce ultrasonic energy in the ultrasonic instrument 18 including a plurality of ultrasonic energy pulses, each of the pulses peaking at the maximum ultrasonic energy level determined for the ultrasonic instrument 18 and being interspaced by ultrasonic energy at the minimum ultrasonic energy level defined according to the selected pulsing profile 140. FIG. 12A illustrates an AC signal 165A that may be generated across the primary winding 124 by the signal generator 114, such as upon selection of the soft tissue pulsing profile 144E shown in FIG. 7A. FIG. 12B illustrates another AC signal 165B that may be generated across the primary winding 124 by the signal generator 114, such as upon selection of the soft tissue pulsing profile 144A shown in FIG. 7B.

In alternative implementations, the base DDS 152 may be configured to generate the base AC signal 154 so as to have the tracked ultrasonic frequency indicated by the ultrasonic controller 112 and an amplitude corresponding to the maximum ultrasonic energy level set for the ultrasonic instrument 18. In particular, the ultrasonic controller 112 may be configured to implement a loop of determining an error between the determined maximum ultrasonic energy level and a measured ultrasonic energy level induced in the ultrasonic instrument, and provide scalars to the base DDS 152 that minimize the error. In this case, the modulation waveform generated and stored in the modulational DDS 150 may extend between one and the minimum energy factor for the selected pulsing profile 140. The signal generator 114 may then be configured to multiply the base AC signal 154 with the modulation waveform to generate the modulated AC signal 160.

Referring again to FIGS. 4 and 5, the control console 16 may also include a memory reader 166 for communicating with one or more electronic memory storage devices integral with the ultrasonic instrument 18. The ultrasonic instrument 18 may include one or more electronic memory storage devices for storing data that identifies the ultrasonic instrument 18, or more particularly the handpiece 24 and/or tip 20, and defines operational parameter settings specific to the ultrasonic instrument 18, or more particularly to the handpiece 24 and/or tip 20. Non-limiting examples of such operational parameters may include a maximum drive current for the AC drive signal, a maximum current for the mechanical current i_{M}, a maximum drive voltage for the AC drive signal, a maximum drive frequency for the AC drive signal, a minimum drive frequency for the AC drive signal, a capacitance C_{O} of the drivers 30, PID coefficients for regulating the AC drive signal, a use history, and whether the ultrasonic instrument 18, or more particularly the tip 20, is pulsing enabled. The one or more memory devices integral with the ultrasonic instrument 18 may also indicate whether the tip 20 coupled to the handpiece 24 is configured for ablating soft or hard tissue, and may indicate one or more pulsing profiles 140 particular to the tip 20.

For instance, the handpiece 24 of the ultrasonic instrument 18 may include a handpiece (HP) memory 168 disposed therein. As non-limiting examples, the HP memory 168 may be an EPROM, an EEPROM, or an RFID tag. Responsive to connecting the ultrasonic instrument 18 to the control console 16, the ultrasonic controller 112 may be configured to read the data stored in the HP memory 168 using the memory reader 166, and to tailor operation of the control console 16 based on the data. More particularly, the control console 16 may include a communication interface, such as a coil 170, connected to the memory reader 166. The coil 170 may be integral with the socket 40 of the control console 16. The HP memory 168 may similarly be connected to a coil 172, which may be integral with the adapter 38 of the cable 36. When the ultrasonic instrument 18 is connected to the control console 16 via the cable 36, the coils 170, 172 may become aligned and able to inductively exchange signals. The ultrasonic controller 112 may then be configured to read data from and write data to the HP memory 168 over the coils 170, 172.

The memory reader 166 may be configured to convert signals across the coil 170 into data signals readable by the ultrasonic controller 112. The memory reader 166 may also be configured to receive data to be written to the HP memory 168 from the ultrasonic controller 112, and to generate signals across the coil 170 that causes the data to be written to the HP memory 168. The structure of the memory reader 166 may complement that of the HP memory 168. Thus, continuing with the above non-limiting examples, the memory reader 166 may be an assembly capable of reading data from and writing data to an EPROM, EEPROM, or RFID tag.

In addition or alternatively to the HP memory 168, the ultrasonic instrument 18 may include a tip memory 174. As described above, the tip 20 may be removable from the handpiece 24 so the handpiece 24 can be used with interchangeable tips 20, and different tips 20 may have different operational limitations and intended uses. For instance, some tips 20 may be configured for ablating soft tissue, and other tips 20 may be configured for ablating hard tissue such as fibrous tissue and bone. Accordingly, the HP memory 168 may store data identifying the handpiece 24 and operational parameter settings specific to the handpiece 24, including the capacitance C_{O} of the drivers 30, and the tip memory 174 may store data identifying the tip 20 currently coupled to the handpiece 24 and operational parameter settings specific to the tip 20, including whether the tip 20 is configured for ablating soft tissue or cutting hard tissue such as bone, and/or pulsing parameter settings for pulsing profiles 140 specific to the tip 20. Because the tip 20 and irrigation sleeve 42 may be distributed together as a single package, the tip memory 174 may be disposed in the irrigation sleeve 42, or more particularly the sleeve body 44. The tip memory 174 may be the same type of memory as the HP memory 168 (e.g., an EPROM, an EEPROM, or an RFID tag).

Responsive to connecting the ultrasonic instrument 18 to the control console 16, the ultrasonic controller 112 may be configured to read the data stored in the HP memory 168 and the tip memory 174 using the memory reader 166, and to tailor operation of the control console 16 to the specific handpiece 24 and tip 20 combination coupled to the control console 16. The tip memory 174 may include settings for the same operational parameters as the HP memory 168. To the extent the settings, which may also be referred to as values, for a given operational parameter differ between the HP memory 168 and the tip memory 174, the ultrasonic controller 112 may be configured to utilize the more restrictive value to manage operation of the ultrasonic instrument 18. Additionally, or alternatively, to the extent both the HP memory 168 and the tip memory 174 include a value for a same operational parameter, the ultrasonic controller 112 may be configured to manage operation of the ultrasonic instrument 18 relative to the operational parameter based on a combination of the values stored in the memories (e.g., summing the values, averaging the values).

Similar to the HP memory 168, the ultrasonic controller 112 may read data from and write data to the tip memory 174 via the memory reader 166 and coil 170. In particular, the handpiece 24 may include two conductors 176 extending from the proximal end to the distal end of the handpiece 24. The proximal ends of the conductors 176 may be coupled to the coil 172, which may be integral with the adapter 38 of the cable 36. The distal ends of the conductors 176 may be coupled to another coil 178 disposed at the distal end of the handpiece 24. A corresponding coil 180 may be disposed in a proximal end of the irrigation sleeve 42, or more particularly the sleeve body 44. When the irrigation sleeve 42 is disposed around the tip 20 and fitted to the handpiece 24, the coils 178, 180 may become aligned and able to inductively exchange signals. When the handpiece 24 is then connected to the control console 16 via the cable 36, the coils 170, 172 may also become aligned and able to inductively exchange signals. The ultrasonic controller 112 may then read data from and write data to the tip memory 174 over the conductors 176 via inductive communication provided by the coils 170, 172 and the coils 178, 180.

In some implementations, rather than the pulsing profiles 140 being previously stored in the console storage 118, the tip memory 174 may store data indicating pulsing profiles 140 specific to the tip 20. For instance, if the tip 20 is designed for ablating soft tissue, the tip memory 174 may store one or more soft tissue pulsing profiles 144 specific to the tip 20. Alternatively, if the tip 20 is designed for ablating hard tissue, then the tip memory 174 may store one or more hard tissue pulsing profiles 146 specific to the tip 20. In either case, responsive to the ultrasonic instrument 18 being coupled to the control console 16, the ultrasonic controller 112 may be configured to read the pulsing profiles 140 from the tip memory 174 and store the same in the console storage 118 and/or memory 122 for selection by the user.

The ultrasonic controller 112 may also be coupled and configured to drive the display 74 of the control console 16. Specifically, the ultrasonic controller 112 may be configured to generate information and user interface (UI) components for presentation on the display 74. Such information depicted on display 74 may include information identifying the handpiece 24 and the tip 20, and information describing the operating state of the ultrasonic tool system 12. When the display 74 is a touch screen display, the ultrasonic controller 112 may also be configured to cause the display 74 to depict images of buttons and other practitioner-selectable components. By interacting with the buttons and other practitioner-selectable components, the practitioner may set desired operating parameters for the ultrasonic tool system 12, such as those described herein.

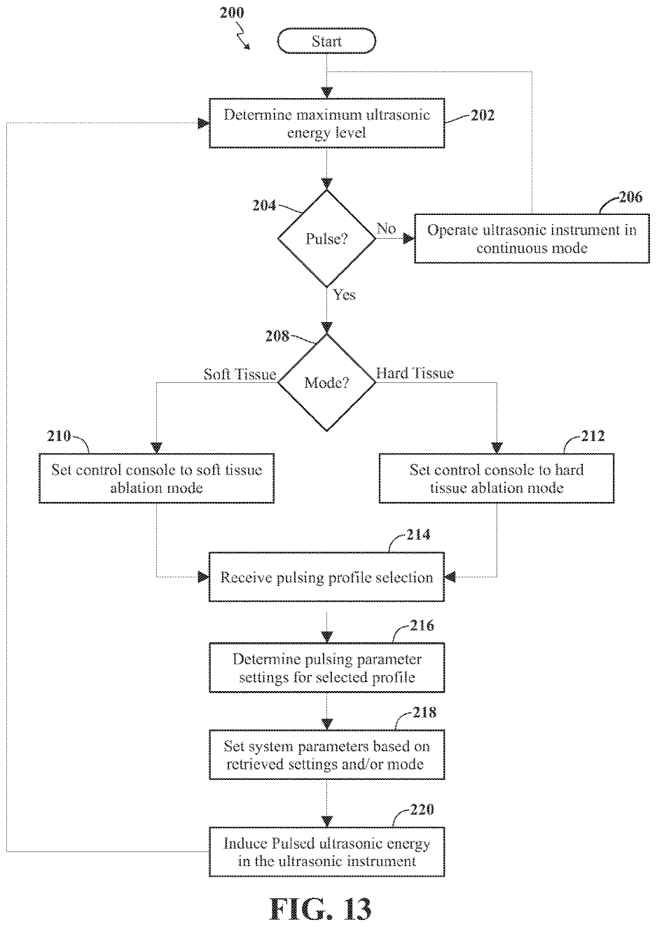

FIG. 13 illustrates a process 200 for controlling vibrations of the ultrasonic instrument 18 according to a selected pulsing profile 140. The control console 16, or more particularly the ultrasonic controller 112, may be configured to implement the process 200, such as upon execution of software 123 embodied by computer-executable instructions residing in the console storage 118.

In block 202, a maximum ultrasonic energy level for the induced ultrasonic energy may be determined. The maximum ultrasonic energy level may define a maximum mechanical current i_{M} for the ultrasonic instrument 18, and correspondingly, may define a maximum amplitude and velocity for the vibrations of the tip 20. The maximum ultrasonic energy level for the ultrasonic instrument 18 may be a based on the maximum ultrasonic energy level in which the ultrasonic instrument 18 is rated to accommodate, also referred to herein as the maximum capable ultrasonic energy level for the ultrasonic instrument 18 or a global ultrasonic energy limit, which may likewise be defined by a mechanical current i_{M}. The control console 16 may be configured to read the maximum capable ultrasonic energy level for the ultrasonic instrument 18 from the ultrasonic instrument 18, or more particularly, from the HP memory 168 and/or tip memory 174, responsive to the ultrasonic instrument 18 being connected to the control console 16.

The maximum ultrasonic energy level determined in block 202 may also be based on a power setting for the ultrasonic instrument 18 input by the practitioner. For instance, the practitioner may interact with the display 74 or remote control 80 of the ultrasonic tool system 12 to input a power setting for the ultrasonic instrument 18, which may indicate a percentage of the maximum capable ultrasonic energy level in which to limit driving the ultrasonic instrument 18. Responsive to receiving the percentage, the ultrasonic controller 112 may be configured to determine the maximum ultrasonic energy level for the ultrasonic instrument 18 by applying the percentage to the maximum capable ultrasonic energy level. In some instances, the ultrasonic controller 112 may further base the maximum ultrasonic energy level based on the depression angle of the foot pedal 76. Specifically, the depression angle of the foot pedal 76 may indicate to the ultrasonic controller 112 a percentage of the ultrasonic energy level corresponding to the user input power setting to use as the maximum ultrasonic energy level.

The practitioner may set the power setting and/or maximum ultrasonic energy level for the ultrasonic instrument 18 based on the personal preferences of the practitioner and the type of tissue targeted for ablation. As an example, for a given surgical procedure, a practitioner may target certain types of soft tissue for ablation via cavitation while avoiding ablation of other types of soft tissue. In this case, the practitioner may set the control console 16 to limit operation of the ultrasonic instrument 18 to an ultrasonic energy level that causes cavitation of the target tissue types while avoiding cavitation of other tissue types. For instance, relative to a brain procedure, the practitioner may desire to ablate one or more of dura mater, blood vessel walls, arachnoid matter, pia mater, white matter, or grey matter tissue while leaving other types of tissue intact. The ultrasonic instrument 18 may function to cavitate these types of tissue when the ultrasonic energy induced in the ultrasonic instrument 18 is approximately 27% of the maximum capable ultrasonic energy level for the ultrasonic instrument 18. Accordingly, the practitioner may set the control console 16 to limit operation of the ultrasonic instrument 18 to 30% of the maximum capable ultrasonic energy level for the ultrasonic instrument 18.

Combining a practitioner-selected power setting and/or maximum ultrasonic energy level with one of the pulsing profiles 140 may further help reduce potential trauma to tissue types desired to remain intact. For instance, continuing with the above example and referring to FIG. 7A, when the control console 16 is set to limit operation of the ultrasonic instrument 18 to 30% of the maximum capable ultrasonic energy level for the ultrasonic instrument 18, the maximum ultrasonic energy level induced by each soft tissue pulsing profile 144, as indicated by the 100% line, may correspond to 30% of the maximum capable ultrasonic energy level for the ultrasonic instrument 18. In this case, each soft tissue pulsing profile 144 may function to cavitate the target tissue when the ultrasonic energy induced by the soft tissue pulsing profile 144 is at or greater than the “cavitation threshold” near the maximum ultrasonic energy level of the soft tissue pulsing profile 144, which as described above may correspond to 27% of the maximum capable ultrasonic energy level for the ultrasonic instrument 18. The minimum ultrasonic energy level induced by each soft tissue pulsing profile 144 illustrated in FIG. 7A may be below the cavitation threshold. In this way, each soft tissue pulsing profile 144 may periodically induce ultrasonic energy sufficient to cause cavitation in the target tissue, and may therebetween induce reduced ultrasonic energy levels that function to reduce temperature and ablation of tissue types desired to remain intact.

Referring again to FIG. 13, in block 204, a determination may be made of whether pulsing mode is enabled for the ultrasonic instrument 18. Specifically, a practitioner may interact with the control console 16 to enable and disable pulsing mode, such as using the display 74 or remote control 80, and the ultrasonic controller 112 may be configured to make this determination based on the provided practitioner setting. Responsive to determining that pulsing mode is not enabled (“No” branch of block 204), in block 206, the control console 16 may be set to operate the ultrasonic instrument 18 in a continuous mode, such as according to the constant energy profile 148 described above. Specifically, upon actuation of the ultrasonic instrument 18, the ultrasonic controller 112 may be configured to generate and source an AC drive signal to the ultrasonic instrument that induces ultrasonic energy in the ultrasonic instrument 18 that is maintained at the determined maximum ultrasonic energy level for the ultrasonic instrument 18.

Additionally or alternatively, determining whether pulsing mode is enabled may include determining whether the tip 20 itself is pulsing enabled. In particular, some tips 20 releasably coupleable to the handpiece 24 may be configured for operation only in the continuous mode. Whether a tip 20 is pulsing enabled may be indicated as data specific to the tip 20 that is stored in the tip memory 174. Thus, responsive to the ultrasonic instrument 18 being coupled to the control console 16, the ultrasonic controller 112 may be configured to read the data from tip memory 174 and determine whether the tip 20 currently coupled to the handpiece 24 is pulsing enabled. In some instances, the data stored in the tip memory 174 may directly indicate whether the tip 20 is pulsing enabled. In other examples, the tip memory 174 may indicate the type of tip, such as via an identifier specific to the tip 20, and the ultrasonic controller 112 may be configured to query data stored in the console storage 118 corresponding to the tip type to determine whether the tip 20 is pulsing enabled. If not, then the ultrasonic controller 112 may be configured to disable this option from the practitioner, and set the control console 16 to operate the ultrasonic instrument 18 in the continuous mode as described above.

Alternatively, responsive to determining that the tip 20 is pulsing enabled, and/or that pulsing mode has been selected (“Yes” branch of block 204), in block 208, a determination may be made of whether the ultrasonic instrument 18 should be operated in the soft tissue ablation mode or hard tissue ablation mode. As previously described, each mode may be associated with a different set of pulsing profiles 140 specifically designed for the mode. A practitioner may set the control console 16 to either mode using a user interface associated with the control console 16, such as the display 74 or remote control 80, and the ultrasonic controller 112 may be configured to make this determination based on the provided practitioner setting.

Alternatively, the tip memory 174 distributed with the current tip 20 coupled to the handpiece 24 of the ultrasonic instrument 18 may include data indicating whether the tip 20 is configured for soft tissue ablation or hard tissue ablation. The data stored in the tip memory 174 may indicate whether the tip 20 is configured for soft tissue ablation or hard tissue ablation directly, or may indicate a type of the tip 20 that corresponds to data stored in the console storage 118 indicative of whether the tip type is for soft or hard tissue ablation. The control console 16 may be configured to read such data from the tip memory 174 when the ultrasonic instrument 18 is connected to the control console 16, as described above, to determine whether the ultrasonic instrument 18 should be operated in the soft tissue ablation mode or hard tissue ablation mode.

Responsive to determining that the ultrasonic instrument 18 is to be operated in the soft tissue ablation mode (“Soft Tissue” branch of block 208), in block 210, the control console 16 may be set to operate in soft tissue ablation mode. For example, the ultrasonic controller 112 may be configured to set a flag in the console storage 118 and/or memory 122 that corresponds to the control console 16 being set to the soft tissue ablation mode. Responsive to determining that the ultrasonic instrument 18 is to be operated in the hard tissue ablation mode (“Hard Tissue” branch of block 208), in block 212, the control console 16 may be set to operate in hard tissue ablation mode. For example, the ultrasonic controller 112 may be configured to set a flag in the console storage 118 and/or memory 122 that corresponds to the control console 16 being set to the hard tissue ablation mode.

In block 214, a practitioner-selection of one of the pulsing profiles 140 associated with the currently set ablation mode may be received. As previously described, the console storage 118 may store several soft tissue pulsing profiles 144 associated with the soft tissue ablation mode and several hard tissue pulsing profiles 146 associated with the hard tissue ablation mode. The varying soft tissue pulsing profiles 144 associated with the soft tissue ablation mode may be ordered, such as according to user-selectable pulse control levels assigned to the soft tissue pulsing profiles 144, so that each incremental soft tissue pulsing profile 144 provides increased tissue selectivity, temperature control, and/or tactile feedback, and the varying hard tissue pulsing profiles 146 associated with the hard tissue ablation mode may be ordered, such as according to user-selectable pulse control levels assigned to the hard tissue pulsing profiles 146, such that that each incremental hard tissue pulsing profile 146 provides increased tactile feedback regarding the tissue being contacted and how much force the practitioner is applying with the ultrasonic instrument 18, and/or increased stall potential. The practitioner may thus select one of the pulsing profiles 140 associated with the currently set ablation mode of the control console 16 by selecting the pulse control level for the pulsing profile 140, such as via the display 74 or remote control 80, based on the operating characteristics of the ultrasonic instrument 18 desired by the practitioner.

In block 216, responsive to receiving a practitioner-selection of one of the pulsing profiles 140 associated with the set ablation mode of the control console 16, the pulsing parameter settings associated with the selected pulsing profile 140 may be determined, such as by the ultrasonic controller 112 querying the console storage 118 based on the selected pulsing profile 140. Alternatively, the ultrasonic controller 112 may be configured to determine such pulsing parameter settings based on data read from the tip memory 174, which may store pulsing parameter settings for pulsing profiles 140 that are specific to the tip 20 and made selectable to the user upon the ultrasonic instrument 18 being connected to the control console 16.

In block 218, one or more system parameters may be set based on the retrieved pulsing parameter settings and/or the set ablation mode. For instance, the ultrasonic controller 112 may be configured to determine the minimum ultrasonic energy level for the ultrasonic instrument 18 based on the determined pulsing parameter settings, or more particularly the minimum energy factor indicated by the determined parameter settings.

As a further example, because fragmenting soft tissue typically does not require as much fragmentation power as fragmenting hard tissue such as bone, the ultrasonic controller 112 may be configured to reduce available power if the selected pulsing profile 140 corresponds to the soft tissue ablation mode, such as by setting a voltage limit for the AC drive signal to a lower value. Specifically, if the selected pulsing profile 140 corresponds to the soft tissue ablation mode, then the ultrasonic controller 112 may be configured to set the voltage limit for the AC drive signal to a relatively low value (e.g., 600 volts peak), and if the selected pulsing profile 140 corresponds to the hard tissue ablation mode, then the ultrasonic controller 112 may be configured to set the voltage limit for the AC drive signal to a relatively high value (e.g., 1200 volts peak). Alternatively, each pulsing profile 140 may include a pulsing parameter setting specific to the pulsing profile 140 that indicates a voltage limit to the use for the pulsing profile 140.

As a further example, the mechanical current i_{M} rate of change limit for the ultrasonic tool system 12 may regulate how quickly the control console 16 is enabled to induce a new target mechanical current i_{M} from a previously induced mechanical current mechanical current i_{M}. The mechanical current i_{M} rate of change limit utilized when operating in a continuous ultrasonic energy mode may not be fast enough for the pulsing mode, and accordingly, the ultrasonic controller 112 may be configured to set the mechanical current i_{M} rate of change limit to a higher value responsive to the ultrasonic tool system 12 being set to the pulsing mode and a pulsing profile 140 being selected. The mechanical current i_{M} rate of change limit set for a given pulsing profile 140 may depend on the pulse shape, pulsing frequency, and duty cycle of the pulsing profile 140. Accordingly, responsive to selection of a given pulsing profile 140, the ultrasonic controller 112 may be configured to set the mechanical current i_{M} rate of change limit based on these parameters, such as by using a formula or storing data associating varying values of these parameters with varying mechanical current i_{M} rate of change limits.

As another example, during operation of the ultrasonic instrument 18, the voltage of the power signal supplied by the power supply 165 may vary according to the voltage induced across the primary winding 124 so as to enable the amplifier 164 to generate the desired AC signal across the primary winding 124. To improve responsiveness of the system, the ultrasonic controller 112 may be configured to regulate the voltage of the signal supplied by the power supply 165 to the amplifier 164 based on voltages to be developed across the primary winding 124, as opposed or in addition to using a PID controller for the power supply 165 that waits for a feedback signal indicating a changed voltage. The regulation of the voltage of the signal supplied by the power supply 165 may be subject to a positive rate of change limit, and the ultrasonic controller 112 may be configured to implement a higher rate of change limit (e.g., two times) for this signal when operating in the pulsing mode rather than the continuous energy mode.

In some examples, such as when the control console 16 is set to operate in the hard tissue ablation mode, rather than the maximum ultrasonic energy level for the ultrasonic instrument 18 being set equal to the level set by the practitioner, such as via the user power setting and foot pedal 76, the ultrasonic controller 112 may be configured to determine the maximum ultrasonic energy level for the ultrasonic instrument 18 such that it is greater than the practitioner set level and the average ultrasonic energy level induced in the ultrasonic instrument 18 according to the selected pulsing profile 140 is substantially equal to the practitioner set level. This technique may result in higher resection rates and higher minimum ultrasonic energy levels for each pulsing profile 140, which may help prevent excessive stalling when treating hard tissue such as bone.

In block 220, an AC drive signal may be generated and sourced to the ultrasonic instrument 18 based on the determined maximum and minimum ultrasonic energy levels and the other pulsing parameter settings of the selected pulsing profile 140 as described above. Specifically, the AC drive signal may be set so as to induce ultrasonic energy in the ultrasonic instrument 18 having a plurality of ultrasonic energy pulses peaking at the maximum ultrasonic energy level and interspaced by ultrasonic energy at the minimum ultrasonic energy level according to the duty cycle and pulsing frequency of the selected pulsing profile 140.

As an example, responsive to selection of one of the soft tissue pulsing profiles 144 having a duty cycle of less than 100% (e.g., soft tissue pulsing profiles 144B to 144E of FIG. 7A, soft tissue pulsing profiles 144G to 144J of FIG. 8A), the control console 16 may be configured to generate the AC drive signal such that it induces ultrasonic energy in the ultrasonic instrument 18 that includes ultrasonic energy pulses peaking at the set maximum ultrasonic energy level and interspaced by significant periods at the minimum ultrasonic energy level (e.g., period greater than or equal to 2 milliseconds). The duration of each ultrasonic energy pulse relative to the duration of each cycle of the induced ultrasonic energy may correspond to the duty cycle associated with the selected soft tissue pulsing profile 144.

As another example, responsive to selection of a hard tissue pulsing profile 146 associated with a duty cycle of less than 100% (e.g., pulsing profiles 146B to 146E of FIGS. 7B and 146G to 146J of FIG. 8B), the control console 16 may be configured to generate the AC drive signal such that it induces ultrasonic energy in the ultrasonic instrument 18 that includes ultrasonic energy pulses peaking at the maximum ultrasonic energy level and interspaced by a momentary period (e.g., less than 1 millisecond) of ultrasonic energy at the minimum ultrasonic energy level. The peak of each ultrasonic energy pulse may include a significant period at the maximum ultrasonic energy level (e.g., period greater than or equal to 2 milliseconds). The duration of each pair of adjoining edges of adjacent ultrasonic energy pulses may correspond to the duty cycle associated with the selected hard tissue pulsing profile 146.

As a further example, responsive to selection of a pulsing profile 140 with an 100% duty cycle (e.g., pulsing profile 144A of FIG. 7A, 144F of FIG. 8A, 144K-144O of FIG. 9A, 146A of FIG. 7B, 146F of FIG. 8B, and 146K-146O of FIG. 9B), the control console 16 may be configured to generate the AC drive signal such that it induces ultrasonic energy in the ultrasonic instrument 18 that includes ultrasonic energy pulses interspaced by a momentary period at the minimum ultrasonic energy level (e.g., less than one millisecond), each of the ultrasonic energy pulses momentarily peaking (e.g., less than one millisecond) at the maximum ultrasonic energy level. In other words, the level of ultrasonic energy induced in the ultrasonic instrument 18 may be considered to be constantly fluctuating.

In some implementations, while the pulsed ultrasonic energy is being induced in the ultrasonic instrument 18, the control console 16 may display a toggle element that enables the practitioner to quickly switch between inducing pulsed ultrasonic energy according to the currently selected pulsing profile 140 and inducing ultrasonic energy in the ultrasonic instrument 18 according to the continuous ultrasonic energy mode (e.g., according to the constant energy profile 148) without, for example, having to stop the ultrasonic instrument 18 or traverse through each of the pulse control levels to disable pulsing. This feature may enable the practitioner to temporarily increase fragmentation power, such as if the practitioner encounters tissue difficult to ablate under the current settings, and then quickly return to the pulsed ultrasonic energy.

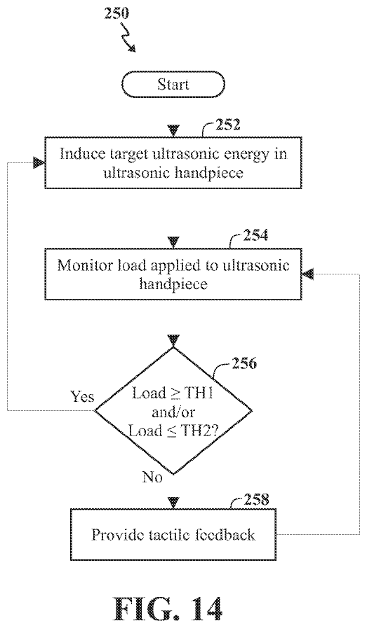

FIG. 14 illustrates a method 250 for providing tactile feedback to a practitioner using ultrasonic energy pulses induced in the ultrasonic instrument 18 to indicate whether the practitioner is providing an optimal amount of the pressure to the ultrasonic instrument 18. When the practitioner applies the vibrating tip 20 of the ultrasonic instrument 18 to tissue such as bone, the pressure that the practitioner applies to the ultrasonic instrument 18 may affect the efficacy of the tip 20 in resecting the tissue. If the practitioner applies too little pressure, then the tip 20 may not efficiently resect the tissue, and if the practitioner applies too much pressure, then the tip 20 may potentially ablate tissue desired to remain intact and/or may stall. The control console 16, or more particularly the ultrasonic controller 112, may be configured to implement the method 250 to provide tactile feedback to the practitioner that indicates whether the pressure being applied by the practitioner is too little, too great, or on target.

In block 252, target ultrasonic energy may be induced in the ultrasonic instrument 18, such as according to an ultrasonic energy profile selected by the practitioner. For instance, if one of the pulsing profiles 140 is selected to be induced in the ultrasonic instrument 18, then the control console 16 may be configured to induce pulsed ultrasonic energy in the ultrasonic instrument 18 according to the selected pulsing profile 140, with the ultrasonic energy pulses occurring at a default pulsing frequency (e.g., 50 Hz), as the target ultrasonic energy. In some examples, each pulsing profile 140 may define a default pulsing frequency specific to the pulsing profile 140. Alternatively, the control console 16 may be configured to use a same default pulsing frequency for each pulsing profile 140. Conversely, if pulsing mode is disabled by the practitioner, then the control console 16 may be configured to induce continuous ultrasonic energy in the ultrasonic instrument 18, such as according to the constant energy profile 148, as the target ultrasonic energy.

In block 254, a load applied to the ultrasonic instrument 18, or more particular to the mechanical components of the ultrasonic instrument 18, may be monitored. The magnitude of the load applied to the mechanical components of the ultrasonic instrument 18 may be a function of the physical properties of the tissue being contacted by the tip 20 and the force applied to the ultrasonic instrument 18 by the practitioner. As the practitioner applies increased pressure on the ultrasonic instrument 18, the load applied to the mechanical components may increase, and as the practitioner applies decreased pressure on the ultrasonic instrument 18, the load applied to the mechanical components may decrease.

The ultrasonic controller 112 may be configured to monitor the load applied to the ultrasonic instrument 18 by calculating a load measurement value indicating an extent of the applied load. In some examples, the load measurement value may be the mechanical impedance Z_{M} or the mechanical resistance R_{M} exhibited by the ultrasonic instrument 18 during operation, which may increase and decrease with the load applied to the mechanical components of the ultrasonic instrument 18. More specifically, referring back to FIGS. 6A and 6B, when the ultrasonic instrument 18 is operating at resonance (e.g., the base frequency of the AC drive signal, corresponding to the frequency of the base AC signal 154 of the signal generator 114, substantially equals the resonant frequency of the ultrasonic instrument 18), the inductive component L_{M} and the capacitive component C_{M} of the mechanical impedance Z_{M} of the ultrasonic instrument 18 may cancel each other out. Accordingly, when the ultrasonic instrument 18 is operating at resonance, the mechanical impedance Z_{M} of the ultrasonic instrument 18 may equal the mechanical resistance Ry of the ultrasonic instrument 18, which may be calculated using Ohm's law based on the mechanical current i_{M} and the voltage vₛ of the AC drive signal.

The ultrasonic controller 112 may thus be configured to determine a load measurement value for the ultrasonic instrument 18 by calculating the mechanical resistance R_{M} of the ultrasonic instrument 18 based on the mechanical current i_{M} of the ultrasonic instrument 18, such as determined using Equation (1) above, and the voltage vₛ of the AC drive signal, such as measured using the voltage measuring circuit 90, when the ultrasonic instrument 18 is operating at resonance. By Ohm's law, the mechanical impedance Z_{M} of the ultrasonic instrument 18 may equal the drive voltage vₛ divided by the mechanical current i_{M}. Because the mechanical impedance Z_{M} may equal the mechanical resistance R_{M} at resonance, the control console 16 may be configured to calculate the mechanical resistance R_{M} of the ultrasonic instrument 18 by dividing the drive voltage vₛ by the mechanical current i_{M} when the ultrasonic instrument 18 is operating at resonance.

As another example, the load measurement value may be the voltage vₛ of the AC drive signal, such as measured using the voltage measuring circuit 90. As previously described, the control console 16 may be configured to adjust the voltage vₛ of the AC drive signal so as to induce a target mechanical current i_{M} in the ultrasonic instrument 18. The mechanical current i_{M} of the ultrasonic instrument 18 may vary as a function of the mechanical impedance Z_{M} or the mechanical resistance R_{M} of the ultrasonic instrument 18 during operation. The voltage vₛ of the AC drive signal may thus vary as a function of the mechanical impedance Z_{M} or the mechanical resistance R_{M} exhibited by the ultrasonic instrument 18 during operation, and correspondingly may increase and decrease with the load applied to the mechanical components of the ultrasonic instrument 18.

Referring again to FIG. 14, in block 256, a determination may be made of whether an optimal load is being applied to the ultrasonic instrument 18, or more particularly to the mechanical components of the ultrasonic instrument 18. To this end, the control console 16 may be configured to determine whether the monitored applied load is within a target range defined by a predefined lower load threshold level (TH1) and a predefined upper load threshold level (TH2). More particularly, the control console 16 may be configured to determine whether the monitored applied load is greater than or equal to the lower threshold level (TH1) and/or less than or equal to the upper threshold level (TH2). As previously described, the applied load may be a function of the amount of pressure being applied by the practitioner to the ultrasonic instrument 18. The monitored applied load being less than the lower threshold level TH1 may indicate that the practitioner is providing less than optimal pressure for resecting tissue, and the monitored applied load being greater than the upper threshold level TH2 may indicate that the practitioner is providing greater than optimal pressure for resecting tissue. If the monitored applied load is within the target range, then the control console 16 may be configured to determine that an optimal load is being applied to the ultrasonic instrument 18 (“Yes” branch of block 256).

The control console 16 may be configured to determine if the monitored applied load is within the target range by being configured to determine if the load measurement value defining the monitored applied load is within the target range. The lower threshold level TH1 and upper threshold TH2 may thus be defined in the units of the load measurement value. For instance, if the load measurement value corresponds to the voltage vₛ of the AC drive signal, then the lower threshold level TH1 and upper threshold level TH2 may be defined by voltage thresholds in volts. Alternatively, if the load measurement value corresponds to the mechanical impedance Z_{M} or the mechanical resistance R_{M} of the ultrasonic instrument 18, then the lower threshold level TH1 and upper threshold level TH2 may be respectively defined by mechanical impedance thresholds or mechanical resistance thresholds in ohms. For example and without limitation, when the load measurement value corresponds to the mechanical resistance R_{M} of the ultrasonic instrument 18, the lower threshold level TH1 may be 2000 ohms, and the upper threshold level TH2 may be 5000 ohms.

In some implementations, the ultrasonic controller 112 may be configured to calibrate the load measurement value prior to determining whether the value is within the target range, such as based on the level (e.g. flow rate) of irrigating fluid being provided via the sleeve 42, which may be set by the practitioner and/or monitored by the control console 16, and/or on the type of tissue being contacted the operative end 22 of the tip 20, which may be detected as described in more detail below. More specifically, each of these items may affect the load on the mechanical components of the ultrasonic instrument 18, and increase or decrease the load measurement value accordingly. The console storage 118 may thus store data indicating values by which to offset (e.g., reduce) the load measurement value for different irrigating fluid settings/measurements and/or different detected tissue types being contacted to normalize the value. Additionally or alternatively, such data may be indicated in a memory device of the ultrasonic instrument 18 (e.g., the tip memory 174) and read by the ultrasonic controller 112 upon connection of the ultrasonic instrument 18 with the control console 16. Additionally or alternatively, the ultrasonic controller 112 may be configured to determine an offset normalization value by instructing the practitioner to vibrate the tip 20 in free air prior to a procedure and/or without irrigation, and measuring the corresponding load measurement value during such vibration to use as an offset value for normalizing the load measurement value later in the procedure.

Responsive to determining that the monitored applied is optimal (“Yes” branch of block 256), the method 250 may return to block 252 to continue inducing the target ultrasonic energy in the ultrasonic instrument 18, monitoring the applied load, and determining whether the monitored applied load is optimal. Responsive to determining that the monitored applied load is not optimal (e.g., the monitored applied load is less than the lower threshold level TH1 or greater than the upper threshold level TH2) (“No” branch of block 256), in block 258, tactile feedback indicating such condition may be provided to the practitioner.

For instance, responsive to determining that the monitored applied load is less than the lower threshold level TH1, the control console 16 may be configured to induce pulsed ultrasonic energy in the ultrasonic instrument 18 with a relatively high or low pulsing frequency, and responsive to do determining that the monitored applied load is greater than the upper threshold level TH2, the control console 16 may be configured to induce pulsed ultrasonic energy in the ultrasonic instrument 18 with the other of the relatively high or low pulsing frequency. For example and without limitation, responsive to determining that the monitored applied load is less than the lower threshold level TH1, the control console 16 may be configured to induce pulsed ultrasonic energy in the ultrasonic instrument 18 with a relatively high pulsing frequency of 60 Hz, and responsive to determining that the monitored applied load is greater than the upper threshold level TH2, the control console 16 may be configured to induce pulsed ultrasonic energy in the ultrasonic instrument 18 with a relatively low pulsing frequency of 10 Hz. In this example, when the practitioner is providing too little pressure (e.g., the monitored applied load is less than the lower threshold level TH1), the practitioner may feel relatively fast pulsing in the ultrasonic instrument 18, and when the practitioner is providing too much pressure (e.g., the monitored applied load is greater than the upper threshold level TH2), the practitioner may feel relatively slow pulsing in the ultrasonic instrument 18.

If the target ultrasonic energy induced in block 252 is pulsed ultrasonic energy corresponding to a pulsing profile 140, then in block 258, responsive to determining that the monitored applied load is less than the lower threshold level TH1, the control console 16 may be configured to induce pulsed ultrasonic energy in the ultrasonic instrument 18 according to the selected pulsing profile 140 but with a pulsing frequency greater than (or less than) the default pulsing frequency associated with the selected pulsing profile 140. In other words, the control console 16 may be configured to induce the relatively high (or relatively low) pulsing frequency. Similarly, responsive to determining that the monitored applied load is greater than the upper threshold level TH2, then the control console 16 may be configured to induce pulsed ultrasonic energy in the ultrasonic instrument 18 according to the selected pulsing profile 140 but with a pulsing frequency less than (or greater than) the default pulsing frequency associated with the selected pulsing profile 140. In other words, the control console 16 may be configured to induce the relatively low (or relatively high) pulsing frequency.

In alternative implementations, if the target ultrasonic energy induced in block 252 is pulsed ultrasonic energy corresponding to a pulsing profile 140, responsive to determining that the monitored applied load is less than the lower threshold level TH1 or greater than the upper threshold level TH2, the control console 16 may be configured to transition to inducing ultrasonic energy maintained at a substantially constant value, such as the maximum ultrasonic energy level determined of the ultrasonic instrument 18 (e.g., constant energy profile 148, FIG. 7A).

Conversely, if the target ultrasonic energy is substantially constant ultrasonic energy, then in block 258, responsive to determining that the monitored applied load is less than the lower threshold level TH1 or greater than the upper threshold level TH2, the control console 16 may be configured to transition to inducing pulsed ultrasonic energy in the ultrasonic instrument 18, such as according to one of the pulsing profiles 140, with a pulsing frequency equal to the default pulsing frequency associated with the pulsing profile 140, or equal to a relatively high or low pulsing frequency as described above.

More specifically, assuming the target ultrasonic energy is substantially constant ultrasonic energy, responsive to determining that the monitored applied load is less than the lower threshold level TH1 or greater than the upper threshold level TH2 in block 256, in block 258, the control console 16 may be configured to determine whether the currently connected tip 20 is configured for ablating hard or soft tissue, such as based on a user-provided setting or data read from the tip memory 174. The control console 16 may then be configured to induce one of the soft tissue pulsing profiles 144 responsive to determining that the tip 20 is configured for ablating soft tissue, and to induce one of the pulsing profiles 146 responsive to determining that the tip 20 is configured for cutting bone.

The pulsing frequency of the induced pulsed ultrasonic energy may be set to a default pulsing frequency associated with the pulsing profile 140 (e.g., 50 Hz), or may be set based on whether the monitored applied load was determined to be less than the lower threshold level TH1 or greater than the upper threshold level TH2 as described above. For instance, responsive to determining that the monitored applied load is less than the lower threshold level TH1, the control console 16 may be configured to induce a relatively high pulsing frequency (e.g., 60 Hz), and responsive to determining that the monitored applied load is greater than the upper threshold level TH2, the control console 16 may be configured to induce the relatively low pulsing frequency (e.g., 10 Hz), or vice versa.

Following block 258, the method 250 may return to block 254 to continue monitoring and comparing the applied load against the target range to determine if the applied load is optimal. Responsive to determining that the monitored applied load becomes optimal (e.g., becomes greater than or equal to the lower threshold level TH1 and less than or equal to the upper threshold level TH2) (“Yes” branch of block 256), the method 250 may return to block 252 in which the target ultrasonic energy may again be induced in the ultrasonic instrument 18 to indicate to the practitioner that an optimal amount of pressure is being applied. In other words, the control console 16 may induce ultrasonic energy in the ultrasonic instrument 18 that corresponds to the practitioner-selected ultrasonic energy profile and related settings.

For instance, if the practitioner has selected one of the pulsing profiles 140 to be induced in the ultrasonic instrument 18 as the target ultrasonic energy, then the control console 16 may be configured to induce the selected pulsing profile 140 with a pulsing frequency equal to the default pulsing frequency associated with the pulsing profile 140. Alternatively, if the practitioner has selected a constant energy profile 148 to be induced in the ultrasonic instrument 18, then the control console 16 may be configured to induce constant ultrasonic energy in the ultrasonic instrument 18. In either case, the practitioner may be able to feel the resumption of the target ultrasonic energy in the ultrasonic instrument 18, and associate such resumption with an indication that the practitioner is providing optimal pressure to the ultrasonic instrument 18.

In some examples, each pulsing profile 140 may be associated with a range of pulsing frequencies between the relatively high and relative low pulsing frequencies to induce depending on the magnitude of the monitored applied load within the target range. In other words, as the monitored applied load varies within the target range, the control console 16 may be configured to determine and induce a varying pulsing frequency in the ultrasonic instrument 18 as a function of the extent the monitored applied load varies.

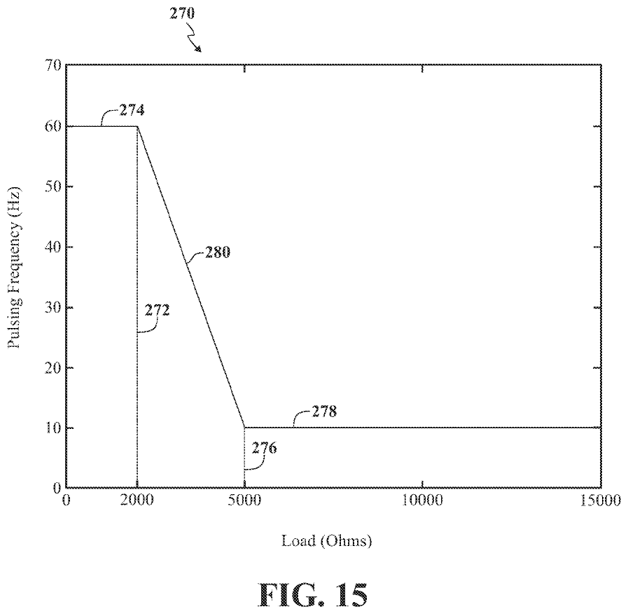

For instance, referring to FIG. 15, the console storage 118 or tip memory 174 may store data defining a graph 270 that associates various load measurement values between the lower threshold level TH1 and upper threshold level TH2 each with a unique pulsing frequency to induce in the ultrasonic instrument 18 when the load measurement value occurs. For instance, the data may indicate a lower threshold level TH1 272, a relatively high pulsing frequency 274 associated with the lower threshold level TH1 272, an upper threshold level TH2 276, a relatively low pulsing frequency 278 associated with the upper threshold level TH2 276, and a transition function 280. In the example illustrated in FIG. 15, the load measurement values are provided in ohms. In alternative examples, the load measurement values may be provided in other units, such as volts as described above.

The transition function 280 may extend from the relatively high pulsing frequency 274 associated with the lower threshold level TH1 272 to the relatively low pulsing frequency 278 associated with the upper threshold level TH2 276 over the load measurement values between lower threshold level TH1 272 and the upper threshold level TH2 276. In other words, the transition function 280 may associate each of the load measurement values greater than or equal to the lower threshold level TH1 272 and less than or equal to the upper threshold level TH2 276 with a unique pulsing frequency. The transition function 280 may be a decreasing function, such as linear function with a negative slope, so that the associated pulsing frequencies decrease as the load measurement values increase.

The control console 16 may be configured to induce pulsed ultrasonic energy in the ultrasonic instrument 18 with a varying pulsing frequency determined based on the above data. In particular, assuming the practitioner has selected one of the pulsing profiles 140 to be induced in the ultrasonic instrument 18, responsive to actuation of the ultrasonic instrument 18, the control console 16 may be configured to induce pulsed ultrasonic energy in the ultrasonic instrument 18 according to the selected pulsing profile 140 and with a pulsing frequency equal to a default pulsing frequency (e.g., 50 Hz) associated with the selected pulsing profile 140. Thereafter, the control console 16 may be configured to repeat cycles of monitoring the load applied to the mechanical components of the ultrasonic instrument 18, determining an updated pulsing frequency to induce in the ultrasonic instrument 18 based the monitored applied load and the graph 270, and generating pulsed ultrasonic energy in the ultrasonic instrument 18 according to the selected pulsing profile 140 and the updated pulsing frequency.

For instance, assuming the monitored applied load is defined by the mechanical resistance R_{M} of the ultrasonic instrument 18, the control console 16 may be configured to determine whether the calculated mechanical resistance Ry is less than or equal to the lower threshold level TH1 272, greater than or equal to the upper threshold level TH2 276, or between the lower threshold level TH1 272 and upper threshold level TH2 276. Responsive to determining that the mechanical resistance R_{M} is less than or equal to the lower threshold level TH1 272, the control console 16 may be configured to set the pulsing frequency of the pulsed ultrasonic energy induced in the ultrasonic instrument 18 to the relatively high pulsing frequency 274, and responsive to determining that the mechanical resistance R_{M} is greater than or equal to the upper threshold level TH2 276, the control console 16 may be configured to set the pulsing frequency of the pulsed ultrasonic energy induced in the ultrasonic instrument 18 to the relatively low pulsing frequency 278. Responsive to determining that the mechanical resistance Ry is between the lower threshold level TH1 272 and the upper threshold level TH2 276, the control console 16 may be configured to set the pulsing frequency of the pulsed ultrasonic energy induced in the ultrasonic instrument 18 to the pulsing frequency indicated by the transition function 280 as a function of the mechanical resistance R_{M}.

In this way, as the pressure applied by the practitioner to the ultrasonic instrument 18 deviates from a specified optimal pressure level, which may be indicated by the load measurement value varying from a predefined load measurement value (e.g., 3000 Ohms) between the lower threshold level TH1 272 and the upper threshold level TH2 276, the pulsing frequency of the pulsed ultrasonic energy induced in the ultrasonic instrument 18 may vary immediately and to an extent to which the applied pressure differs from the specified optimal pressure level. As a result, the practitioner may receive tactile feedback indicating the discrepancy from the specified optimal pressure level immediately, and may determine an amount of pressure to add to or remove from the ultrasonic instrument 18 to provide the specified optimal pressure level based on the level of tactile feedback. For example and without limitation, the predefined load measurement value between the lower threshold level TH1 272 and the upper threshold level TH2 276 corresponding to the specified optimal pressure level may be set to the average of the lower threshold level TH1 272 and upper threshold level TH2 276, or may be set to the load measurement value corresponding to the average or median of the range of pulsing frequencies defined by the transition function 280.

As described above, in the example illustrated in FIG. 15, the lower threshold level TH1 272 is associated with a relatively high pulsing frequency 274, the upper threshold level TH2 276 is associated with a relatively low pulsing frequency 278, and the transition function 280 decreases from the relatively high pulsing frequency 274 to the relatively low pulsing frequency 278 as the load measurement values increase. As another example, it is contemplated that the lower threshold level TH1 272 may be associated with the relatively low pulsing frequency 278, the upper threshold level TH2 276 may be associated with the relatively high pulsing frequency 274, and the transition function 280 may increase from the relatively low pulsing frequency 278 to the relatively high pulsing frequency 274 as the load measurement values increase.

In some implementations, rather than or in addition to defining target ultrasonic energy to be induced when the applied load is within the target range, the practitioner may be able to define target ultrasonic energy to be induced when the applied load is outside of the target range. In this way, responsive to determining that the applied load is outside the target range, indicating that the practitioner may not be applying optimal pressure to the ultrasonic instrument 18, the control console 16 may be configured to induce the outside-range target ultrasonic energy defined by the practitioner in the ultrasonic instrument 18. Conversely, if the monitored applied load is within the target range, then the practitioner may be applying optimal pressure to the ultrasonic instrument 18, and the control console 16 may be configured to provide tactile feedback indicating such condition, such as by inducing the inside-range target ultrasonic energy in the ultrasonic instrument 18 that has likewise been defined by the practitioner.

For instance, if the outside-range target ultrasonic energy induced in the ultrasonic instrument 18 is according to the constant energy profile 148 discussed above, then the control console 16 may be configured to induce pulsed ultrasonic energy in the ultrasonic instrument 18, such as according to one of the pulsing profiles 140 that has been selected by the practitioner, as the inside-range target ultrasonic energy. In this way, responsive to the monitored applied load corresponding to the target range, the ultrasonic instrument 18 may begin exhibiting vibrations corresponding to the practitioner-selected pulsing profile 140. If the outside-range target ultrasonic energy induced in the ultrasonic instrument 18 is according to one of the pulsing profiles 140, then the control console 16 may be configured to adjust the pulsing frequency of the pulsed ultrasonic energy, such as by increasing the pulsing frequency or decreasing the pulsing frequency relative to the pulsing frequency of the outside-range target ultrasonic energy induced in the ultrasonic instrument 18, when the applied load is within the target range.

In some instances, the pulsing frequency of the pulsed ultrasonic energy induced when the monitored applied load is within the target range may also be varied based on the distance between the load measurement value and the lower threshold level TH1 and the distance between the load measurement value and the upper threshold value TH2. For instance, responsive to the load measurement value moving nearer the lower threshold level TH1 or upper threshold level TH2 from a predefined load value therebetween, the control console 16 may be configured to induce a higher pulsing frequency. Similarly, responsive to the load measurement value moving from the lower threshold level TH1 or the upper threshold level TH2 towards the predefined load value, the control console 16 may be configured to induce a lower pulsing frequency. This configuration could also be reversed, such that movement of the load measurement value towards the lower threshold value TH1 or the upper threshold value TH2 from the predefined load value causes a lower pulsing frequency, and movement from the lower threshold level TH1 or the upper threshold level TH2 towards the predefined load value causes a higher pulsing frequency. The control console 16 may be configured to adjust the pulsing frequency in this manner according to a transition function that defines a specific pulsing frequency for each load measurement value between the lower threshold level TH1 and upper threshold level TH2, such as a bell curve function that takes as inputs the values between the lower threshold level TH1 and upper threshold level TH2 and peaks at the predefined load value. For example and without limitation, the predefined load value may be set to the average of the lower threshold level TH1 and upper threshold level TH2, or to the load measurement value corresponding to the average or median of the pulsing frequencies defined by the transition function.

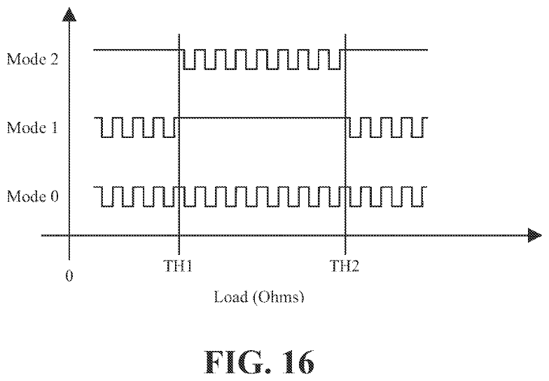

Referring to FIG. 16, in some implementations, the control console 16 may be configured to operate in varying pulsing activation modes selectable by the practitioner that automatically disable and enable pulsing based on the monitored load relative to the lower threshold level TH1 and the upper threshold level TH2. For instance, in pulsing activation mode 1, the control console 16 may be configured to induce pulsed ultrasonic energy, such as according to one of the pulsing profiles 140 selected by the practitioner, when the monitored load is outside the target range, and to induce substantially constant ultrasonic energy when the monitored load is inside the target range. Conversely, in pulsing activation mode 2, the control console 16 may be configured to induce pulsed ultrasonic energy, such as according to one of the pulsing profiles 140 selected by the practitioner, when the monitored load is inside the target range, and to induce substantially constant ultrasonic energy when the monitored load is outside the target range. Pulsing activation mode 0 may correspond to automatic pulsing activation being disabled.

The pulsing parameters described above, namely the lower threshold TH1 and the upper threshold level TH2, may be predetermined and stored in the console storage 118. In some instances, each pulsing profile 140 may define a lower threshold level TH1 and upper threshold level TH2 specific to the pulsing profile 140. Alternatively, the control console 16 may be configured to use the same lower threshold level TH1 and upper threshold level TH2 for each pulsing profile 140. In further implementations, rather than or in addition to a pulsing activation mode being selectable by the user, each pulsing profile 140 may define a specific pulsing activation mode (e.g., mode 1 or 2) available for the pulsing profile 140, and the practitioner may select between the defined pulsing activation mode or disabling pulsing activation mode when using that pulsing profile 140.

As previously described throughout this disclosure, different tips 20 may be releasably coupled to the handpiece 24 that have different operative characteristics. Because each tip 20 is typically distributed together with an irrigation sleeve 42 specific to the tip 20, to provide further optimization, optimized settings for the pulsing parameters described herein that are specific to each tip 20 removably coupleable to the handpiece 24 may be determined in advance and stored on the tip memory 174 of the irrigation sleeve 42 distributed with the tip 20. Thereafter, when the ultrasonic instrument 18 including the tip 20 is coupled to the control console 16, the control console 16 may be configured to read the data from the tip memory 174 that indicates the set pulsing parameters specific to the tip 20, and operate the ultrasonic instrument 18 based on the read data as described above.

For instance, the data stored on the tip memory 174 for a given tip 20 may indicate one or more pulsing profiles 140 specific to the tip 20 that may be selectable by the practitioner. To this end, the data stored on the tip memory 174 may indicate one or more pulsing parameter settings specific to the tip 20 including one or more of: one or more minimum energy factors specific to the tip 20, each of which may be associated with a different pulsing profile 140 specific to the tip 20; one or more duty cycles specific to the tip 20, each of which may be associated with a different pulsing profile 140 specific to the tip 20; one or more pulsing frequencies specific to the tip 20, each of which may be associated with a different pulsing profile 140 specific to the tip 20; one or more pulse shapes specific to the tip 20, each of which may be associated with a different pulsing profile 140 specific to the tip 20; and one or more voltage limits specific to the tip 20, each of which may be associated with a different pulsing profile 140 specific to the tip 20.

The data stored on the tip memory 174 for a given tip 20 may also indicate one or more other pulsing parameter settings specific to the tip 20, such as whether the tip 20 is pulsing enabled, whether the tip 20 is a hard tissue ablation tip or a soft tissue ablation tip, a lower threshold level TH1 for the tip 20, an upper threshold level TH2 for tip 20, and a pulse activation mode for the tip 20. Responsive to the ultrasonic instrument 18 including the tip 20 being coupled to the control console 16, the control console 16 may be configured to read this data from the tip memory 174 and utilize the indicated pulsing parameter settings as described above.

As a further example, the console storage 118 may also be configured to store the above pulsing parameters by tip type. In this case, responsive to the ultrasonic instrument 18 being coupled to the control console 16, the control console 16 may be configured to determine a type of the tip 20, such as from data read from the tip memory 174 indicating the tip type. The control console 16 may then be configured to query the console storage 118 for the pulsing parameter settings specific to the tip type, and utilize such pulsing parameter settings as described above.

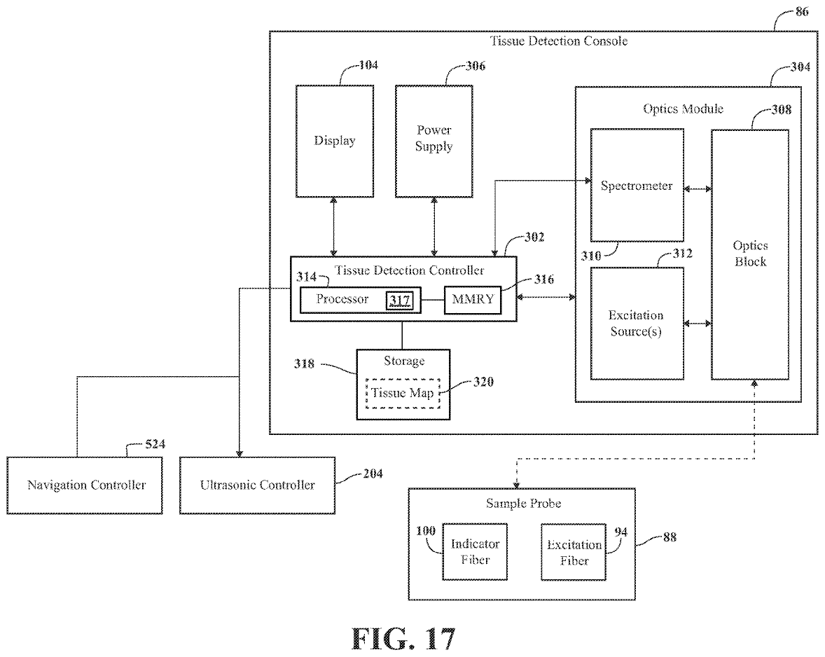

FIG. 17 illustrates components that may be integrated into the tissue detection control console 86 of the tissue detection system 13. The tissue detection control console 86 may include a controller 302, an optics module 304, and a power supply 306. The power supply 306 may be configured to supply power to other various components of the tissue detection control console 86 to enable operation of the same. The function of each other component will be discussed in greater detail below.

The optics module 304 may include an optics block 308, a spectrometer 310, and one or more excitation source(s) 312. Responsive to receiving a corresponding instruction from the tissue detection controller 302, the excitation source(s) 312 may illuminate the tissue being contacted by the operative end 22 of the tip 20 of the ultrasonic instrument 18 with excitation light via the excitation fiber 94. For example, the excitation source(s) 312 may be configured to emit blue light at about 405 nm or blue light in the range of 400 nm to 500 nm. The excitation source(s) 312 may also be configured to emit excitation light corresponding to other wavelengths, such as wavelengths associated with the rest of the visible light spectrum other than blue light (e.g., greater than 500 nm but less than 700 nm), wavelengths associated with the ultraviolet light spectrum (less than 400 nm), and/or wavelengths associated with the infrared light spectrum (greater than 700 nm). The excitation source(s) 312 may further include various types of light sources, including, but not limited to, a light emitting diode (LED), a pulsed laser, a continuous wave laser, a modulated laser, and/or a filtered white light source.

The optics block 308 may include a plurality of optical paths for directing light between the sample element 88 and components to the optics module 304. More particularly, the optics block 308 may include an optical pathway configured to direct light emitted from the excitation source(s) 312 down the excitation fiber 94 and indication fiber 100, and may also include an optical pathway configured to direct fluorescent light collected by the excitation fiber 94 to the spectrometer 310. The spectrometer 310 may be configured to convert the collected optical signals into electrical signals. More particularly, the spectrometer 310 may be configured to photoelectrically convert each wavelength of the collected optical signals into an electrical signal (also referred to herein as a spectral signal), which may then be provided to the tissue detection controller 302 for analysis.

The tissue detection controller 302 may be configured to implement the functions, features, and processes of the tissue detection control console 86 described herein. More specifically, similar to the ultrasonic controller 112, the tissue detection controller 302 may include a processor 314 and memory 316, and may include and/or be communicatively coupled to storage 318 of the tissue detection control console 86, with each being configured similarly to those described above in connection with the ultrasonic controller 112. The processor 314 may similarly operate under control of software programs 317 embodied by computer-executable instructions that, upon execution by the processor 314, causes the processor 314 to implement the functions, features, and processes of the tissue detection control console 86 described herein. In this manner, the tissue detection controller 302, or more particularly the processor 314, may be configured to periodically operate the optics module 304 to excite tissue being contacted by the operative end 22 of the tip 20 of the ultrasonic instrument 18, collect fluorescence light emitted from the tissue as a result of the tissue being excited, and convert the collected fluorescent light into spectral signals for analysis.

The tissue detection controller 302, or more particularly the processor 314, may also be configured evaluate the electrical signals to determine a characteristic of the tissue. To this end, the storage 318 may include tissue map data 320 that correlates characteristics of the spectral signals (e.g., frequency, intensity) with various tissue characteristics indicated by the spectral signals, such as the type of tissue (e.g., whether the contacted tissue is considered healthy or unhealthy tumorous tissue, and/or whether the tissue corresponds to a blood vessel), which may correspondingly indicate whether the contacted tissue is targeted for ablation or non-targeted. The tissue detection controller 302, or more particularly the processor 314, may thus be configured to access the tissue map data 320 to determine a characteristic of the tissue being contacted by the tip 20 of the ultrasonic instrument 18 based the florescent light collected by the sample element 88.

In some instances, the tissue map data 320 may define a plurality of tissue maps each associated with a different anatomy (e.g., brain, spine), with each tissue map correlating characteristics of the spectral signals with tissue characteristics in the context of the associated anatomy. In this case, the tissue detection controller 302, or more particularly the processor 314, may be configured to receive an indication of a given patient anatomy involved in a surgical procedure, such as via user input, and to query the tissue map associated with the indicated patient anatomy to determine the tissue characteristic.

The tissue detection system 13 may incorporate other features and functions, such as, and without limitation, those described in Applicant's International App. No. PCT/IB2022/052294, filed Mar. 14, 2022 and published as International Pub. No. WO 2022/190076 A1, the contents of which are hereby incorporated by reference herein in their entirety.

As illustrated in FIGS. 4 and 17, the ultrasonic controller 112 and tissue detection controller 302 may be communicatively coupled to each other. In this way, at least one of the controllers 112, 302 may be configured to communicate its determined tissue characteristic to the other controller 112, 302, such as to facilitate display and comparison of the tissue characteristics determined by the respective controllers 112, 302. These features are described in more detail in reference to the following methods.

As previously mentioned, and referring again to FIG. 1, the surgical system 10 may also include a navigation system 14 and imaging system 15. As will be appreciated from the subsequent description, the surgical system 10 may be configured to, among other things, allow the surgeon to visualize, approach, and treat or otherwise manipulate anatomy of a patient P at a target site TS with a high level of control. To this end, imaging data of the target site TS may be acquired via the imaging system 15, and can be used to assist the surgeon in visualizing the patient's P anatomy at or otherwise adjacent to the target site TS. The imaging data may also be utilized by the navigation system 14 to facilitate navigation of the ultrasonic instrument 18 relative to the target site TS, and to further enhance the tissue detection features described herein.

In the illustrative example of FIG. 1, a minimally invasive spinal surgical procedure, such as a posterior interbody spinal fusion, is being performed is being performed on a patient P. It will be appreciated that this example is intended to be illustrative, and that other types of surgical procedures are contemplated. In this exemplary surgical procedure, the ultrasonic instrument 18 may be employed to ablate vertebral disk tissue and cartilage end plate tissue. In other examples, the navigation system 14 may be configured to track a position and/or orientation of the ultrasonic instrument 18 as it operates to cut through bone, such as the skull, or as it operates to ablate tumorous tissue, such as in the brain. As the ultrasonic instrument 18 contacts tissues of varying characteristics, such as non-targeted (e.g., healthy) tissue and/or targeted (e.g., tumorous) tissue, the surgical system 10 may be configured to correlate the tissue characteristic(s) determined by ultrasonic tool system 12 and/or tissue detection system 13 with the tracked position and/or orientation of the ultrasonic instrument 18 to provide enhanced guidance to the practitioner and an additional data point for verifying the accuracy of the tissue detection.

As noted above, the imaging system 15 may be used to obtain imaging data of the patient P, which may be a human or animal patient. As shown in the representative configuration illustrated in FIG. 1, the imaging system 15 may be realized as an x-ray computed tomography (CT) imaging device. The patient P may be positioned within a central bore 506 of the imaging system 15, and an x-ray source and detector may be rotated around the central bore 506 to obtain raw x-ray imaging data of the patient P. The imaging data may be processed using an imaging controller 508, or another suitable controller, in order to construct three-dimensional imaging data, two-dimensional imaging data, and the like, which may be transmitted to or otherwise utilized by the navigation system 14.

The imaging data may be obtained preoperatively (e.g., prior to performing a surgical procedure) and/or intraoperatively (e.g., during a surgical procedure) by positioning the patient P within the central bore 506 of the imaging system 15. In order to obtain the imaging data, a portion of the imaging system 15 may be moved relative to a patient support 510 (e.g., a surgical table) on which the patient P is disposed while the patient P remains stationary. Here, the patient support 510 may be secured to the imaging system 15, such as via a column 512 mounted to a base 514 of the imaging system 15. A portion of the imaging system 15 (e.g., an O-shaped imaging gantry 516) which includes at least one imaging component may be supported by an articulable support 518 that can translate along the length of the base 514 on rails 520 to perform an imaging scan of the patient P, and may translate away from the patient P to an out-of-the-way position for performing a surgical procedure on the patient P.

An example imaging system 15 that may be used in various configurations is the AIRO® intra-operative CT system manufactured by Mobius Imaging, LLC. Examples of x-ray CT imaging devices that may be used according to various configurations of the present disclosure are described in U.S. Pat. No. 10,151,810, entitled “Pivoting Multi-directional X-ray Imaging System with a Pair of Diametrically Opposite Vertical Support Columns Tandemly Movable Along a Stationary Base Support”; U.S. Pat. No. 9,962,132, entitled “Multi-directional X-ray Imaging System with Single Support Column”; U.S. Pat. No. 9,801,592, entitled “Caster System for Mobile Apparatus”; U.S. Pat. No. 9,111,379, entitled “Method and System for X-ray CT Imaging”; U.S. Pat. No. 8,118,488, entitled “Mobile Medical Imaging System and Methods”; and U.S. Patent Application Publication No. 2014/0275953, entitled “Mobile X-ray Imaging System,” the disclosure of each of which is hereby incorporated by reference herein in its entirety.

While the illustrated imaging system 15 is realized as an x-ray CT imaging device, in other configurations, the imaging system 15 may include one or more of an x-ray fluoroscopic imaging device, a magnetic resonance (MR) imaging device, a positron emission tomography (PET) imaging device, a single-photon emission computed tomography (SPECT), or an ultrasound imaging device. Other configurations are contemplated. In some configurations, the imaging system 15 may be a mobile CT device that is not attached to the patient support 510 and may be wheeled or otherwise moved over the patient P and the patient support 510 to perform a scan. Examples of mobile CT devices include the BodyTom® CT scanner from Samsung Electronics Co., Ltd. and the O-arm® surgical imaging system from Medtronic, plc. The imaging system 15 may also be a C-arm x-ray fluoroscopy device. In other configurations, the imaging system 15 may be a fixed-bore imaging device, and the patient P may be moved into the bore of the device, either on a patient support 510 or on a separate patient table that is configured to slide in and out of the central bore 506. Further, although the imaging system 15 shown in FIG. 1 is located close to the patient P within the operating room, the imaging system 15 may be located remotely, such as in another room or building (e.g., in a hospital radiology department).

The surgical system 10 may employ the navigation system 14 to, among other things, track movement of various objects, such as the ultrasonic instrument 18 and parts of the patient's P anatomy (e.g., tissue in or adjacent the target site TS), as well as portions of the imaging system 15 in some configurations. To this end, the navigation system 14 may include a localizer 522 and a navigation controller 524 coupled to a localizer 522. The localizer 522 and the navigation controller 524 may be configured to cooperate to track the positions and/or orientations of trackers 526 disposed in the surgical workspace relative to the objects of interest.

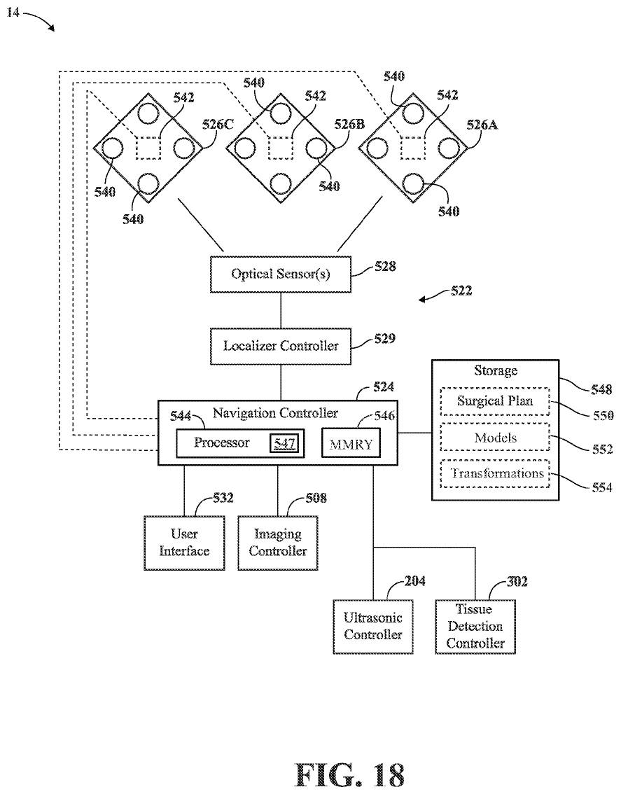

As shown in the illustrated example, the localizer 522 may be an optical localizer including a localizer camera unit 527. The localizer camera unit 527 may include an outer casing that houses one or more optical sensors 528 and a localizer controller 529 (FIG. 18). Each optical sensor 528 may be a separate charge-coupled device (CCD), and may be configured to detect light signals at a particular wavelength or in a particular frequency band, such as non-visible light (e.g., infrared). For example and without limitation, the optical sensor(s) 528 may consist of three one-dimensional CCDs, or may consist of two two-dimensional CCDs. In alternative examples, the optical sensor(s) 528 may be in the form of CMOS or other suitable sensors.

The localizer controller 529 (FIG. 18) may be configured to operate the optical sensor(s) 528, such as at the direction of the navigation controller 524, and to generate localizer data based on optical-based signals received from the optical sensor(s) 528. The localizer data may be indicative of the pixel positions of light signals detected by the optical sensor(s) 528 from the trackers 526, and correspondingly, may indicate the positions and/or orientations of the trackers 526 in a known coordinate system, such as a localizer coordinate system LCLZ specific to the localizer 522. The localizer controller 529 may be coupled and configured to communicate the localizer data to the navigation controller 524 for object tracking, described in more detail below. Alternatively, the optical sensors(s) 528 and navigation controller 524 may communicate directly.

The localizer 522 may be mounted to an adjustable arm to position the optical sensor(s) 528 with a field of view of the trackers 526 that, ideally, is free from obstructions. The localizer 522 may be adjustable in at least one degree of freedom by rotating about a rotational joint, and may be adjustable about two or more degrees of freedom.

Each of the trackers 526 may be affixed to an object of interest in the surgical workspace. In general, the object to which each tracker 526 is affixed may be rigid and inflexible so that during the surgical procedure, the object cannot or is unlikely to move or deform relative to the tracker 526. In other words, the spatial relationship between each tracker 526 and the object to which the tracker 526 is affixed may remain fixed during the surgical procedure, notwithstanding movement of the object during the surgical procedure. In this way, responsive to determining the position and/or orientation of a tracker 526 in the surgical workspace in a known coordinate system, the navigation controller 524 may infer the position of the object to which the tracker 526 is affixed in the known coordinate system based on the determined position and/or orientation of the tracker 526 and the fixed spatial relationship between the tracker 526 and object.

The objects tracked by the navigation system 14, and to which the trackers 526 may thus be affixed, may include patient anatomical structures of interest and instruments such as the ultrasonic instrument 18 and the imaging system 15. The tracked anatomical structures may include hard tissues such as bone and soft tissues such as skin. The tracked surgical instruments may include retractors, cutting tools, and waste management devices used during the surgical procedure.

For the example procedure illustrated in FIG. 1, the trackers 526 may include a tool tracker 526A for tracking a position and/or orientation of the ultrasonic instrument 18 in the known coordinate system, a patient tracker 526B for tracking a position and/or orientation of the patient and target site TS in the known coordinate system, and an imaging system tracker 526C for tracking a position and/or orientation of at least a portion of the imaging system 15 in the known coordinate system. Additional trackers 526, such as additional patient trackers and additional trackers for other medical and/or surgical tools, are also contemplated.

The tool tracker 526A and the imaging system tracker 526C are each depicted generically and are mounted to the ultrasonic instrument 18 and the gantry 516 of the imaging system 15, respectively. Patient trackers 526B may be firmly affixed to different portions of the patient's P anatomy (e.g., to opposing lateral sides of the ilium) via mount assemblies that are configured to releasably engage tissue (e.g., skin, bone). It will be appreciated that trackers 526 may be firmly affixed to different types of tracked objects (e.g., discrete bones, tools, pointers, and the like) in a number of different ways.

Prior to the start of a surgical procedure, the navigation controller 524 may receive and store data indicative of virtual models of the anatomy of the patient P that is of interest, such as based on pre-operative images of the anatomy of interest, which may be generated by the imaging system 15. Pre-operative images may be based on MRI scans, radiological scans, or computed tomography (CT) scans of the patient's anatomy, and may be used to develop virtual models of the anatomical structures of interest stored by the surgical navigation system 14. Each virtual model may include a three-dimensional model (e.g., point cloud, mesh, CAD) of at least portion of the anatomical structure, and/or may include a three-dimensional model or other indicator of at least a portion of the anatomical structure that forms at least part of a target volume of patient tissue to be treated during the surgical procedure. In addition or alternatively to taking pre-operative images, plans for treatment and virtual models for anatomical structures of interest can be developed in the operating room from kinematic studies, bone tracing, and other methods. Further prior to the surgical procedure, the navigation controller 524 may receive and store virtual models for other tracked objects of interest to the surgical procedure, such as virtual models of the surgical instruments (e.g., the ultrasonic instrument 18) and the trackers 526 being used in the surgical procedure.

Each virtual model for an object received and stored by the navigation controller 524 may define a three-dimensional coordinate system specific to the object, and may indicate coordinates in the three-dimensional coordinate system corresponding to the relative positions of features of the object. For instance, a virtual model for a given tracker 526 may indicate coordinates in a three-dimensional coordinate system specific to the tracker 526 that correspond to the relative positions of markers of the tracker 526, described in more detail below. As a further example, a virtual model for a given surgical instrument may indicate coordinates in a three-dimensional coordinate system specific to the surgical instrument that correspond to the relative positions of features of the housing of the surgical instrument.

The navigation controller 524 may also receive and store data defining the fixed spatial relationships between the trackers 526 and the objects to which the trackers 526 are affixed, and a surgical plan. The spatial relationships may define a position and/or orientation of each tracker 526, or more particularly a position and/or orientation of the markers of each tracker 526, relative to the object to which the tracker 526 is affixed, such as by reference to the virtual models of the object and tracker 526. For instance, the spatial relationship for a given tracker 526 may indicate where the coordinate system specific to the object affixed to the tracker 526 is positioned within the coordinate system specific to the tracker 526, and/or vice versa. The surgical plan may identify the patient anatomical structures involved in the surgical procedure and the target volume of patient tissue to be treated in the surgical procedure, may identify the instruments being used in the surgical procedure, and may identify planned trajectories of the instruments and planned movements of patient anatomical structures during the surgical procedure.

The spatial relationship between the patient tracker 526B and the anatomy of the patient P to which it is attached can be determined by known registration techniques, such as point-based registration in which a calibration tool including a calibration tracker is used to touch off points across the anatomy being registered. By monitoring the position and/or orientation of the calibration tracker relative to that of the patient tracker 526B in a known coordinate system (e.g., the localizer coordinate system LCLZ) as the calibration tool is touched off the points of the anatomy, the navigation controller 524 may be configured to determine a position and/or orientation of the anatomy relative to the patient tracker 526B based on a known spatial relationship between the touch point of the calibration tool and the calibration tracker. In addition or alternatively, the navigation controller 524 may be configured to determine the spatial relationship of the patient tracker 526B relative to the anatomy of the patient P based on imaging data generated by the imaging system 15, which may depict a position and/or orientation of the patient tracker 526B and of the patient anatomy in a same coordinate system, such as that of the imaging system 15. The navigation controller 524 may thus be configured to determine the spatial relationship of the patient tracker 526B relative to the anatomy of the patient P based on this information of the imaging data.

During the surgical procedure, the optical sensor(s) 528 of the localizer 522 may detect light signals, such as non-visible light signals (e.g., infrared or ultraviolet), emitted from the trackers 526. Responsive to detecting these light signals, the optical sensor(s) 528 may generate and communicate optical-based signals to the localizer controller 529, which may be configured to generate localization data from the optical-based signals that indicates the pixel positions and correspondingly the directions from which the detected light signals were received by the optical sensor(s) 528. The localizer controller 529 may be configured to communicate the localization data to the navigation controller 524, which may then be configured to determine tracker position and/or orientation data based on the localization data. The navigation controller 524 may then be configured to determine object position and/or orientation data indicative of positions and/or orientations of the objects to which the trackers 526 are affixed based on the determined tracker positions and/or orientations and the previously stored fixed spatial relationships between the trackers 526 and objects.

As shown in the example illustrated in FIG. 1, the navigation controller 524 and the localizer 522 may be supported on a mobile cart 530 which is movable relative to the base 514 of the imaging system 15. The mobile cart 530 may also support a user interface, generally indicated at 532, to facilitate operation of the navigation system 14 by displaying information to, and/or by receiving information from, the surgeon or another user. The user interface 532 may be disposed in communication with the navigation controller 524, and may include one or more output devices (e.g., monitors, indicators, display screens, speakers, and the like) to provide information to the surgeon. For instance, as shown in the illustrated example, the output devices of the user interface 532 may include, without limitation, a display 534 adapted to be situated outside of a sterile field including the surgical workspace, and may include a display 536 adapted to be situated inside the sterile field. The displays 534, 536 may be adjustably mounted to the mobile cart 530, and may each incorporate touch screen technology for receiving user input from the surgeon or other user. Other input devices of the user interface 532 for receiving user input may include, without limitation, a keyboard, mouse, and/or microphone that enables user-input through voice-recognition technology.

Responsive to determining the object position and/or orientation data indicative of the positions and/or orientations of the tracked objects in a known coordinate system, the navigation controller 524 may be configured to display virtual representations of the relative positions and/or orientations of the tracked objects to the surgeon or other users of the surgical system 10, such as with images and/or graphical representations of the anatomy of the patient P and the ultrasonic instrument 18 presented on the displays 534, 536. The navigation controller 524 may also be configured to utilize the user interface 532 to display instructions or request information from the surgeon or other users of the surgical system 10.

Referring now to FIG. 18, the trackers 526 affixed to objects in the surgical workspace may each include a known arrangement of markers 540 for emitting light signals detectable by the optical sensor(s) 528. In one example, the trackers 526 may be powered, and may thus include an arrangement of powered markers 540 each configured to emit light signals responsive to receiving an electrical current therethrough. As an example, the powered markers 540 may be realized as light emitting diodes (LEDs) that transmit light, such as non-visible light (e.g., infrared or ultraviolet), detectable by the optical sensor(s) 528. These trackers 526 may be powered by an internal battery, or may have leads to receive power through the navigation controller 524.

Each powered tracker 526 may also include a tracker controller 542 connected to the powered markers 540 of the tracker 526, and may be configured to control the rate and order in which the powered markers 540 fire, such as at the direction of the navigation controller 524. The navigation controller 524 may thus be in data communication with the tracker controllers 542, such as wirelessly or through a wired connection, to cause firing of the powered markers 540 of each tracker 526. The tracker controllers 542 may cause the markers 540 of each powered tracker 526 to fire at different rates and/or times to enable the navigation controller 524 to identify which marker 540 is firing at a given moment. In this way, the navigation controller 524 may associate a given detected light signal with the marker 540 that was firing when the light signal was detected. Alternatively, the tracker controllers 542 may cause the markers 540 to fire continuously and/or at a same time. In this case, each tracker 526 may include a unique, known pattern of markers 540, and the navigation controller 524 may be configured to implement a marker assignment algorithm, which may include matching light signals detected by different optical sensors 528 that correspond to a same marker 540, triangulating the position of the marker 540 corresponding to each set of matched light signals, and comparing the triangulated positions to the known tracker marker patterns to determine which light signals correspond to which markers 540 of the trackers 526.

Rather than being powered, one or more of the trackers 526 affixed to objects in the surgical workspace may be non-powered and thus include passive markers 540, such as reflectors that reflect light emitted from a light source near the surgical workspace (e.g., of the localizer 522). The reflected light may then be received and detected by the optical sensor(s) 528, and assigned to the markers 540 as described above.

The navigation system 14, or more particularly the localizer 522, may have other suitable components or structure not specifically recited herein. Furthermore, any of the techniques, methods, and/or components described herein with respect to the camera-based navigation system 14 shown throughout the drawings may be implemented or provided for any of the other configurations of the navigation system 14 described herein. For example, the navigation system 14 may also be based on one or more of inertial tracking, ultrasonic tracking, image-based optical tracking (e.g., with markers defined by patterns, shapes, edges, and the like that can be monitored with a camera), or any combination thereof.

The navigation controller 524 may be configured to implement the functions, features, and processes of the navigation system 14 described therein. More specifically, similar to the ultrasonic controller 112 and the tissue detection controller 302, the navigation controller 524 may include a processor 544 and memory 546, and may include and/or be communicatively coupled to storage 548, each being configured similarly to those described above in connection with the ultrasonic controller 112 and tissue detection controller 302. For example, the processor 544 may similarly operate under control of software programs 547 embodied by computer-executable instructions, such as a localization engine, transformer, a navigator, a segmentation tool, and a tissue tracker, each described in more detail below. The storage 548 may include data facilitating the functions, features, and processes of the navigation controller 524 described herein, such as surgical plan data 550, virtual model data 552, and transformation data 554.

The localization engine software upon execution may be configured to receive the localization data from the localizer controller 529, and to determine tracker position and/or orientation data indicative of the positions and/or orientations of the trackers 526 in a known coordinate system based on the localization data, such as by triangulating the positions of the markers 540 of each tracker 526 in the known coordinate system based on the localization data as described above. The transformer software upon execution may be configured to determine object position and/or orientation data indicative of the positions and/or orientations of the objects to which the trackers 526 are affixed based on the tracker position and/or orientation data. For example, the stored transformation data 554 may indicate the fixed spatial positions between the trackers 526 and the objects to which the trackers 526 are affixed, which may be retrieved by the transformer and applied to the tracker position and/or orientation data to determine the object position and/or orientation data. The navigator software may be configured to provide navigation guidance based on the determined object position and/or orientation data, or more particularly based on the tracked positions and/or orientations of the objects to which the trackers 526 are affixed in the known coordinate system. As an example, the navigator may be configured to access virtual models of the objects from the virtual model data 552, and to display and update virtual boundaries corresponding to the models on the user interface 532 to positions corresponding to the relative positions of the objects indicated by the object position and/or orientation data.

The segmentation tool software may be configured upon execution to generate one or more virtual boundary(s) in the known coordinate system and corresponding to the objects of interest, such as by applying a segmentation algorithm to imaging data received from the imaging system 15. The segmentation algorithm may be configured to employ various technologies in machine vision to determine the virtual boundary(s) associated various objects and/or tissue types within the image represented by the imaging data. In some examples, the segmentation algorithm may be configured to receive as inputs the medical image and the type of procedure to be performed on the target site TS as indicated in the surgical plan data 550, apply one or more of edge detection, clustering, and other segmentation algorithms calibrated based on the type of procedure to the medical image to identify boundaries between various objects and types of tissue within the image, and generate corresponding virtual boundary(s) within an image coordinate system specific to the imaging system 15. In addition or alternatively, a user may be able to interact with the user interface 532 of the navigation system 14 (or a user interface 531 of the imaging system 15) to manually define such virtual boundary(s) within the image coordinate system and/or to manipulate the virtual boundary(s) generated by the segmentation algorithm described above. The transformer may then be configured to determine positions and/or orientations of the virtual boundary(s) in the known coordinate system, such as the localizer coordinate system LCLZ, based on the tracker position and/or orientation data indicative of the position of the imaging system tracker 526C in the known coordinate system, and transformation data 574 indicative of a predetermined spatial relationship between the imaging system tracker 526C and the coordinate system specific to the imaging system 15.

The tissue tracker software may be configured upon execution to correlate the tissue characteristic(s) determined by the ultrasonic tool system 12 and/or the tissue detection system 13 with the tracked position and/or orientation of the ultrasonic instrument 18, or more particularly the position of the operative end 22 of the tip 20, relative to the other tracked objects when the tissue characteristic(s) are determined. The tissue tracker software may also be configured to display at least one indicator in the known coordinate system corresponding to the determined tissue characteristic(s) at the tracked position, and to verify that the determined tissue characteristics are consistent with the tissue being contacted by the ultrasonic instrument 18 that is indicated by the localization data and/or medical image. For example, the surgical plan data 550 may indicate the characteristics of the various tissues associated with the generated virtual boundary(s), which may be predetermined in advance, such as by the segmentation tool, and/or input by the surgeon or other user via the user interface 532. The tissue tracker software may thus be configured to determine, based on the tracked position and/or orientation of the ultrasonic instrument 18 relative to the virtual boundary(s), one or more characteristics of tissue being contacted by the operative end 22 of the tip 20 that is indicated by the surgical plan data 550, and to compare the determined tissue characteristic(s) to the characteristic(s) determined by the ultrasonic tool system 12 and/or tissue detection system 13 to verify consistency between the same.

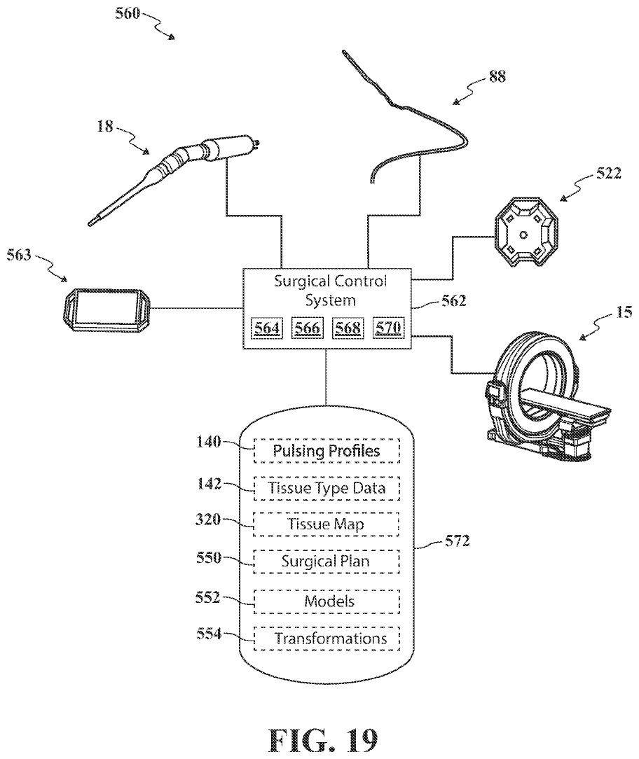

FIG. 19 illustrates a processing architecture 560 that may be implemented by the surgical system 10 of FIG. 1. As shown in the illustrated example, the processing architecture 560 may include a surgical control system 562, a user interface 563 communicatively coupled to the surgical control system 562, and one or more databases 572. The surgical control system 562 may also be operatively coupled to one or more of the ultrasonic instrument 18, the sample element 88, the localizer 522, and the imaging system 15.

The surgical control system 562 may generally be configured to regulate the ultrasonic energy induced in the ultrasonic instrument 18, such as to induce pulsed ultrasonic energy, based on the type of tissue being contacted by the operative end 22 of the ultrasonic tip 20, and/or may be configured to verify the tissue detection and/or navigation routines described herein. Specifically, the surgical control system 562 may include or be implemented by one or more of the ultrasonic controller 112 of the ultrasonic tool system 12, the navigation controller 524 of the surgical navigation system 14, the imaging controller 508 of the imaging system 15, and the tissue detection controller 302 of the tissue detection system 13. For instance, one or more of the above controllers may each be configured to implement one or more of the functions of the surgical control system 562 that are described herein, such as upon execution of corresponding software embodied by computer-executable instructions by the controller. In other words, the surgical control system 562 may be distributed across two or more of the above controllers, which may thus form a control system cooperating with other components of the surgical system 10 to implement the various functions, features, methods, and processes of the surgical system 10 described herein.

In some instances, the surgical control system 562 may include a software suite including a plurality of software programs or modules, each executing on at least one of the above controllers. For instance, the surgical control system 562 may include an ultrasonic module 564, which may be executed on at least the ultrasonic controller 112 and/or the navigation controller 524, and may be configured upon execution to regulate the ultrasonic energy induced in the ultrasonic instrument 18, such as based on a determined type of tissue being contacted by the operative end 22 of the ultrasonic tip 20. The surgical control system 562 may further include a navigation module 566, which may be executed on at least the navigation controller 524, and may be configured upon execution to gather localization data indicating the position and/or orientation of the operative end 22 of the ultrasonic tip 20, and generate tissue contact data that, based on a medical image of the patient, indicates a type of tissue which the operative end 22 of the ultrasonic tip 20 is contacting. The surgical control system 562 may further include an imaging module 568, which may be executed by at least the imaging controller 508, and may be configured upon execution to generate imaging data defining a medical image of a patient, or more particularly of a target site of the patient. The surgical control system 562 may further include a tissue detection module 570, which may be executed by at least the tissue detection controller 302, and may be configured upon execution to determine a type of tissue being contacted by the operative end 22 of the ultrasonic tip 20 as described in more detail below.

The one or more databases 572 of the processing architecture 560 may likewise be implemented by one or more of the console storage 118 of the ultrasonic tool system 12, the storage 318 of the tissue detection system 13, and the storage 548 of the surgical navigation system 14. For instance, one or more of the above storages may each store at least a portion of the one or more databases 572. In other words, the one or more databases 572 may be distributed across two or more of the above storages. The one or more databases 572 may store data used by the surgical control system 562, or more particularly by the modules of the surgical control system 562, to facilitate the functions, features, processes, and methods of the surgical control system 562 described herein. For instance, the one or more databases 572 may store one or more of the pulsing profiles 140 and tissue type data 142 described above in connection with the ultrasonic tool system 12, the tissue map data 320 described above in connection with the tissue detection system 13, and the surgical plan data 550, virtual model data 552, and transformation data 554 described above in connection with the navigation system 14.

The user interface 563 may facilitate user interaction with the surgical control system 562. More specifically, the user interface 563 may include one or more output components, such as a display, for presenting information to a user from the surgical control system 562, and may include one or more inputs, such as a touch screen, for receiving inputs from a user for the surgical control system 562. For instance, the user interface 563 may include one or more of the foot pedal 76, remote control 80, and display 74 of the ultrasonic tool system 12, the user interface 532 of the surgical navigation system 14, the user interface 531 of the imaging system 15, and the display 104 of the tissue detection system 13. In other words, the user interface 563 may be distributed across two or more of the above systems.

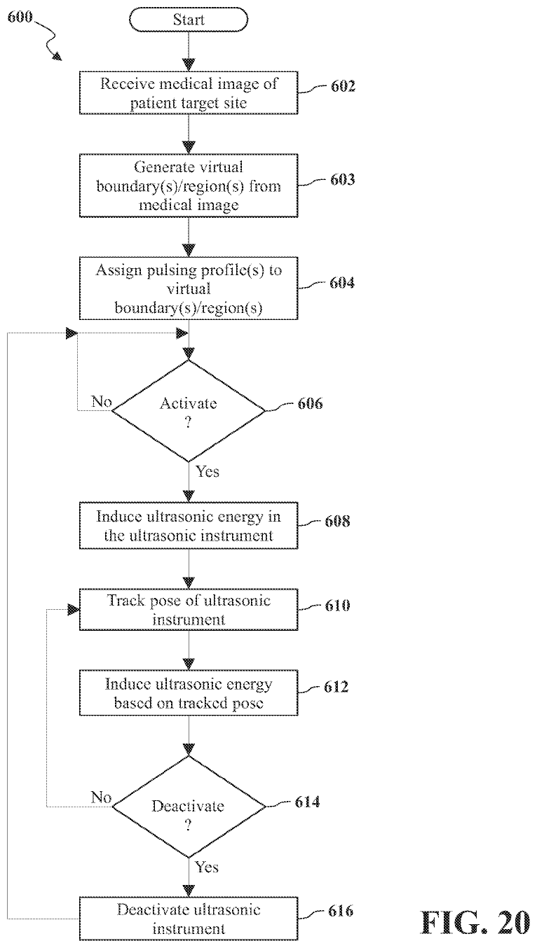

FIG. 20 illustrates a method 600 for operating the ultrasonic tool system 12, or more particularly the ultrasonic instrument 18, based on image data generated by the imaging system 15 and localization data generated by the localizer 522. The method 600 may be implemented by the surgical control system 562, or more particularity by the navigation controller 524 and/or the ultrasonic controller 112.

In block 602, a medical image of a target site of the patient may be received, such as by the surgical control system 562. For instance, the navigation controller 524 may receive image data from the imaging system 15 that defines an image of the patient including target site. The target site may generally include a region of tissue targeted for ablation (e.g., a tumorous tissue region), and may include a region of tissue not targeted for ablation (e.g., healthy tissue surrounding tumorous tissue), according to a surgical plan.

In block 603, based on the medical image, one or more virtual boundaries and/or virtual regions associated with the region of tissue targeted for ablation ablated may be generated, such as by the surgical control system 562, in a known coordinate system, such as the localizer coordinate system LCLZ. As described in more detail below, the surgical control system 562, or more particularly the navigation controller 524 as an example, may be configured to track the pose of the ultrasonic instrument 18 relative to the virtual boundary(s) and/or region(s) in the known coordinate system, and regulate the ultrasonic energy induced in the ultrasonic instrument 18 based thereon.

For instance, the surgical control system 562, or more particularly the navigation controller 524 as an example, may be configured to generate an outer edge virtual boundary corresponding to a boundary or edge of the tissue targeted for ablation, such that the virtual boundary is in between or separates the region of tissue targeted for ablation (also referred to herein as “target tissue”) and the region of tissue not targeted for ablation (also referred to herein as “non-target tissue”). In addition to the outer edge virtual boundary, the surgical control system 562 may be configured to generate one or more interior virtual boundaries within the target tissue region, such as at one or more threshold distances from the outer edge virtual boundary. The surgical control system 562 may also be configured to generate one or more virtual regions corresponding to the areas or volumes defined between and/or adjacent the virtual boundaries. As non-limiting examples, each virtual boundary and/or region may be realized by a line, plane, or surface mesh generated in a known coordinate system, such as the localizer coordinate system LCLZ.

In some implementations, the surgical control system 562, or more particularly the navigation controller 524 as an example, may be configured to generate the one or more virtual boundaries and/or regions by applying a segmentation algorithm to the received medical image, which may be configured to employ various technologies in machine vision to determine the virtual boundary(s) and/or region(s) of various tissue types within the image. In some examples, the segmentation algorithm may be configured to receive as inputs the medical image and the type of procedure to be performed on the target site as indicated in the surgical plan data 550, apply one or more o edge detection, clustering, and other segmentation algorithms calibrated based on the type of procedure to the medical image to identify boundaries and/or regions between various types of tissue within the image, and generate corresponding virtual boundaries and/or regions within an image coordinate system associated with the medical image (e.g., a coordinate system specific to the imaging system 15). The segmentation algorithms specific to various procedure types may be generated from artificial intelligence processes. In addition or alternatively, a user may be able to interact with the user interface 556, or more particularly the user interface 532 of the navigation system 14, coupled to the surgical system manger 562, to manually define virtual boundaries and/or regions within the image coordinate system, and/or to manipulate the virtual boundaries and/or regions generated by the segmentation algorithms described above.

The surgical control system 562, or more particularly the navigation controller 524 as an example, may then be configured to transform the virtual boundaries and/or regions defined in the image coordinate system associated with the image data to another known coordinate system, such as the localizer coordinate system LCLZ, so as to enable tracking the pose of the ultrasonic instrument 18 relative to the boundaries and/or regions. For instance, the navigation controller 524 may be configured to determine a pose of the imaging system tracker 526C within the localizer coordinate system LCLZ. Thereafter, based on the previously stored transformation data 554 indicating a known spatial relationship between the image coordinate system and a coordinate system of the imaging system tracker 526C, the navigation controller 524 may be configured to transform the virtual boundaries and/or regions from the image coordinate system to the localizer coordinate system LCLZ.

In block 604, at least one ultrasonic energy profile, such as a pulsing profile 140 or a constant energy profile 148, may be assigned to each of the virtual boundaries and/or regions. For instance, the surgical control system 562, or more particularly the navigation controller 524 as an example, may be configured to assign a pulsing profile 140 to each virtual boundary and/or region such that as the operative end 22 of the ultrasonic tip 20 reaches or crosses the virtual boundary and/or enters the region, such as indicated by the localization data generated by the localizer 522, the AC drive signal supplied to the ultrasonic instrument 18 may be set to induce pulsed ultrasonic energy according to the pulsing profile 140 assigned to the virtual boundary and/or region. In this way, the ultrasonic instrument 18 may provide varying levels of tissue selectivity and/or tactile feedback as the ultrasonic instrument 18 approaches various tissues, such as sensitive or non-target tissues near the target site TS. In some examples, the ultrasonic controller 112 may be configured to determine the available pulsing profiles 140 from the console storage 118 or the tip memory 174 as described above, and communicate such pulsing profiles 140 to the navigation controller 524 for assignment. Other arrangements in which the ultrasonic controller 112 is configured to assign pulsing profiles 140 to the virtual boundary(s) and/or region(s) are also contemplated.

In some implementations, the surgical control system 562 may be configured such that, as the operative end 22 of the ultrasonic tip 20 reaches or crosses the virtual boundary in one direction, such as the direction in which the ultrasonic instrument 18 initially reaches or crosses the virtual boundary, the AC drive signal supplied to the ultrasonic instrument 18 may be set to induce pulsed ultrasonic energy according to the pulsing profile 140 assigned to the virtual boundary. Conversely, as the operative end 22 of the ultrasonic instrument 18 reaches or crosses the virtual boundary in an opposite direction, the AC drive signal supplied to the ultrasonic instrument 18 may be set to induce the ultrasonic energy that was being induced in the ultrasonic instrument 18 prior to the initial contact with the virtual boundary. Alternatively, the surgical control system 562 may be configured to assign multiple ultrasonic energy profiles to each virtual boundary relative to travel direction such that, as the ultrasonic instrument 18 reaches or crosses the virtual boundary in one direction, the AC drive signal supplied to the ultrasonic instrument 18 may be set to induce ultrasonic energy according to one assigned ultrasonic energy profile, such as a pulsing profile 140, and as the ultrasonic instrument 18 reaches or crosses the virtual boundary in an opposite direction, the AC drive signal supplied to the ultrasonic instrument 18 may be set to induce ultrasonic energy according to another assigned ultrasonic energy profile, such as a pulsing profile 140.

In some instances, the surgical control system 562, or more particularly the navigation controller 524 as an example, may be configured to assign pulsing profiles 140 to the virtual boundary(s) and/or region(s) autonomously, such as based on the surgical procedure indicated by the surgical plan data 550 and/or the types of tissue identified in the medical image by the segmentation algorithm. For virtual region(s) identified by the segmentation algorithm as encompassing soft tissue, the surgical control system 562 may be configured to limit the assigned pulsing profiles 140 to the soft tissue pulsing profiles 144 associated with the soft tissue ablation mode of the ultrasonic tool system 12, and for virtual region(s) identified by the segmentation algorithm as encompassing hard tissue, the surgical control system 562 may be configured to limit the assigned pulsing profiles 140 to the hard tissue pulsing profiles 146 associated with the hard tissue ablation mode of the ultrasonic tool system 12. Similarly, for virtual boundary(s) identified by the segmentation algorithm as being within or adjacent soft tissue, the surgical control system 562 may be configured to limit the assigned pulsing profiles 140 to the soft tissue pulsing profiles 144 associated with the soft tissue ablation mode of the ultrasonic tool system 12, and for virtual boundary(s) identified by the segmentation algorithm as being within or adjacent hard tissue, the surgical control system 562 may be configured to limit the assigned pulsing profiles 140 to the hard tissue pulsing profiles 146 associated with the hard tissue ablation mode of the ultrasonic tool system 12.

For virtual boundary(s) identified by the segmentation algorithm as being adjacent both soft tissue and hard tissue (e.g., representing a boundary between the two types of tissue), the surgical control system 562 may be configured to limit the assigned pulsing profiles 140 based on the surgical plan data 550, or more specifically, the planned trajectory of the ultrasonic instrument 18. For instance, if the surgical plan data 550 indicates that the operative end 22 of the ultrasonic tip 20 should initially reach or cross the virtual boundary in a direction towards the soft tissue side of the boundary, the surgical control system 562 may be configured to limit the assigned pulsing profiles 140 to the soft tissue pulsing profiles 144 associated with the soft tissue ablation mode of the ultrasonic tool system 12. Conversely, if the surgical plan data 550 indicates that the operative end 22 of the ultrasonic tip 20 should initially reach or cross the virtual boundary in a direction towards the hard tissue side of the boundary, the surgical control system 562 may be configured to limit the assigned pulsing profiles 140 to the hard tissue pulsing profiles 146 associated with the hard tissue ablation mode of the ultrasonic tool system 12. Conversely, if multiple pulsing profiles 140 may be assigned to each virtual boundary relative to travel direction, then the surgical control system 562 may be configured to limit the assigned pulsing profiles 140 to the hard tissue pulsing profiles 146 associated with the hard tissue ablation mode of the ultrasonic tool system 12 relative to the direction towards the hard tissue side of the virtual boundary, and may be configured to limit the assigned pulsing profiles 140 to the soft tissue pulsing profiles 144 associated with the soft tissue ablation mode of the ultrasonic tool system 12 relative to the direction towards the soft tissue side of the virtual boundary.

Additionally or alternatively, for virtual boundary(s) and/or region(s) positioned at an edge or outside of a region of target tissue according to the surgical plan, the surgical control system 562, or more particularly the navigation controller 524 as an example, may be configured to assign pulsing profile(s) 140 providing a relatively high level of tissue selectivity and/or tactile feedback (e.g., pulse control level 4 or 5), such that the AC drive signal may be set to induce ultrasonic energy according to such pulsing profile(s) 140 as the operative end 22 of the ultrasonic tip 20 reaches or travels across these virtual boundary(s) and/or enters these virtual region(s) from the target tissue region. Conversely, for virtual boundary(s) and/or region(s) positioned within the target tissue region according to the surgical plan data 550, the surgical control system 562 may be configured to assign ultrasonic energy profiles providing a relatively low level of tissue selectivity and/or tactile feedback (e.g., pulse control level 3 or lower), such that the AC drive signal may be set to induce ultrasonic energy according to such assigned ultrasonic energy profiles as the ultrasonic instrument 18 travels across the interior virtual boundary(s) towards the edge of the target tissue region and/or enters the interior virtual region(s). In this way, as the operative end 22 moves nearer the edge of a target tissue region from within the region, the surgical control system 562, or more particularity the ultrasonic controller 112 as an example, may be configured to induce pulsed ultrasonic energy in the ultrasonic instrument 18 that provides increased tissue selectivity and/or tactile feedback to the surgeon, thereby reducing ablation in the adjacent non-target tissue region and/or causing the surgeon to take extra care when ablating tissue in the area.

In some instances, multiple virtual boundary(s)/regions(s) may be defined within the target tissue region. In this case, the surgical control system 562, or more particularity the navigation controller 524 as an example, may be configured to assign pulsing profiles 140 providing increased tissue selectivity and/or tactile feedback to the virtual boundary(s) and/or region(s) as the distance between the virtual boundary(s)/regions(s) from the edge of the target tissue region decreases.

Additionally or alternatively, the surgical control system 562, or more particularity the navigation controller 524 as an example, may be configured to allow a user to manually assign pulsing profiles 140 to the various virtual boundary(s) and/or region(s), and/or to adjust the assigned pulsing profiles 140 set by the surgical control system 562 as described above, such as via the user interface 563, or more particularly the user interface 532 of the navigation system 14 as an example. The surgical control system 562, or more particularity the navigation controller 524 as an example, may also be configured to limit the pulsing profiles 140 selectable by the user for a given boundary and/or region, such as based on the position of the virtual boundary and/or region relative to hard and soft tissue as described above. For instance, if a given boundary and/or region is located within a soft tissue region, the surgical control system 562 may be configured to limit the selectable pulsing profiles 140 to those corresponding to the soft tissue mode, and if a given boundary and/or region is located within a hard tissue region, the surgical control system 562 may be configured to limit the selectable pulsing profiles 140 to those corresponding to the hard tissue mode. Additionally or alternatively, if the surgical plan indicates the procedure is limited to ablating soft tissue or hard tissue, the surgical control system 562 may be configured to limit the selectable pulsing profiles 140 to those corresponding to the soft tissue mode or hard tissue mode as described above.

In block 606, a determination may be made of whether to activate the ultrasonic instrument 18, such as by the surgical control system 562. For instance, a user may interact with the user interface 563, or more particularly the foot pedal 76 of the ultrasonic tool system 12 as an example, to provide an instruction to the surgical control system 562, or more particularity the ultrasonic controller 112 as an example, to activate the ultrasonic instrument 18. Responsive to determining to activate the ultrasonic instrument 18 (“Yes” branch of block 606), in block 608, ultrasonic energy may be induced in the ultrasonic instrument 18. More specifically, the surgical control system 562, or more particularly the ultrasonic controller 112 as an example, may be configured to induce base ultrasonic energy in the ultrasonic instrument 18. The base ultrasonic energy may set by the practitioner, such as via the display 74 of the control console 16. In some implementations, the base ultrasonic energy may correspond to a maximum resection rate for the ultrasonic instrument 18 desired by the practitioner (e.g., constant energy profile 148, pulsing profile 140 associated with a relatively low pulsing control level).

In block 610, the pose of the ultrasonic instrument 18 in the known coordinate system may be tracked, such as based on the localization data generated by the localizer 522. More particularly, the surgical control system 562, or more particularly the navigation controller 524 as an example, may be configured to track the pose of the operative end 22 of the ultrasonic tip 20 of the ultrasonic instrument 18 relative to the virtual boundary(s) and/or region(s) in the known coordinate system, such as the localizer coordinate system LCLZ.

In block 612, ultrasonic energy, such as pulsed ultrasonic energy, may be induced in the ultrasonic instrument 18 based on the tracked pose of the ultrasonic instrument 18 and the virtual boundary(s) and/or region(s). More specifically, based on the tracked pose of the ultrasonic instrument 18 relative to the virtual boundary(s) and/or region(s), the surgical control system 562 may be configured to set the AC drive signal generated by the power supply of the ultrasonic tool system 12 to induce pulsed ultrasonic energy in the tip 20 of the ultrasonic instrument 18, such as according to the pulsing profile(s) 140 assigned to the virtual boundary(s) and/or region(s). For example, based on the tracked pose of the ultrasonic instrument 18 relative to the virtual boundary(s) and/or region(s), the navigation controller 524 may be configured to determine an assigned pulsing profile 140 to be induced in the ultrasonic instrument 18, and to communicate a corresponding message to the ultrasonic controller 112, which may be configured to responsively set the AC drive signal generated by the power supply of the ultrasonic tool system 12 to induce pulsed ultrasonic energy in the tip 20 of the ultrasonic instrument 18 according to the pulsing profile 140.

In one example, a virtual boundary may be generated that corresponds to an outside edge of a target tissue region. Based on the tracked pose of the ultrasonic instrument 18 in the known coordinate system relative to the virtual boundary, the surgical control system 562, or more particularly the navigation controller 524 as an example, may be configured to determine whether an operative end 22 of the tip 20 reaches or crosses the outside edge virtual boundary from the target tissue region. If so, then the surgical control system 562 may be configured to set the AC drive signal generated by the power supply of the ultrasonic tool system 12 to induce pulsed ultrasonic energy in the tip 20, such as according to the pulsing profile 140 assigned to the outside edge virtual boundary. For instance, the navigation controller 524 may be configured to communicate a message indicative of the assigned pulsing profile 140 to the ultrasonic controller 112, which may be configured to responsively set the AC drive signal generated by the power supply of the ultrasonic tool system 12 to induce pulsed ultrasonic energy in the tip 20 according to the pulsing profile 140. Conversely, responsive to determining that the operative end 22 of the tip 20 is within the target tissue region, the surgical control system 562 may be configured to set the AC drive signal generated by the power supply of the ultrasonic tool system 12 to induce alternative ultrasonic energy in the tip 20 (e.g., the base ultrasonic energy), such as continuous ultrasonic energy according to the constant energy profile 148 or another, typically lower level, pulsing profile 140, such as has been previously selected by the surgeon via the display 74 of the control console 16. In some examples, the induced base ultrasonic energy may provide less tissue selectivity and/or tactile feedback than any of the other assigned ultrasonic energy profiles, such as to provide a higher resection rate than the other assigned ultrasonic energy profiles.

As described above, in some examples, at least one virtual boundary may be generated within the target tissue region, with the virtual boundary being assigned a different pulsing profile 140 and being spaced a threshold distance from the outside edge virtual boundary. Based on the tracked pose of the ultrasonic instrument 18 relative to these virtual boundaries, including the outside edge virtual boundary, the surgical control system 562 may be configured to induce varying levels of pulsed ultrasonic energy in the tip 20. For instance, based on the tracked pose of the ultrasonic instrument 18 in the known coordinate system relative to the virtual boundaries, the surgical control system 562 may be configured to determine whether an operative end 22 of the tip 20 reaches or crosses the outside edge virtual boundary from the target tissue region. If so, then the surgical control system 562 may be configured to set the AC drive signal generated by the power supply of the ultrasonic tool system 12 to induce pulsed ultrasonic energy in the tip 20, such as that corresponding to the pulsing profile 140 assigned to the outside edge virtual boundary, which may be configured to provide a relatively high level of tissue selectivity and/or tactile feedback. For example, the navigation controller 524 may be configured to communicate a message indicative of the assigned pulsing profile 140 to the ultrasonic controller 112, which may be configured to set the AC drive signal generated by the power supply of the ultrasonic tool system 12 to induce pulsed ultrasonic energy in the tip 20 according to the indicated pulsing profile 140.

Moreover, based on the tracked pose of the ultrasonic instrument 18 in the known coordinate system relative to the virtual boundaries, the surgical control system 562, or more particularly the navigation controller 524 as an example, may be configured to determine whether an operative end 22 of the tip 20 is within the target tissue region with a distance between the operative end 22 of the tip 20 and the outside edge virtual boundary being less than a threshold distance, which may correspond to the virtual boundary within the target tissue region. In other words, the surgical control system 562 may be configured to determine whether the operative end 22 of the tip 20 is between the outside edge virtual boundary and the virtual boundary internal to the target tissue region. If so, then the surgical control system 562 may be configured to set the AC drive signal generated by the power supply to induce other pulsed ultrasonic energy in the tip, such as according to the pulsing profile 140 assigned to the interior virtual boundary, which may be a lower level pulsing profile 140 relative to the pulsing profile 140 assigned to the outside edge virtual boundary, and thus provide less tissue selectivity and/or tactile feedback.

In some instances, based on the tracked pose of the ultrasonic instrument 18 in the known coordinate system relative to the outside edge virtual boundary, responsive to determining that the operative end 22 of the tip 20 is within the target tissue region with a distance between the operative end 22 of the tip 20 and the outside edge virtual boundary being greater than the threshold distance associated with the interior virtual boundary, the surgical control system 562 may be configured to set the AC drive signal generated by the power supply of the ultrasonic tool system 12 to induce the base ultrasonic energy in the tip, which as described above may have been previously defined by the surgeon via the control console 16.

As discussed above, in some situations, multiple interior virtual boundaries may be generated, each at a different threshold distance from the outside edge virtual boundary. For instance and without limitation, the interior virtual boundaries may include a first interior virtual boundary a first threshold distance from the outside edge virtual boundary, and a second interior virtual boundary a second threshold distance from the outside edge virtual boundary, the second threshold distance being greater than the first threshold distance. In this case, based on the tracked pose of the ultrasonic instrument 18 in the known coordinate system relative to the virtual boundaries, the surgical control system 562 may be further configured to determine whether the operative end 22 of the tip 20 is within the target tissue region with the distance between the operative end 22 of the tip 20 and the outside edge virtual boundary being greater than the first threshold distance and less than the second threshold distance, corresponding to the operative cnd 22 of the tip 20 being between the first interior virtual boundary and the second interior virtual boundary. If so, then the surgical control system 562 may be configured to set the AC drive signal generated by the power supply to induce the pulsed ultrasonic energy in the tip 20 according to the pulsing profile 140 assigned to the second interior virtual boundary. This pulsed ultrasonic energy may differ from that induced when the operative end 22 of the 16 is between the first interior virtual boundary and the outside edge virtual boundary, such as by being a pulsing profile 140 providing less tissue selectivity and/or tactile feedback (e.g., a lower pulsing control level).

Conversely, based on the tracked pose of the ultrasonic instrument in the known coordinate system relative to the outside edge virtual boundary, responsive to determining that the operative end 22 of the tip 20 is within the target tissue region with a distance between the operative end 22 of the tip 20 and the outside edge virtual boundary being greater than the second threshold distance associated with the second interior virtual boundary, the surgical control system 562 may be configured to set the AC drive signal generated by the power supply of the ultrasonic tool system 12 to induce the base ultrasonic energy in the tip 20.

In block 614, a determination may be made of whether to deactivate the ultrasonic instrument 18. For instance, the surgeon may interact with the user interface 556 to instruct the surgical control system 562 to deactivate the ultrasonic instrument 18, such as by transitioning the foot pedal 76 to the off position. Responsive to determining that the ultrasonic instrument 18 is not to be deactivated (“No” branch of block 614), the method 600 may return to block 610 to continue tracking the pose of the ultrasonic instrument 18 in the known coordinate system, and inducing ultrasonic energy in the ultrasonic instrument 18 accordingly. Responsive to determining to deactivate the ultrasonic instrument 18 (“Yes” branch of block 614), in block 616, the ultrasonic instrument 18 may be deactivated. For instance, the surgical control system 562, or more particularly the ultrasonic controller 112 as an example, may be configured to cease the supplying of the AC drive signal to the ultrasonic instrument 18. The method 600 may then return to block 606 to monitor for reactivation of the ultrasonic instrument 18 as described above.

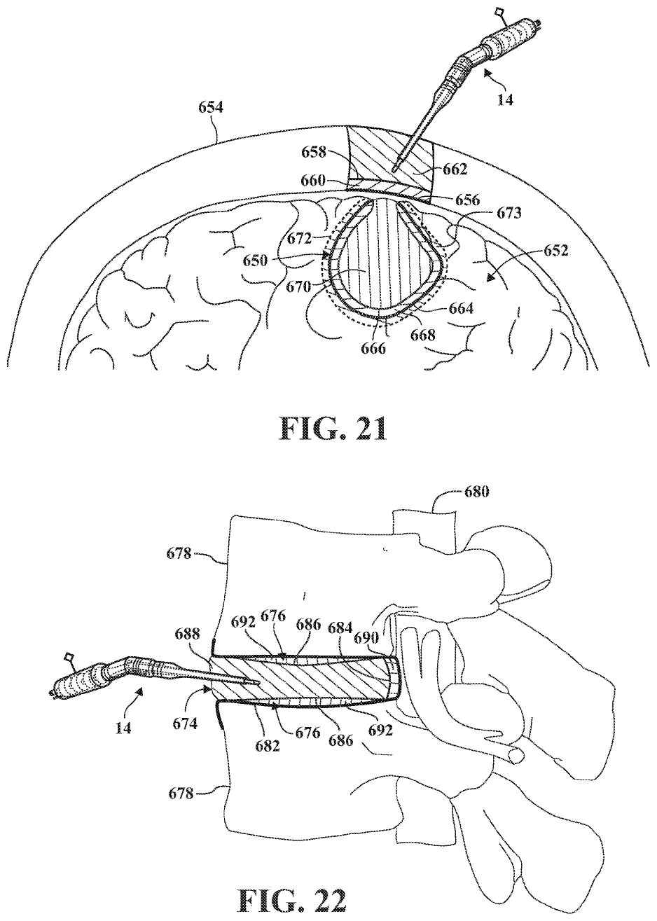

FIG. 21 illustrates virtual boundaries that may generated for a surgical procedure involving the removal of a tumorous tissue region 650 from adjacent healthy brain tissue 652. As indicated in the preloaded surgical plan data 550, the procedure may entail at least two portions, namely, a hard tissue cutting portion in which the ultrasonic instrument 18 is used to cut through the patient's skull 654, and a soft tissue ablation portion in which the ultrasonic instrument 18 is used to ablate tissue from the tumorous tissue region 650. During the hard tissue cutting portion, the surgeon may utilize a hard tissue cutting tip 20 with the intention of cutting through the skull 654 while minimizing the cutting of the healthy brain tissue 652. During the soft tissue ablation portion, the surgeon may utilize a soft tissue cutting tip 20 with the intention of removing tissue from the tumorous tissue region 650 and minimizing ablation of healthy brain tissue 652 adjacent the tumorous tissue region 650.

Relative to the hard tissue cutting portion, FIG. 21 illustrates a defined outer edge virtual boundary 656 corresponding to an inner wall of the skull 654, and corresponding to an end of a planned ablation/cutting path of the ultrasonic instrument 18 through the skull 654. FIG. 21 further illustrates an interior virtual boundary 658, which may be defined a threshold distance away from the outer edge virtual boundary 656. A virtual region 660 may be defined between the interior virtual boundary 658 and the outer edge virtual boundary 656, and another virtual region 662 may be defined between the start of the planned ablation/cutting path of the ultrasonic instrument 18 through the skull 654 and the interior virtual boundary 658. Each of the virtual boundaries 658, 656 may be associated with a different ultrasonic energy profile, and/or each of the regions 660, 662 may be associated with a different ultrasonic energy profile, such that the ultrasonic instrument 18 provides increasing tactile feedback as the operative end 22 of the tip 20 nears the end of the planned trajectory through the skull 654.

Responsive to receiving an indication to activate the ultrasonic instrument 18, such as by the surgeon actuating the foot pedal 76, the surgical control system 562 may be configured to check that the tip 20 attached to the handpiece 24 is a bone cutting tip, such as based on data stored in the tip memory 174 as described above. For instance, the ultrasonic controller 112 may be configured to read data indicative of the type of tip 20 from the tip memory 174, and to communicate a message indicative of the type of tip 20 to the navigation controller 524, which may then be configured to determine whether an appropriate tip 20 is attached based on the surgical plan data 550. If not, then the surgical control system 562 may indicate an error, such as on the user interface 556, and may prevent the ultrasonic instrument 18 from operating. Responsive to determining that a bone cutting tip 20 is affixed to the handpiece 24, the surgical control system 562 may be configured to set the AC drive signal supplied to the ultrasonic instrument 18 to induce ultrasonic energy, such as base ultrasonic energy, in the tip 20. For example, the navigation controller 524 may be configured to communicate a signal to the ultrasonic controller 112 that allows activation of the ultrasonic instrument 18. As previously discussed, the base ultrasonic energy may be continuous ultrasonic energy according to the constant energy profile 148, or pulsed ultrasonic energy according to a hard tissue pulsing profile 146 providing relatively low tactile feedback (e.g., relatively low pulse control level) that was previously selected by the surgeon. The surgical control system 562 may be configured to maintain inducement of such ultrasonic energy in the ultrasonic instrument 18 while the operative end 22 of the tip 20 is present in the region 662.

Based on the tracked pose of the ultrasonic instrument 18 in the known coordinate system, the surgical control system 562, or more particularly the navigation controller 524 as an example, may be configured to determine whether the operative end 22 of the tip 20 reaches or crosses the interior virtual boundary 658 into the virtual region 660. In other words, the surgical control system 562 may be configured to determine whether a distance between the outer edge virtual boundary 656 and the operative end 22 of the tip 20 is less than or equal to the threshold distance associated with the interior virtual boundary 658. If so, then the surgical control system 562 may be configured to set the AC drive signal to induce pulsed ultrasonic energy in the tip 20 of the ultrasonic instrument 18, such as according to a relatively high level hard tissue pulsing profile 146 (e.g., pulse control level 4) associated with the interior virtual boundary 658 and/or region 660. As a result of inducing such pulsed ultrasonic energy in the tip 20 of the ultrasonic instrument 18, the ultrasonic instrument 18 may begin providing tactile feedback of increased magnitude to alert the surgeon that the operative end 22 of the tip 20 is nearing the inner wall of the skull 654. The surgeon may then take a cue from the increased tactile feedback to reduce the force he or she applies to the ultrasonic instrument 18 and/or velocity in which the ultrasonic instrument 18 is moving through the bone, which may help the surgeon maintain control and reduce ablation of healthy adjacent tissue as the ultrasonic instrument 18 breaks through the inner wall of the skull 654.

Thereafter, based on the tracked pose of the ultrasonic instrument 18 in the known coordinate system, the surgical control system 562 may be configured to determine whether the operative end 22 of the tip 20 reaches or crosses the outer edge virtual boundary 656, indicative that the operative end 22 of the tip 20 has broken through the inner wall of the skull 654. If so, the surgical control system 562 may be configured to set the AC drive signal to induce pulsed ultrasonic energy in the tip 20 of the ultrasonic instrument 18 according to a further higher level hard tissue pulsing profile 146 (e.g., pulse control level 5) associated with the outer edge virtual boundary 656, such as to increase the level of tactile feedback felt by the surgeon to indicate that the operative end 22 of the tip has broken through. Alternatively, the surgical control system 562 may be configured to deactivate the ultrasonic instrument 18. The surgeon may then proceed to manually override the deactivation, such as by moving the ultrasonic instrument 18 backwards from its current position, in which case the surgical control system 562 may operate the ultrasonic instrument 18 based on the tracked pose of the operative end 22 of the tip 20 as described above, or such as by returning the foot pedal 76 to its off position and then moving it back to an active position, in which case the navigation controller 524 may enable the ultrasonic instrument 18 to operate according to the base ultrasonic energy, the hard tissue pulsing profile 146 assigned to the interior virtual boundary 658 and/or region 660, or another hard tissue pulsing profile 146 selected by a user.

Following completion of the bone cutting portion of the procedure, the surgeon may swap out the bone cutting tip 20 for a soft tissue cutting tip 20, and may then indicate that the soft tissue cutting portion is to be started, such as by depressing the foot pedal 76 and/or interacting with the user interface 532 of the navigation system 14. Relative to the soft tissue ablation portion of the procedure, FIG. 21 illustrates an outer edge virtual boundary 664 defined to correspond to an outer boundary of the tumorous tissue region 650, and an interior virtual boundary 666 defined to correspond to a threshold distance away from the outer edge virtual boundary 664. A virtual region 668 may be defined between the interior virtual boundary 666 and the outer edge virtual boundary 664, and another virtual region 670 may be defined between the interior virtual boundary 666 and an entry point of the planned ablation/cutting path of the ultrasonic instrument 18 into the tumorous tissue region 650. Each of the virtual boundaries 666, 664 may be associated with a different ultrasonic energy profile, such as a different soft tissue pulsing profile 144, and/or each of the regions 668, 670 may be associated with a different ultrasonic energy profile, such as a different soft tissue pulsing profile 144, such that the ultrasonic instrument 18 provides increased tactile feedback and/or tissue selectivity as the operative end 22 of the tip 20 moves nearer the boundary between the tumorous tissue region 650 and the adjacent healthy brain tissue 652.

Responsive to receiving an indication to activate the ultrasonic instrument 18 as described above, the surgical control system 562 may be configured to check that the tip 20 attached to the handpiece 24 is a soft tissue ablation tip, such as based on data stored in the tip memory as described above. If not, then the surgical control system 562 may be configured to indicate an error, such as on the user interface 556, and to prevent the ultrasonic instrument 18 from operating. Responsive to determining that a soft tissue ablation tip 20 is affixed to the handpiece 24, the surgical control system 562 may be configured to set the AC drive signal to induce base ultrasonic energy in the tip 20, such as continuous ultrasonic energy according to the constant energy profile 148 or a relatively low level soft tissue pulsing profile 144 that was previously selected by the user. The surgical control system 562 may be configured to maintain inducement of such base ablation ultrasonic energy while the operative end 22 of the tip 20 is present in the region 670.

Based on the tracked pose of the ultrasonic instrument 18 in the known coordinate system, the surgical control system 562, or more particularly navigation controller 524 as an example, may be configured to determine whether the operative end 22 of the tip 20 reaches or crosses the interior virtual boundary 666 into the region 668. In other words, the surgical control system 562 may be configured to determine whether a distance between the outer edge virtual boundary 664 and the operative end 22 of the tip 20 is less than or equal to the threshold distance associated with the interior virtual boundary 666. If so, then then surgical control system 562 may be configured to set the AC drive signal to induce pulsed ultrasonic energy in the tip 20 of the ultrasonic instrument 18, such as according to a relatively high level soft tissue pulsing profile 144 (e.g., pulse control level 3, 4) associated with the interior virtual boundary 666 and/or region 668. For instance, the navigation controller 524 may be configured to communicate a corresponding signal indicative of the soft tissue pulsing profile 144 to ultrasonic controller 112, which may be configured to responsively set the AC drive signal accordingly. As a result of inducing such pulsed ultrasonic energy in the tip 20 of the ultrasonic instrument 18, the ultrasonic instrument 18 may provide increased tissue selectivity and/or tactile feedback of an increased magnitude to alert the surgeon that the operative end 22 of the tip 20 is nearing the outer wall of the tumorous tissue region 650. The surgeon may then take a cue from the increased magnitude of tactile feedback and/or tissue selectivity to proceed with care, such as by reducing the force he or she applies to the ultrasonic instrument 18 and/or the velocity in which the ultrasonic instrument 18 is moving through the tissue, which may help the surgeon to maintain control and reduce ablation of healthy brain tissue 652 adjacent the tumorous tissue region 650.

Thereafter, based on the tracked pose of the ultrasonic instrument 18 in the known coordinate system, the surgical control system 562, or more particularly the navigation controller 524 as an example, may be configured to determine whether the operative end 22 of the tip 20 reaches or crosses the outer edge virtual boundary 656, indicative that the operative end 22 of the tip 20 is adjacent to or contacting the healthy brain tissue 652. If so, then the surgical control system 562, such as via a corresponding communication between the navigation controller 524 and the ultrasonic controller 112, may be configured to set the AC drive signal to induce pulsed ultrasonic energy in the tip 20 of the ultrasonic instrument 18 according to a further higher level soft tissue pulsing profile 144 (e.g., pulse control level 5) assigned to the outer edge virtual boundary 664, which may provide increased tissue selectivity and/or provide increased tactile feedback informing the surgeon of the ultrasonic instrument's 18 position. Alternatively, the surgical control system 562 may be configured to deactivate the ultrasonic instrument 18. The surgeon may then proceed to manually override the deactivation, such as by moving the ultrasonic instrument 18 backwards from its current position, in which case the surgical control system 562 may operate the ultrasonic instrument 18 based on the tracked pose of the operative end 22 of the tip 20 as described above, or such as by returning the foot pedal 76 to its off position and then moving it back to an active position, in which case the surgical control system 562 may enable the ultrasonic instrument 18 to operate according to the base ultrasonic energy, the soft tissue pulsing profile 144 associated with the interior virtual boundary 666 and/or region 668, or another soft tissue pulsing profile 144 selected by the practitioner.

In some examples, the defined virtual boundary(s) and/or region(s) may also include an exterior virtual boundary 672 outside the outer edge virtual boundary 664, such as at a defined distance from the outer edge virtual boundary 664, and may include a virtual region 673 defined between the outer edge virtual boundary 664 and the exterior virtual boundary 672. The exterior virtual boundary 672 and/or region 673 may correspond to a desired margin of ablation around the tumorous tissue region 650, and may thus define an overall region of tissue targeted for ablation, including both the tumorous tissue region 350 and a small amount of healthy brain tissue 652. In this case, the exterior virtual boundary 672 may be assigned a soft tissue pulsing profile 144 providing increased tissue selectivity and/or tactile feedback than the soft tissue pulsing profile 144 assigned to the outer edge virtual boundary 664 and/or the virtual region 673 (e.g., a higher pulse control level). The surgical control system 562 may thus be configured to implement the assigned soft tissue pulsing profiles 144 such that, when the operative end 22 of the tip 20 proceeds into the region 673, and then up against the exterior virtual boundary 672, the ultrasonic instrument 18 provides further increased tissue selectivity and/or tactile feedback. Alternatively, the surgical control system 562 may be configured to deactivate vibrations upon the operative end 22 of the tip reaching the exterior virtual boundary 672.

FIG. 22 illustrates virtual boundaries and/or regions that may be generated in the known coordinate system for a spinal fusion surgical procedure. During such procedure, the ultrasonic instrument 18 may be used to ablate tissue, including intervertebral disc tissue 674 and cartilage endplate tissue 676, from between vertebral bodies 678 of the spine while minimizing contact with the spinal cord 680 and ablation of tissue from the vertebral bodies 678. To this end, FIG. 22 illustrates an outer edge virtual boundary 682 corresponding to an outer boundary of the tissue targeted for ablation, including the intervertebral disc tissue 674 and cartilage endplate tissue 676. FIG. 22 further illustrates an interior virtual boundary 684 positioned a threshold distance from a portion of the outer edge virtual boundary 682 adjacent the spinal cord 680, and a pair of interior virtual boundaries 686 corresponding to the boundary between the intervertebral disc tissue 674 and cartilage endplate tissue 676. A virtual region 688 may be defined between the interior virtual boundaries 684, 686, a virtual region 690 may be defined between the interior virtual boundary 684 and the portion of the outer edge virtual boundary 682 distal to the interior virtual boundary 684 and adjacent the spinal cord 680, and a virtual region 692 may be defined between each of the interior virtual boundaries 686 and the portion of the outer edge virtual boundary 682 distal the interior virtual boundary 686 and adjacent one of the vertebral bodies 678. Each of the virtual boundaries 682, 684, 686 may be assigned a different ultrasonic energy profile, and/or each of the regions 688, 690, 692 may be assigned a different ultrasonic energy profile, such as a different soft tissue pulsing profile 144, so that the ultrasonic instrument 18 provides different levels of tissue selectivity and/or tactile feel as the operative end 22 of the tip 20 of the ultrasonic instrument 18 moves through different portions of the tissue targeted for ablation.

More particularly, responsive to receiving an indication to commence the surgery, the surgical control system 562 may be configured to check that the tip 20 attached to the handpiece 24 is a soft tissue ablation tip 20, such as based on data stored in the tip memory 174 associated with the tip 20 as described above. If not, then the surgical control system 562 may indicate an error, such as on the user interface 532, and may prevent the ultrasonic instrument 18 from operating. Responsive to determining that a soft tissue ablation tip 20 is affixed to the handpiece 24, the surgical control system 562 may be configured to set the AC drive signal to induce base ultrasonic energy in the tip 20, such as continuous ultrasonic energy according to the constant energy profile 148 or a soft tissue pulsing profile 144 previously selected by the user. The surgical control system 562 may be configured to continue inducement of the base ablation ultrasonic energy while the operative end 22 of the tip 20 is within the region 688.

Based on the tracked pose of the ultrasonic instrument 18 in the known coordinate system, the surgical control system 562, or more particularly the navigation controller 524 as an example, may be configured to determine whether the operative end 22 of the tip 20 reaches or crosses the interior virtual boundary 684 into the region 690. In other words, the surgical control system 562 may be configured to determine whether a distance between the portion of the outer edge virtual boundary 682 adjacent the spinal cord 680 and the operative end 22 of the tip 20 is less than or equal to the threshold distance associated with the interior virtual boundary 684. If so, then the surgical control system 562 may be configured to set the AC drive signal to induce pulsed ultrasonic energy in the tip 20 of the ultrasonic instrument 18, such as according to a soft tissue pulsing profile 144 associated with the interior virtual boundary 684 and/or region 690, which may differ from the base ultrasonic energy such as by offering increased tissue selectivity and/or tactile feedback (e.g., pulse control level 4). The surgeon may then take a cue from the increased magnitude of tactile feedback and/or tissue selectivity to reduce the force he or she applies to the ultrasonic instrument 18 and/or velocity in which the ultrasonic instrument 18 is moving through the tissue, which may help the surgeon maintain control and reduce trauma to adjacent tissue not targeted for ablation and contact with the spinal cord 680.

Based on the tracked pose of the ultrasonic instrument 18 in the known coordinate system, the surgical control system 562 may also be configured to determine whether the operative end 22 of the tip 20 reaches or crosses the portion of the outer edge virtual boundary 682 adjacent the spinal cord 680, indicative that the operative end 22 of the tip 20 has broken through a distal end of the intervertebral disc tissue 674. If so, then the surgical control system 562 may be configured to set the AC drive signal to induce pulsed ultrasonic energy in the tip 20 of the ultrasonic instrument 18 according to a soft tissue pulsing profile 144 associated with the outer edge virtual boundary 682, which may differ from the previously induced soft tissue pulsing profile 144 and may provide further tissue selectivity and/or tactile feedback (e.g., pulse control level 5). Alternatively, the surgical control system 562 may be configured to deactivate the ultrasonic instrument 18. The surgeon may then proceed to manually override the deactivation, such as by moving the ultrasonic instrument 18 backwards from its current position, in which case the surgical control system 562 may operate the ultrasonic instrument 18 based on the tracked pose of the operative end 22 of the tip 20 as described above, or such as by returning the foot pedal 76 to its off position and then moving it back to an active position, in which case the surgical control system 562 may enable the ultrasonic instrument 18 to operate according to the base ultrasonic energy, the soft tissue pulsing profile 144 assigned to the outer edge virtual boundary 682, or another pulsing profile 140 selected by the user.

Based on the tracked pose of the ultrasonic instrument 18 in the known coordinate system, the surgical control system 562 may also be configured to determine whether the operative end 22 of the tip 20 reaches or crosses one of the interior virtual boundaries 686 into the region 692 from the region 688. If so, then the navigation controller 524 may be configured to set the AC drive signal to induce pulsed ultrasonic energy in the tip 20 of the ultrasonic instrument 18, such as according to a soft tissue pulsing profile 144 assigned to the interior virtual boundary 686 and/or region 692, which may differ from the base ultrasonic energy by providing increased tissue selectivity and/or tactile feedback (e.g., pulse control level 4). The increased tissue selectivity and/or tactile feedback may alert the surgeon that the operative end 22 of the tip 20 is contacting the cartilage endplate tissue 676 near the vertebral body 678. The surgeon may take a cue from the increased tissue selectivity and/or tactile feedback to reduce the force he or she applies to the ultrasonic instrument 18 and/or velocity in which the ultrasonic instrument 18 is moving through the tissue, which may help the surgeon maintain control and reduce trauma to the tissue not targeted for ablation.

Based on the tracked pose of the ultrasonic instrument 18 in the known coordinate system, the surgical control system 562 may further be configured to determine whether the operative end 22 of the tip 20 reaches or crosses the portion of the outer edge virtual boundary 682 adjacent the vertebral body 678 from the region 692, indicative that the operative end 22 of the tip 20 is contacting or near contacting the vertebral body 678. If so, then the surgical control system 562 may be configured to set the AC drive signal to induce pulsed ultrasonic energy in the tip 20 of the ultrasonic instrument 18 according to a soft tissue pulsing profile 144 assigned to the portion of the outer edge virtual boundary 682, which may differ from and may provide further tissue selectivity and tactile feedback than the pulsing profile 144 assigned to the interior virtual boundary 686 and/or region 692 (e.g., pulse control level 5), thereby alerting the surgeon to back the operative end 22 of the tip 20 off from the current position. Alternatively, the navigation controller 524 may be configured to deactivate the ultrasonic instrument 18. The surgeon may then proceed to override the deactivation, such as by moving the ultrasonic instrument 18 backwards from its current position, in which case the surgical control system 562 may operate the ultrasonic instrument 18 based on the tracked pose of the operative end 22 of the tip 20 as described above, or by moving the foot pedal 76 to its off position and then back to an active position, in which case the surgical control system 562 may enable the ultrasonic instrument 18 to operate according to the base ultrasonic energy, the soft tissue pulsing profile 144 corresponding to the outer edge virtual boundary 682, or another soft tissue pulsing profile 144 selected by the user.

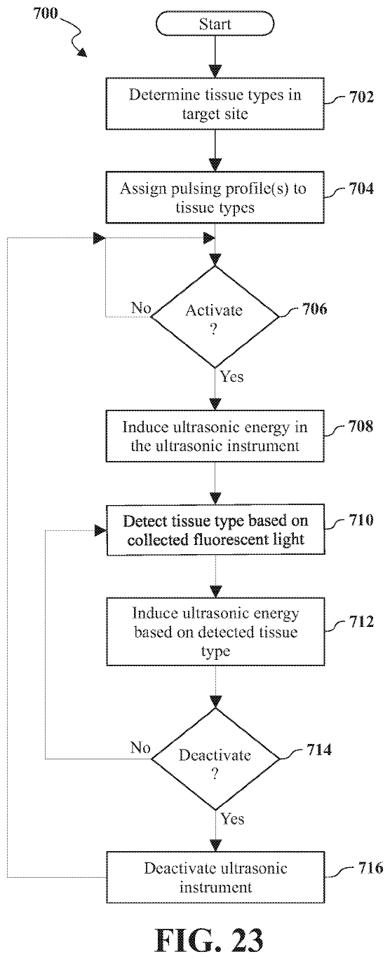

FIG. 23 illustrates a method 700 for operating the ultrasonic instrument 18 to resect tissue at a target site including one or more types of tissue based on a tissue type detected by the tissue detection system 13. The method 700 may be implemented by the surgical control system 562, or more particularly by the tissue detection controller 302 and the ultrasonic controller 112 as an example.

In block 702, the types of tissue in or adjacent the target site may be determined. For instance, a practitioner may interact with the user interface 556, or more particularly the display 104 of the control console 86 of the tissue detection system 13 as an example, to select preloaded tissue types, which may be stored as tissue map data 320 in the tissue detection console storage 318. Additionally or alternatively, the surgical control system 562, or more particularly the tissue detection controller 302 as an example, may prompt the practitioner, such as via the display 104, to place the distal portion of the sample element 88, such as when coupled to the ultrasonic instrument 18, adjacent each type of tissue in or near the target site multiple times and/or at multiple locations.

Responsive to receiving an indication from the practitioner, such as via the display 104, that the sample element 88 is positioned adjacent a given type of tissue, the tissue detection controller 302 may be configured to illuminate the tissue adjacent the distal portion of the sample element 88 and collect the resulting fluorescent light as described above. For each type of tissue to be detected during a surgical procedure, the surgical control system 562, or more particularly the tissue detection controller 302 as an example, may then be configured to analyze the instances of fluorescence light collected for the type of tissue to determine sample light intensities corresponding to each of one or more fluorophores associated the type of tissue. The surgical control system 562 may then be configured to generate tissue map data 320 for the type of tissue that indicates a minimum threshold value for each of the one or more fluorophores. For instance and without limitation, the minimum threshold value for each fluorophore may be based on the lowest intensity sample for the fluorophore, or on an average of a fixed number of lowest intensity samples for the fluorophore. As an example, the tissue detection controller 302 may be configured to determine the minimum intensity threshold for each fluorophore by subtracting a predetermined buffer value from the lowest intensity value or the average.

At block 704, an ultrasonic energy profile, such as the constant energy profile 148 or a pulsing profile 140, may be assigned to each type of tissue. For instance, the surgical control system 562, or more particularly the tissue detection controller 302 or the ultrasonic controller 112 as an example, may be configured to assign a pulsing profile 140 to each type of tissue such that, when the operative end 22 of the tip 20 of the ultrasonic instrument 18 contacts the type of tissue, the AC drive signal supplied to the ultrasonic instrument 18 is set to induce pulsed ultrasonic energy according to the pulsing profile 140 assigned to the type of tissue. In this way, the ultrasonic instrument 18 may provide varying levels of tissue selectivity and/or tactile feedback as the ultrasonic instrument 18 contacts various tissue types, such as tissue types corresponding to or being near sensitive or non-target tissues in or near the target site TS.

In some implementations, the surgical control system 562, or more particularly the tissue detection controller 302 or the ultrasonic controller 112 as an example, may be configured to assign pulsing profiles 140 autonomously, such as based on the determined tissue types and data included in the tissue map data 320 and/or tissue type data 142 indicative of which pulsing profile 140 to assign to each of various tissue types. Additionally or alternatively, the surgical control system 562 may be configured to leverage localization data generated by the localizer 522 to assign pulsing profiles 140 as described above. When assigning the pulsing profiles 140, the surgical control system 562 may also be configured to limit which pulsing profiles 140 may be assigned to a given type of tissue as described above, such as based on whether the type of tissue is hard or soft tissue. Additionally or alternatively, the surgical control system 562 may be configured to enable a user to modify the assigned or assign pulsing profiles 140 via the user interface 556.

At block 706, a determination may be made of whether to activate the ultrasonic instrument 18. For instance, a practitioner may move the foot pedal 76 from the off position to an active position to provide an indication to activate the ultrasonic instrument. Responsive to determining to activate the ultrasonic instrument 18 (“Yes” branch of block 706), in block 708, ultrasonic energy may be induced in the ultrasonic instrument 18, such as by the surgical control system 562. For instance, the ultrasonic controller 112 may be configured to induce base ultrasonic energy in the ultrasonic instrument 18, which may correspond to an ultrasonic energy profile and power setting selected by the practitioner for the base ultrasonic energy, such as via the display 74 of the control console 16.

In block 710, the type of tissue being contacted by the operative end 22 of the tip 20 of the ultrasonic instrument 18 may be detected. For example, the surgical control system 562, or more particularly the tissue detection controller 302 as an example, may be configured to illuminate the tissue with excitation light at one or more wavelengths via the excitation fiber 100, and to collect resulting fluorescent light via the excitation fiber 94 as described above. Based on the fluorescent light, the surgical control system 562, or more particularly the tissue detection controller 302 as an example, may be configured to determine the type of tissue being contacted by the operative end 22 of the ultrasonic tip 20.

In block 712, ultrasonic energy, such as pulsed ultrasonic, may be induced in the ultrasonic instrument 18 based on the detected type of tissue, such as by the surgical control system 562. For instance, assuming a given pulsing profile 140 has been assigned to the detected type of tissue, the AC drive signal generated by the power supply of the ultrasonic tool system 12 may be set to induce pulsed ultrasonic energy in the tip according to the pulsing profile 140. In one example, the tissue detection controller 302 may be configured to communicate a message to the ultrasonic controller 112 indicative of the detected type of tissue, and the ultrasonic controller 112 may be configured to induce ultrasonic energy in the ultrasonic instrument 18 as described above based on the indicated type of tissue. Alternatively, the tissue detection controller 302 may be configured to determine the ultrasonic energy profile assigned to the detected type of tissue, and communicate a message indicating the same to the ultrasonic controller 112 for implementation.

An example of inducing ultrasonic energy based on the detected type of tissue may be described in reference to the tumor resection procedure illustrated in FIG. 21. As previously described, the target site TS in the illustrated tumor resection procedure may at least in part include a tumorous tissue region 650 and adjacent healthy brain tissue 652. The tumorous tissue region 650 may be marked by the surgical control system 562 as a type of tissue targeted for ablation, and the adjacent healthy brain tissue 652 may be marked as a type of tissue not targeted for ablation. These tissues may be detectable by the surgical control system 562, or more particularly the tissue detection system 13 as an example, and may each be assigned an ultrasonic energy profile. For instance, the type of tissue targeted for ablation may be assigned the constant energy profile 148 or a soft tissue pulsing profile 144 offering relatively low tissue selectivity and/or tactile feedback (e.g., pulse control level 1), and the type of tissue not targeted for ablation may be assigned a soft tissue pulsing profile 144 providing relatively high tissue selectivity and/or tactile feedback. In this way, as the operative end 22 moves from contacting the tumorous tissue region 650 to the adjacent healthy brain tissue 652, the ultrasonic instrument 18 may provide increased tissue selectivity, thereby minimizing undesired ablation of the healthy brain tissue 652. The ultrasonic instrument 18 may also provide increased tactile feedback, which may be perceived by and indicate to the surgeon that the operative end 22 of the ultrasonic tip 20 is contacting the healthy brain tissue 652. The surgeon may take such tactile feedback and/or increase in tissue selectivity as a cue to proceed with additional caution.

In some examples, a target site TS may include a region of a type of tissue in which it may be desirable to induce varying ultrasonic energy across the region. For instance, relative to a region of a type of tissue targeted for ablation that is adjacent a region of a type of tissue that is not targeted for ablation, it may be desirable to induce pulsed ultrasonic energy of increased tissue selectivity and/or tactile feedback as the operative end 22 of the ultrasonic tip 20 moves towards the periphery of the region. In this way, as the operative end 22 of the ultrasonic tip 20 moves nearer the edge of the region of the type of tissue targeted for ablation, the ultrasonic instrument 18 may provide increased tissue selectivity and/or tactile feedback, which may minimize undesired ablation to the type of tissue not targeted for ablation, and may cue to the practitioner of the location of the operative end 22 of the ultrasonic instrument 18 and to proceed with caution.

For instance, referring to the example illustrated in FIG. 21, relative to the tumorous tissue region 650, it may be desirable to induce ultrasonic energy with relatively less tissue selectivity and/or tactile feedback when the operative end 22 of the ultrasonic instrument 18 is in a center portion of the tumorous tissue region 650, such as the region 670. Conversely, it may be desirably to induce ultrasonic energy with relatively high tissue selectivity and/or tactile feedback in a portion of the tumorous tissue region 650 that is proximate another type of tissue and between the center portion and the another type of tissue, such as the periphery portion of the tumorous tissue region 650, represented by the region 668.

To this end, the center portion of the tumorous tissue region 650 may be assigned an ultrasonic energy profile with relatively low tissue selectivity and/or tactile feedback, such as the constant energy profile 148 or a relatively low level soft tissue pulsing profile 144 (e.g., pulse control level 1). Conversely, the periphery portion of the tumorous tissue region 650 may be assigned a soft tissue pulsing profile 144 offering higher tissue selectivity and/or tactile feedback, such as pulse control level 3 or 4. The region of healthy brain tissue 652 may be assigned a soft tissue pulsing profile 144 providing even further tissue selectivity and/or tactile feedback, such as pulse control level 5.

Similarly, relative to the region of healthy brain tissue 652, it may be desirable to induce ultrasonic energy with more tissue selectivity and/or tactile feedback than that induced in the tumorous tissue region 650 when the operative end 22 of the ultrasonic instrument 18 is in the portion represented by region 673, namely the portion of healthy brain tissue 652 adjacent or within a threshold distance of the tumorous tissue region 650, and to further increase tissue selectivity and/or tactile feedback of the induced ultrasonic energy as the operative end 22 of the tip 20 moves further into the healthy brain tissue 652. The portions of the healthy brain tissue 652 nearer and further from the tumorous tissue region 650 may thus be assigned soft tissue pulsing profiles 144 providing increased tissue selectivity and/or tactile feedback from that induced in tumorous tissue region 650, such as pulse control levels 4 and 5 respectively.

In some implementations, the surgical control system 562, or more particularly the tissue detection controller 302 as an example, may be configured to determine the location of the operative end 22 of the ultrasonic tip 20 relative to the edge of a region of a type of tissue based on characteristics of the collected fluorescent light. Specifically, for a region of a given type of tissue, the density of the tissue, and the density of the fluorophores within the tissue, may decrease towards the edge of the region, causing a change to the characteristics of the fluorescent light emitted from the region as the distal portion of the sample element 88 moves from a central portion towards the edge of the region. In other words, as the distal portion of the sample element 88 moves from the central portion to the edge of the region, the intensity of the wavelengths of fluorescent light specific to the type of tissue may vary, such as according to a gradient from greater intensity to lesser intensity.

Thus, in order to detect different portions of a region of a given type of tissue, the tissue type data 105 stored in the tissue detection console storage 318 may indicate both a minimum intensity threshold corresponding to the type of tissue, and one or more other intensity thresholds greater than the minimum intensity threshold for distinguishing different portions of the region of the type of tissue. For example, the one or more other intensity thresholds may include a periphery intensity threshold such that, if a measured fluorescent intensity at a wavelength corresponding to the tissue type is greater than the minimum intensity threshold and greater than the periphery intensity threshold, then the surgical control system 562, or more particularly the tissue detection controller 302, may be configured to determine that the operative end 22 of the ultrasonic tip 20 is within a center portion of the region. Alternatively, if the measured fluorescent intensity at a wavelength corresponding to the tissue type is greater than the minimum intensity threshold and less than or equal to the periphery intensity threshold, then the surgical control system 562, or more particularly the tissue detection controller 302, may be configured to determine that the operative end 22 of the ultrasonic tip 20 is in a periphery portion of the region, which may correspond to a portion of the region that is within a threshold distance from the edge of the region. In some examples, the tissue type data 105 may also include one or more further intensity threshold values greater than the periphery intensity threshold value and corresponding to further bands between the periphery portion and the central portion of the region. Each of these bands may likewise be assigned a pulsing profile 140 such that the tissue selectivity and/or tactile feedback provided by the ultrasonic instrument 18 is increased as the operative end 22 of the tip 20 moves through the bands in a direction according to a planned ablation path.

In addition or alternatively, the surgical control system 562, such as the ultrasonic controller 112 or the tissue detection controller 302 as an example, may be configured to leverage characteristics of the AC drive signal to determine the portion of a region of a given type of tissue in which the operative end 22 of the ultrasonic tip 20 is located. Specifically, for a region of a given type of tissue, the density of the tissue may decrease, and correspondingly the mechanical impedance (e.g., stiffness) of the tissue may decrease. As described above, the ultrasonic controller 112 may be configured to determine the load applied to the ultrasonic instrument 18, which may be a function of the stiffness of the tissue being contacted by the operative end 22 of the tip 20, based on a load measurement value. The load measurement may be determined based on characteristics of the AC drive signal, such as based on the voltage vₛ of the AC drive signal, or based on the voltage vₛ and current iₛ of the AC drive signal (e.g., by calculating the mechanical resistance R_{M} of the ultrasonic instrument 18).

The tissue type data 142, such as stored in the ultrasonic console storage 118, may include data indicating various tissue types and a range of load measurement values corresponding to each of the tissue types, with different values within the range being associated with different portions of a region of the tissue type. For instance, for a given tissue type, the tissue type data 142 may indicate a load threshold value such that a determined load measurement value being greater than the load threshold value indicates the operative end 22 of the ultrasonic tip 20 is contacting a central portion of a region of the given type of tissue, and the determined load measurement value being less than the load threshold value indicates that the operative end 22 of the ultrasonic tip 20 is contacting a peripheral portion of the region, which may correspond to a portion of the region that is within a threshold distance from the edge of the region. In some examples, the tissue type data 142 may also include one or more further load threshold values greater than the periphery load threshold value and corresponding to further bands between the periphery portion and the central portion of the region. Each of these bands may likewise be assigned a pulsing profile 140 such that the tissue selectivity and/or tactile feedback provided by the ultrasonic instrument 18 increases as the operative end 22 of the tip 20 moves through the bands in a direction according to a planned ablation path.

Referring again to FIG. 23, in block 714, a determination may be made of whether to deactivate the ultrasonic instrument 18. For instance, the practitioner may instruct the surgical control system 562 to deactivate the ultrasonic instrument 18 by transitioning the foot pedal 76 to the off position. Responsive to determining that the ultrasonic instrument 18 is not to be deactivated (“No” branch of block 714), the method 700 may return to block 710 to continue detecting the type of tissue adjacent the operative end 22 of the ultrasonic tip 20, and inducing ultrasonic energy in the ultrasonic instrument 18 accordingly. Responsive to determining to deactivate the ultrasonic instrument 18 (“Yes” branch of block 714), in block 716, the ultrasonic instrument 18 may be deactivated. For instance, the control console 16 of the ultrasonic tool system 12 may be configured to cease supplying the AC drive signal to the ultrasonic instrument 18. The method 700 may then return to block 706 to monitor for reactivation of the ultrasonic instrument 18 as described above.

As mentioned above, different tips 20 removably coupleable to the handpiece 24 may be configured for different types of operations, such as soft tissue ablation or hard tissue ablation. In one example, the tip memory 174 distributed with a given tip 20 may indicate the type of tissues intended for the tip 20. In some implementations, responsive to the surgical control system 562, or more particularly the tissue detection system 13 as an example, generating tissue contact data indicating that the currently contacted tissue is of a type incompatible with the tip 20 (e.g., a soft tissue tip 20 contacts hard tissue, a hard tissue tip 20 contacts soft tissue), the surgical control system 562, such as via the ultrasonic controller 112, may be configured to cease vibrations of the ultrasonic instrument 18, and prompt a user via the user interface 563 to swap the current tip 20 with an alternative tip 20 configured for the type of contacted tissue.

Additionally or alternatively, some tips 20 may be configured for both ablating soft tissue and cutting relatively hard tissues. In this case, when such a tip 20 is incorporated in the ultrasonic instrument 18, responsive to the surgical control system 562 generating tissue contact data indicating that the ultrasonic instrument 18 transitions to contacting a tissue of a different type (e.g., from soft to hard tissue, or vice versa), the surgical control system 562, or more particularly the ultrasonic controller 112 as an example, may be configured to automatically modify the ultrasonic energy induced in the ultrasonic instrument 18 in accordance with the different tissue type. For example, the surgical control system 562 may be configured to automatically switch between inducing soft tissue pulsing profiles 144 and hard tissue pulsing profiles 146 in the ultrasonic instrument 18 as appropriate, such as according to the current pulsing profile 140 assigned to the type of contacted tissue, or to a virtual boundary, region, or stiffness level associated with the contacted tissue. Additionally or alternatively, the surgical control system 562 may be configured, such as via the ultrasonic controller 112, to automatically vary the maximum ultrasonic energy level induced in the ultrasonic instrument 18 based on the type of tissue currently being contacted, such as according to a unique maximum ultrasonic energy level that may be assigned to the type of contacted tissue, or a virtual boundary, virtual region, or stiffness level associated with the contacted tissue, by the surgical control system 562 and/or the practitioner using the user interface 563.

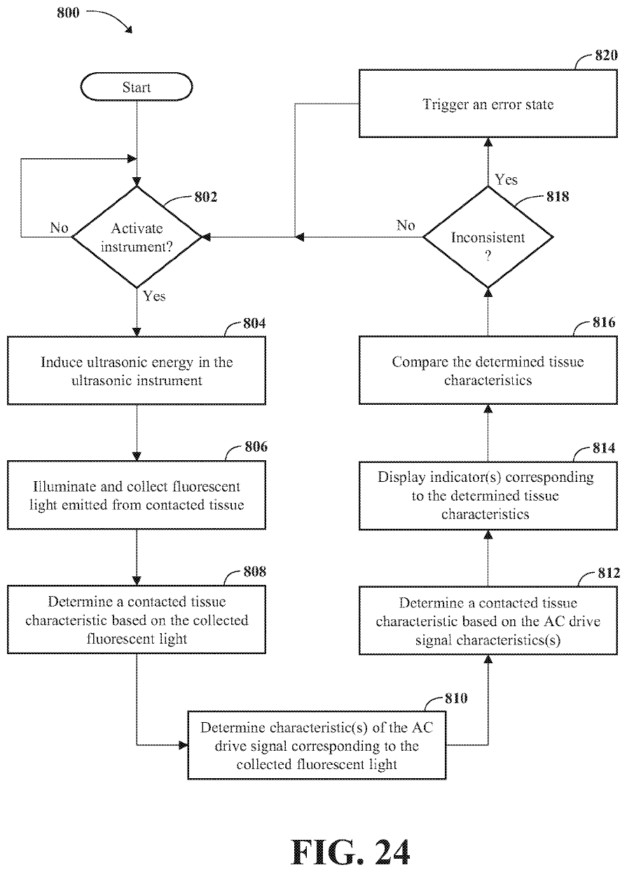

FIG. 24 illustrates a method 800 for verifying tissue detection during a surgical procedure. The method 800 may be implemented by the surgical control system 562, or more particularly by one or more of the controllers 112, 302, 524 of the surgical system 10.

In block 802, a determination may be made of whether to activate the ultrasonic instrument 18, such as by the surgical control system 562, or more particularly the ultrasonic controller 112 as an example. For instance, a user may depress the foot pedal 76 of the ultrasonic tool system 12, which may communicate a signal to the ultrasonic controller 112 indicative of the depression. Responsive to receiving such signal, the ultrasonic controller 112 may be configured to determine to activate the ultrasonic instrument 18. Responsive to determining to activate the ultrasonic instrument 18 (“Yes” branch of block 802), in block 804, ultrasonic energy may be induced in the ultrasonic instrument 18. More specifically, the ultrasonic controller 112 may be configured to communicate control signals to the signal generator 114 of the ultrasonic control console 16 in accordance with the current settings of the ultrasonic tool system 12 and/or based on the extent of the depression of the foot pedal 76. The signal generator 114 may be configured to responsively generate an AC drive signal based on the control signals as described above, which may be applied to and induce ultrasonic energy in the ultrasonic instrument 18. As long as the ultrasonic energy is being induced in ultrasonic instrument 18, the ultrasonic controller 112 may be configured to adjust the control signals based on received feedback regarding the AC drive signal for maintaining the induced ultrasonic energy at a target level and frequency as described above.

In block 806, the tissue being contacted the operative end 22 of the tip 20 may be illuminated with excitation light, and fluorescent light emitted from the tissue responsive to the excitation light may be collected. More specifically, the surgical control system 562, or more particularly the tissue detection controller 302 as an example, may be configured to cause the excitation sources(s) 312 to emit excitation light, which may be guided by the optics block 308 down to the distal region 98 of the excitation fiber 94 and into the tissue being contacted by the operative end 22 of the tip 20. Fluorescent light emitted from the tissue in response to the excitation light may then be collected by the excitation fiber 94, which may then be converted by the spectrometer 310 into spectral signals at the instruction of the tissue detection controller 302 and provided to the tissue detection controller 302 for analysis.

In block 808, a characteristic of the contacted tissue may be determined based on the collected fluorescent light, or more particularly based on the spectral signals derived from the collected fluorescent light. For example, the surgical control system 562, or more particularly the tissue detection controller 302 as an example, may be configured to determine a tissue characteristic of the contacted tissue, such as by accessing the tissue map data 320 that indicates a tissue characteristic as a function of one or more characteristics of the spectral signals (e.g., intensity, frequency). As one example, the tissue characteristic indicated by the tissue map data 320 may indicate a type of tissue being contacted by the operative end 22 of the tip 20, such as whether the contacted tissue is a healthy type of tissue or tumorous tissue. The determined tissue characteristic may also indicate whether the contacted tissue is targeted for ablation or non-targeted.

In block 810, one or more characteristics of the AC drive signal corresponding to the collected fluorescent light indicative of the tissue characteristic determined in block 808 may be determined, such as by the surgical control system 562, or more particularly by the ultrasonic controller 112. The determined characteristic(s) of the AC drive signal may correspond in time with the collection of the fluorescent light, such that the AC drive signal includes the determined characteristic(s) contemporaneously with the sample element 88 collecting fluorescent light from the contacted tissue.

In block 812, a characteristic of the tissue being contacted by the operative end 22 of the tip 20 may be determined based on determined characteristic(s) of the AC drive signal. More particularly, the ultrasonic controller 112 may be configured to, based on the determined AC drive signal characteristic(s), determine a characteristic of the tissue being contacted by the operative end 22 of the tip 20 that is indicated by the AC drive signal.

The determined characteristic(s) of the AC drive signal may generally relate or vary relative to one or more characteristic(s) of the tissue being contacted by the operative end 22 of the tip 20, such as a mechanical impedance of the tissue, which may be a function of one or more of the mass, spring, and damping characteristics of such tissue. In one example, the determined characteristic(s) of the AC drive signal may correspond to a stiffness of the contacted tissue. The mechanical impedance or stiffness of tissue indicated by the determined characteristic(s) of the AC drive signal may likewise vary as a function of the type of contacted tissue (e.g., in soft tissue, tumorous tissue may be stiffer than healthy, non-tumorous tissue). Accordingly, the determined characteristic(s) of the AC drive signal may indicate a type of tissue being contacted by the operative end 22 of the tip 20, and may correspondingly indicate whether the contacted tissue is targeted for ablation or non-targeted.

For example and without limitation, the determined characteristic(s) may include a measured voltage vₛ of the AC drive signal, determined by the ultrasonic controller 112 using the voltage measuring circuit 230 as described above. During operation of the ultrasonic instrument 18, the ultrasonic controller 112 may be configured to adjust the voltage vₛ of the AC drive signal to maintain a target mechanical current i_{M} through the ultrasonic instrument 18. As the load on the operative end 22 of the tip 20 varies, the mechanical impedance Z_{M}, or more particularly the resistive component R_{M} of the mechanical impedance Z_{M}, may also vary. Correspondingly, the mechanical current i_{M} induced in the ultrasonic instrument 18 may vary, causing the ultrasonic controller 112 to vary the voltage vₛ of the AC drive signal so as to maintain the mechanical current i_{M} at a target level. As different tissues may place different loads on the tip due to their varying mechanical properties (e.g., stiffness), the voltage vₛ of the AC drive signal may thus be a function of the characteristics or type of the tissue against which the operative end 22 of the tip 20 is vibrating.

Accordingly, the ultrasonic controller 112 may be configured to use the measured voltage v's of the AC drive signal as indicative of one or more characteristics of the contacted tissue. More specifically, the ultrasonic controller 112 may be configured to query the measured voltage vₛ of the AC drive signal against the tissue type data 142, which may associate one or more (e.g., a range) of predetermined voltage values with each of varying tissue characteristics, to determine a tissue characteristic indicated by the one or more characteristics of the AC drive signal corresponding to the collected fluorescent data.

As another example, the determined characteristic(s) of the AC drive signal may include both the measured voltage vₛ and the measured current iₛ of the AC drive signal, and the ultrasonic controller 112 may be configured to calculate the resistive component R_{M} of the mechanical impedance Z_{M} of the ultrasonic instrument 18 (also referred to as mechanical resistance R_{M}) to determine the characteristic of the contacted tissue. More specifically, because the mechanical impedance Z_{M} of the ultrasonic instrument 18 is equal to the mechanical resistance R_{M} when the ultrasonic instrument 18 is operating at resonance (e.g., the reactive components cancel each other out), the ultrasonic controller 112 may be configured to calculate the mechanical resistance R_{M} by calculating the mechanical current i_{M} as described in Applicant's U.S. Pat. No. 10,016,209, and dividing the measured voltage vₛ of the AC drive signal by the calculated mechanical current i_{M}. The ultrasonic controller 112 may then be configured to query the mechanical resistance R_{M} against the tissue type data 142, which in this case may associate one or more (e.g., a range) of predetermined resistance values with each of varying tissue characteristics, to determine a tissue characteristic or type indicated by the one or more characteristics of the AC drive signal corresponding to the collected fluorescent data.

In some instances, the ultrasonic controller 112 may also be configured to calibrate the comparison of the one or more characteristics of the AC drive signal with the tissue type data 142 based on the personal habits of the practitioner using the ultrasonic instrument 18, and/or based on the particular handpiece 24 and tip 20 combination. For example, different practitioners may apply more or less force to the ultrasonic instrument 18 when ablating tissue, which may likewise affect the load on the operative end 22 of the tip 20. Accordingly, to tailor the above tissue characteristic determination to the specific practitioner, prior to the surgical procedure, the ultrasonic controller 112 may be configured to prompt the practitioner, such as via the display 74, to apply the vibrating operative end 22 of the tip 20 against various artificial simulations or samples of tissues of varying characteristics involved in the surgical procedure. The ultrasonic controller 112 may then be configured to determine an offset value (e.g., voltage offset value or mechanical resistance offset value) based on the differences between expected values related to the determined AC drive signal characteristic(s) (e.g., voltages vₛ or mechanical resistances R_{M} of the ultrasonic instrument 18) for the various tissues and the measured values related to the determined AC drive signal characteristic(s) when the practitioner is applying the operative end 22 of the tip 20 to the simulations or samples of the tissues. For instance, the offset value may be set to an average of the differences. Later, in block 812, the ultrasonic controller 112 may be configured to apply (e.g., add or subtract) the determined offset value to the measured value (e.g., voltage vₛ or calculated mechanical resistance vₛ and compare the result to the previously stored tissue type data 142 to determine a characteristic or type of the contacted tissue indicated by the characteristic(s) of the AC drive signal.

Additionally or alternatively, referring back to FIG. 5, the HP memory 168 and/or tip memory 174 may each store data indicative of voltage or mechanical resistance offset value specific to the handpiece 24 and/or tip 20. Specifically, varying versions of these components may provide varying levels of impedance on the ultrasonic instrument 18 during vibration, which may in turn vary the voltage vₛ and mechanical resistances R_{M} indicated by the AC drive signal when the component is vibrated to ablate tissue. Accordingly, an offset specific to each component may be predetermined and stored in the relevant memory 168, 174 by operating the component version in free air (i.e., not in contact with any tissue) and calculating the voltage vₛ or mechanical resistance R_{M} indicated by the AC drive signal. These values may then be stored in the memory 168, 174 of the component to be used as an offset in block 812. In other words, responsive to determining a voltage vₛ or mechanical resistance R_{M} of the ultrasonic instrument 18 in block 812, the ultrasonic controller 112 may be configured to reduce the voltage vₛ or mechanical resistance R_{M} by the corresponding offset stored in the HP memory 168 and/or by the corresponding offset stored in the tip memory 174, and compare the result against the tissue type data 142 to determine the tissue characteristic indicated by the characteristic(s) of the AC drive signal.

Additionally or alternatively, the ultrasonic controller 112 may also be configured to calibrate the comparison based on the level (e.g., flow rate) of irrigating fluid being provided via the sleeve 42, which may be set by the practitioner via the control console 16, and may affect the load on the mechanical components of the ultrasonic instrument 18, including the tip 20. The console storage 118, such as the tissue type data 142, may thus include data indicating values by which to offset (e.g., reduce) the values related to the characteristic(s) AC drive signal that are described above for different irrigating fluid levels being implemented through the ultrasonic instrument 18.

In some implementations, the characteristic(s) of the AC drive signal that are indicative of the contacted type of tissue may also be determined as described in Applicant's PCT Publication No. WO 2021/248062 A1, the contents of which are hereby incorporated herein by reference in their entirety.

In block 816, at least one indicator corresponding to the determined tissue characteristics may be displayed. More specifically, the surgical control system 562, or more particularly one of the controllers 112, 302, may be configured to receive the tissue characteristic determined by the other controller 112, 302, and to display an indicator corresponding to the tissue characteristic determined by the tissue detection system 13 and an indicator corresponding to the tissue characteristic determined by the ultrasonic tool system 12. Such indicators may be shown on the display 74, 104 corresponding to the controller 112, 302 causing the indicators to be displayed.

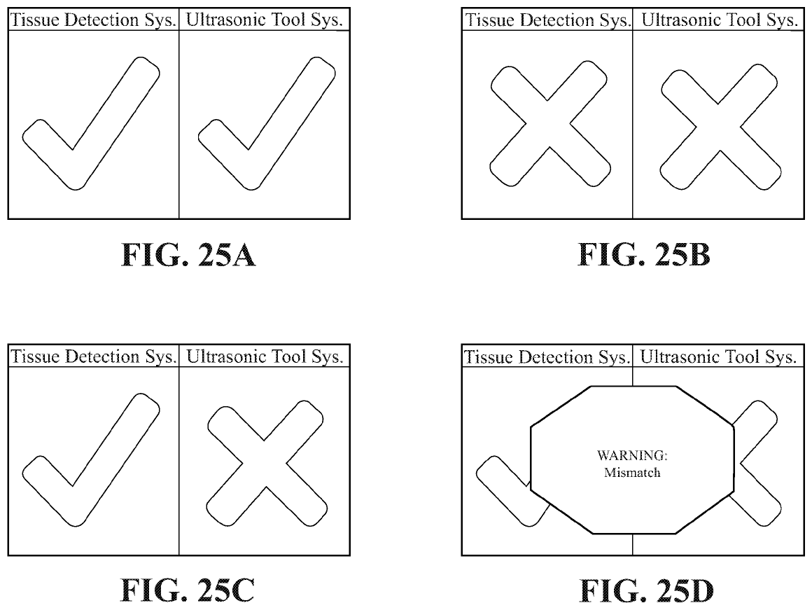

FIGS. 25A-25D illustrates a graphical user interface (GUI) that may displayed in block 814. As previously described, the tissue characteristics determined by the tissue detection system 13 and ultrasonic tool system 12 may be indicative of whether the tissue being contacted is targeted for ablation (e.g., tumorous tissue) of non-targeted (e.g., healthy tissue). Accordingly, each of the shown indicators may likewise indicate whether the tissue being contacted was determined to be targeted or non-targeted. In the illustrated examples, checkmarks provided under a given system 12, 13 may indicate that the system 12, 13 determined that the currently contacted tissue is targeted tissue, and an ‘X’ provided under a given system 12, 13 may indicate that the system 12, 13 determined that the currently contacted tissue is non-targeted tissue. FIGS. 25A and 25B each illustrate a screen that may be generated when the tissue characteristics determined by the systems 12, 13 are consistent, and FIG. 25C illustrates a screen that may be generated when the tissue characteristics determined by the systems 12, 13 differ and are thus inconsistent.

Referring again to FIG. 24, in blocks 816 and 818, the determined tissue characteristics may be compared to determine whether they are inconsistent. Responsive to determining that the tissue characteristics are inconsistent (“Yes” branch of block 818), in block 820, an error state may be triggered. In one example, the controller 112, 302 performing the comparison may be configured to trigger the error state by displaying a notification, such as illustrated in FIG. 25D. Additionally or alternatively, triggering the error state may include deactivating or pulsing the ultrasonic instrument 18 so that the practitioner may receive tactile feedback indicative of the error, and thereafter proceed with increased caution.

Responsive to an error state being triggered in block 820, or to determining that the determined tissue characteristics are not inconsistent (“No” branch of block 818), the method 400 may return to block 802 to further determine whether the ultrasonic instrument 18 is being activated, such as via the foot pedal 76, and to perform further tissue detection.

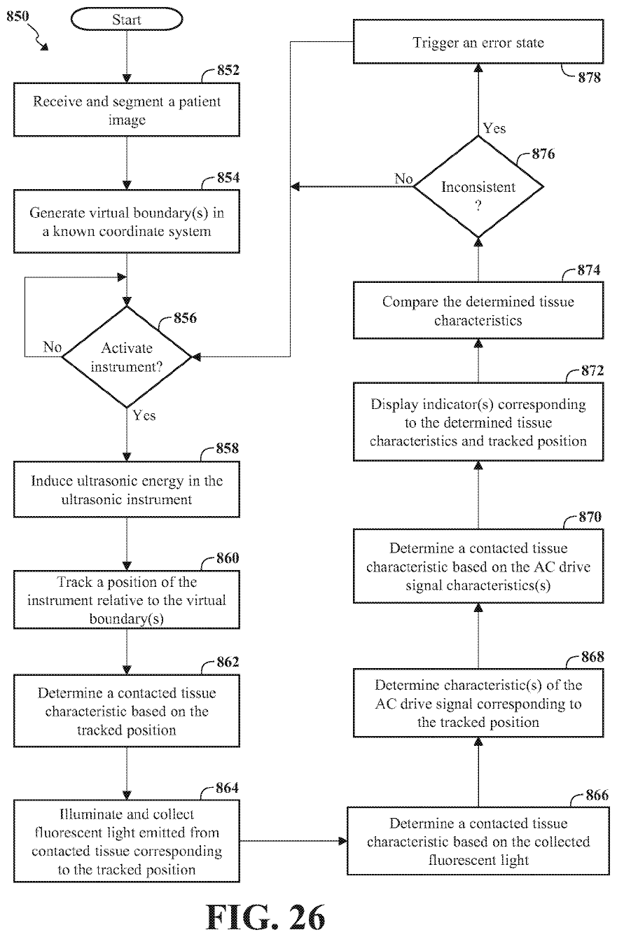

FIG. 26 illustrates a method 850 for performing tissue detection during a medical procedure with navigation. The method 850 may be implemented by the surgical control system 562, or more particularly by one or more of the controllers 112, 302, 524.

In block 852, a medical image may be received and segmented. In particular, the surgical control system 562, or more particularly the navigation controller 524 as an example, may be configured to receive a medical image including the target site TS, such as in the form of imaging data received from the imaging system 15, and to apply a segmentation algorithm to the medical image as described above to determine the position of varying tissues within the image, and correspondingly, one or more boundary(s) of varying tissues within the image.

Thereafter, in block 804, at least one virtual boundary may be generated in a known coordinate system based on the medical image. More particularly, the surgical control system 562, or more particularly the navigation controller 524 as an example, may be configured to transform the determined boundary(s) from the coordinate system specific medical image, which may correspond to that of the imaging system 15, to the localizer coordinate system LCLZ. The navigation controller 524 may perform this transformation by applying the fixed spatial relationship between the tracker 526C and the coordinate system specific to the medical image indicated by the transformation data 554 to the position and/or orientation of the imaging system tracker 526C within the localizer coordinate system LCLZ indicated by the localization data.

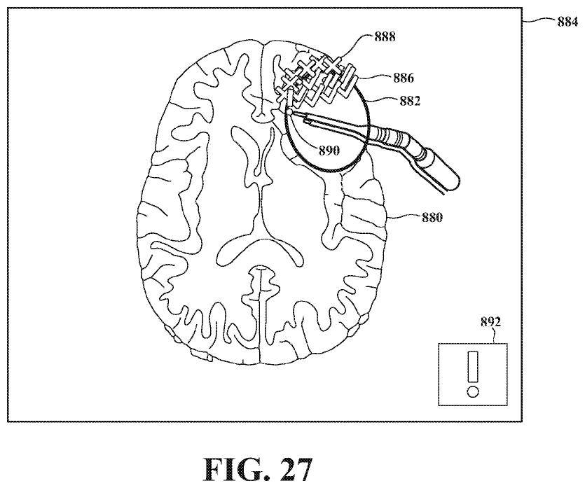

As an example, FIG. 27 illustrates a virtual boundary 880 that may be generated in the localizer coordinate system LCLZ corresponding to the periphery of a patient's brain, and another virtual boundary 882 that may be generated in association with the periphery of a target site TS of the brain, which may include tumorous tissue. In other words, the virtual boundary 882 may represent the border between non-targeted (e.g., healthy) brain tissue and targeted (e.g., tumorous) brain tissue, each type of tissue having varying characteristics relative to the AC drive signal (e.g., stiffness) and relative to fluorescence. These virtual boundary's 880, 882 may be shown as part of a GUI 884 generated by the navigation controller 524 and displayed on the user interface 532.

Referring again to FIG. 26, blocks 856 and 858 may substantially correspond to blocks 802 and 804 of FIG. 24. Following ultrasonic energy being induced in block 858, a characteristic of the tissue being contacted by the operative end 22 of the tip 20 that is indicated by localization data generated by the localizer 522 may be determined, such as based on the localization data and the at least one virtual boundary generated in the known coordinate system. More particularly, in block 860, the position and/or orientation of the ultrasonic instrument 18 relative to the virtual boundary(s) may be tracked in the known coordinate system, such as based on the localization data. More specifically, the navigation controller 524, based on the localization data, may be configured to determine a position of the operative end 22 of the tip 20 of the ultrasonic instrument 18 in the known coordinate system relative to the virtual boundary(s).

Thereafter, in block 862, a characteristic of tissue being contacted by the operative end 22 of the tip 20 may be determined based on the tracked position of the operative end 22 of the tip 20 relative to the virtual boundary(s). As previously described, each virtual boundary may be associated with one or more types of tissue. For instance, referring to FIG. 27, the internal region of the virtual boundary 882 may correspond to targeted (e.g., tumorous) brain tissue, and the region external to the virtual boundary 882 may correspond to non-targeted (e.g., healthy) brain tissue. Accordingly, based on the tracked position of the operative end 22 of the tip 20 of the ultrasonic instrument 18 relative to the virtual boundary(s) and the one or more tissue types associated with each of the virtual boundary(s), the navigation controller 524 may be configured to determine a characteristic of the tissue being contacted by the operative end 22 of the tip 20 that is indicated by the localization data.

Blocks 864 through 870 may substantially correspond to blocks 806 to 812 of FIG. 24, with each determined tissue characteristic corresponding to the tracked position and/or orientation of the ultrasonic instrument 18 determined in block 860. In other words, the position of the operative end 22 of the tip 20 determined in block 610 may correspond in time to when the fluorescent light is collected and/or when the characteristic(s) of the AC drive signal are determined. Following determination of the tissue characteristic(s), in block 872, at least one indicator corresponding to the determined tissue characteristic(s) and the tracked position and/or orientation of the ultrasonic instrument 18 may be displayed. More particularly, the surgical control system 562, or more particularly the navigation controller 524 as an example, may be configured to display a portion of the known coordinate system with at least one indicator corresponding to one or more of the determined tissue characteristics at the determined position of the operative end 22 of tip 20 of the ultrasonic instrument 18.

Thereafter, in blocks 874 and 876, the determined tissue characteristics may be compared to determine whether the there are any inconsistencies among the determined tissue characteristics. For instance, one of the controllers 112, 302, 524 may be configured to receive the tissue characteristics determined by the other controllers 112, 302. 524, and determine whether any one tissue characteristic differs from any of the other two of the received tissue characteristics. If so (“Yes” branch of block 876), in block 878, an error state may be triggered. For instance, one or more of the controllers 112, 302. 524 may be configured to trigger the error state by displaying an indication of the error, and/or by deactivating or pulsing the ultrasonic instrument 18 so that the practitioner may receive tactile feedback indicative of the error, and thereafter proceed with increased caution.

In some instances, such as responsive to the tissue characteristic determined by the navigation controller 524 differing from the tissue characteristic(s) determined by the other controllers 112, 302, the surgical control system 562, or more particularly the navigation controller 524 as an example, may be configured to adjust a position and/or orientation of the virtual boundary(s) in the known coordinate system based on the tissue characteristic(s) determined by the tissue detection system 13 and/or ultrasonic tool system 12. More specifically, based on the tissue types associated with the virtual boundary(s), the navigation controller 524 may be configured to determine a new position and/or orientation for one or more of the virtual boundary(s) such that the tissue characteristic corresponding to the latest tracked position of the operative end 22 of the tip 20 is consistent with the tissue characteristic(s) determined by the tissue detection system 13 and/or ultrasonic tool system 12, and such that one or more of the previous determined tissue characteristics corresponding to previously tracked positions of the operative end 22 of the tip 20 remain consistent with the updated position and/or orientation of the virtual boundary(s).

Additionally or alternatively, responsive to the tissue characteristic determined by the navigation controller 524 differing from the tissue characteristics determined by the other controllers 112, 302, the navigation controller 524 may be configured to adjust a position and/or orientation of the virtual boundary(s) in the known coordinate system based on updated imaging data received from the imaging system 15. Specifically, responsive to determining inconsistent tissue characteristics, the navigation controller 524 may be configured to cause the imaging system 15 to generate updated imaging data, and apply a segmentation algorithm to the updated imaging data as described above to update the positions of the virtual boundary(s). Additionally or alternatively, the navigation controller 524 may be configured to periodically receive updated image data and adjust the virtual boundary(s) if needed as described above, regardless of whether inconsistent tissue characteristics are determined.

Responsive to an error state being triggered in block 878, or to determining that the determined tissue characteristics are not inconsistent (“No” branch of block 876), the method 850 may return to block 856 to further determine whether the ultrasonic instrument 18 is being actuated, such as via the foot pedal 76, and to perform further tissue detection as described above.

FIG. 27 illustrates a graphical user interface (GUI) 884 that may be generated and displayed during the method 600, such as by the surgical control system 562, or more particularly the navigation controller 524 as an example, on the user interface 532. The GUI 884 may include at least a portion of the known coordinate system, such as the localizer coordinate system LCLZ, and may include the position and/or orientation of one or more tracked virtual boundary(s) 880, 882 in the known coordinate system.

Throughout a given procedure, in performance of the method 850, the surgical control system 562, or more particularly the navigation controller 524 as an example, may be configured to determine several positions in the known coordinate system corresponding to locations of the operative end 22 of the tip 20. For each position, one or more of the controllers 112, 302, 524 may be configured to determine one or more tissue characteristics as described above, and the surgical control system 562 may be configured to display at least one indicator at the position in the GUI 884 corresponding to the one or more determined tissue characteristics. For instance, if each of the determined tissue characteristics determined for a given position indicates that the tissue at the position is targeted (e.g., tumorous) tissue, then the GUI 884 may be configured to display a checkmark indicator 886 at the position. Alternatively, if each of the determined tissue characteristics determined for a given position indicates that the tissue at the position is non-targeted (e.g., healthy) tissue, then the GUI 884 may be configured to display an ‘X’ indicator 888 at the position. Alternatively, if the determined tissue characteristics for a given position are inconsistent, then the GUI 884 may be configured to illustrate an alert indicator 890 at the position indicative of an error. In some instances, the GUI 884 may also include a field 892 showing an enlarged view of the indicator corresponding to the currently tracked position of the operative end 22 of the tip 20 for easy viewing by the practitioner.

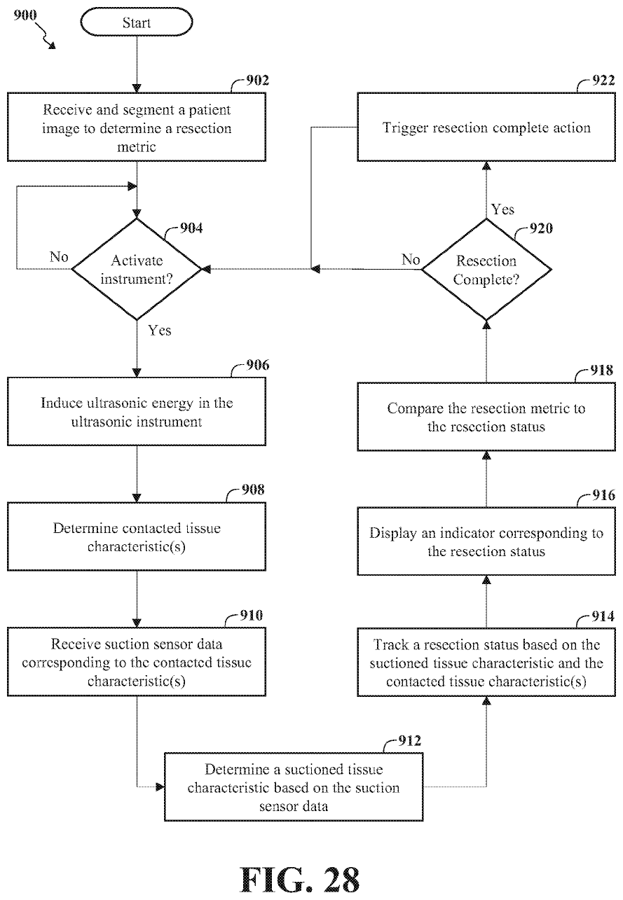

FIG. 28 illustrates a method 900 for tracking a resection status of tissue targeted for ablation during a surgical procedure. The method 900 may be implemented by the surgical control system 562, or more particularly by one or more of the controllers 112, 302, 524.

In block 902, a patient image including a target site TS with tissue targeted for ablation may be received and segmented, such as by the navigation controller 524 or the ultrasonic controller 112, to determine a resection metric. The resection metric may generally indicate a measure of the tissue targeted for ablation. For instance, the resection metric may include a resection volume, which may indicate a predetermined volume of tissue, or a predetermined volume for each of one or more types of tissue (e.g., tumorous tissue), targeted to be resected during a surgical procedure. The surgical control system 562, or more particularly the navigation controller 524 or ultrasonic controller 112, may be configured to determine the resection volume by applying a segmentation algorithm to the patient image, and thereby identify one or more boundaries corresponding to varying tissue types. The surgical control system 562, or more particularly he navigation controller 524 or ultrasonic controller 112, may be configured to thereafter determine the resection volume based on the identified boundary(s). More specifically, for each identified boundary encompassing a type of tissue targeted for resection, the surgical control system 562 may be configured to measure a volume of the tissue encompassed by the boundary within the image. In addition or alternatively, a user may be able to interact with the patient image to manually define such boundary(s) and tissue targeted for ablation within the patient image, and/or to manipulate the boundary(s) generated by the segmentation algorithm described above to further define the tissue targeted for ablation (e.g., to include a margin of healthy tissue surrounding tumorous tissue).

Additionally or alternatively, the resection metric may include a resection weight, which may indicate a predetermined weight of tissue, or a predetermined weight for each of one or more types of tissue (e.g., tumorous tissue), targeted to be resected during a surgical procedure. The resection weight may also be estimated from the medical image, such as based on the resection volume discussed above and predetermined weights associated with known tissue types.

Blocks 904 and 906 may substantially correspond to blocks 856 and 858, and to blocks 802 and 804, described above. Block 908 may substantially correspond to one or more of blocks 860 to 870, and to one or more of blocks 806 to 812, described above. Thus, following block 908, one or more contacted tissue characteristics may be determined, such as by one or more of the ultrasonic controller 112, the tissue detection controller 302, and the navigation controller 524. Such tissue characteristic(s) and the determined resection volume may then be consolidated at one of the controllers 112, 302, 524, such as the ultrasonic controller 112.

In block 910, suction sensor data corresponding to the contacted tissue characteristics(s) may be received, such as by the surgical control system 562, or more particularly ultrasonic controller 112 as an example. More specifically, as the ultrasonic instrument 18 is used to ablate and suction tissue as described above, the tissue may move through the aspiration pathway and past the suction sensor 72. As the tissue passes the suction sensor 72, the suction sensor 72 may be configured to generate suction sensor data indicative of one or more tissue characteristic(s) of the resected tissue moving through the aspiration pathway, such as a volume of the resected tissue. Additionally or alternatively, as the tissue is deposited into the waste canister 70, the weight sensor 73 may be configured to generate suction sensor data indicative of one or more tissue characteristic(s) of the resected tissue, such as a weight of the resected tissue.

In block 914, a resection status may be tracked or estimated based on the tissue characteristic(s) indicated by the suction sensor data and the contacted tissue characteristic(s) determined in block 708. To this end, the surgical control system 562, or more particularly the ultrasonic controller 112 as an example, may be configured to track a volume and/or weight of the one or more types of tissue targeted for ablation that has been resected based on the tissue characteristic(s) indicated by the suction sensor data and the determined contacted tissue characteristic(s). More specifically, the determined contacted tissue characteristic(s) may be used to indicate whether the tissue resected by the ultrasonic instrument 18, and thus moving through the aspiration pathway and into the waste canister 70, is of one of the type(s) of tissue targeted for ablation. This determination may be based at least in part on a predetermined timing criterion indicative of the time between the contacted tissue characteristic(s) being determined and the suction sensor data generated by the associated sensors corresponding to the tissue from which the contacted tissue characteristic(s) were determined. Assuming the tissue resected by the ultrasonic instrument 18 and implicated by the suction sensor data is one of the type(s) of tissue targeted for ablation, then the ultrasonic controller 112 may be configured to increase a volume tracker specific for type of tissue by the volume indicated by the suction sensor data and/or increase a weight tracker for the type of tissue by a weight indicated by the suction sensor data.

As described above, in some implementations, the suction sensor data generated by the suction sensor 72 may also indicate the type of tissue moving through the aspiration pathway. In this case, the type of tissue indicated by the suction sensor data may be used in conjunction with or alternatively to the contacted tissue characteristic(s) determined in block 908.

In block 916, the tracked resection status, or more particularly the tracked volume and/or weight for each type of tissue targeted for ablation may be displayed. The surgical control system 562, or more particularly the ultrasonic controller 112 for example, may be configured to show the tracked volume and/or weight indicated by the tracker(s) for each type of tissue targeted for ablation, such as on the display 74, and to update the same as more tissue of the type passes through the aspiration pathway.

In blocks 918 and 920, the resection status for each type of tissue targeted for ablation may be compared to the resection volume and/or weight determined for the type of tissue to determine whether resection for the type of tissue is complete. In other words, the surgical control system 562, or more particularly the ultrasonic controller 112 for example, may be configured compare the tracked volume for each type of tissue targeted for ablation to the predetermined resection volume for the type of tissue, and/or compare the tracked weight for each type of tissue targeted for ablation to the predetermined resection weight for the type of tissue. A determination may be made that resection is completed for a given type of tissue responsive to the tracked volume and/or weight for the type of tissue being greater than or equal to the predetermined resection volume and/or weight, respectively, for the given type of tissue.

Responsive to determining that resection of a given type of tissue is completed (“Yes” branch of block 920), in block 922, a resection complete action may be triggered. For instance, a notification that the type of tissue targeted for ablation has completed resection may be displayed by the surgical control system 562, such as on the display 74 of the ultrasonic tool system 12. Responsive to a resection complete action being triggered in block 922, or to determining that resection of each type of tissue targeted for ablation is not complete (“No” branch of block 920), the method 900 may return to block 904 to further determine whether to activate the ultrasonic instrument 18 and track a resection status for each type of tissue targeted for ablation, as described above.

In some implementations, the surgical control system 562, or more particularly ultrasonic controller 112 as an example, may be configured to track the resection status based on the volumes and/or weights indicated by the suction sensor data without consideration of the type of tissue, such as indicated by the contacted tissue characteristic(s) discussed above in reference to block 908. For example, the resection metric determined in block 902 may include a total resection volume and/or weight expected for all tissue targeted for ablation. Thereafter, the ultrasonic controller 112 may be configured to track a total volume and/or weight of resected tissue based on the suction sensor data, which may include subtracting from the tracked volume and/or weight a known volume and/or weight of irrigation fluid supplied to the surgical site, and comparing the result to the resection metric to determine if resection is complete. If so, then the surgical control system 562, or more particularly the ultrasonic controller 112 as an example, may be configured to trigger a resection complete action as described above.

In general, the routines executed to implement aspects of foregoing description, whether implemented as part of an operating system or a specific application, component, program, object, module or sequence of instructions, or even a subset thereof, may be referred to herein as “computer program code,” or simply “program code.” Program code may comprise computer readable instructions that are resident at various times in various memory and storage devices in a computer and that, when read and executed by one or more processors in a computer, cause that computer to perform the operations necessary to execute operations and/or elements embodying the various aspects of the description. Computer readable program instructions for carrying out operations of the various aspects of the description may be, for example, assembly language or either source code or object code written in any combination of one or more programming languages.

The program code embodied in any of the applications/modules described herein may be capable of being individually or collectively distributed as a program product in a variety of different forms. In particular, the program code may be distributed using a computer readable storage medium having computer readable program instructions thereon for causing a processor to carry out aspects of the description.

Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include random access memory (RAM), read-only memory (ROM), erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer readable storage medium should not be construed as transitory signals per se (e.g., radio waves or other propagating electromagnetic waves, electromagnetic waves propagating through a transmission media such as a waveguide, or electrical signals transmitted through a wire). Computer readable program instructions may be downloaded to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions/acts specified in the flowcharts, sequence diagrams, and/or block diagrams. The computer program instructions may be provided to one or more processors such that the instructions, which execute via the one or more processors, cause a series of computations to be performed to implement the functions and/or acts specified in the flowcharts, sequence diagrams, and/or block diagrams described herein.

In certain alternatives, the functions and/or acts described herein, such as in connection with a process or method, and/or specified in the flowcharts, sequence diagrams, and/or block diagrams may be re-ordered, processed serially, and/or processed concurrently without departing from the scope of the present disclosure. Moreover, any of the processes, methods, flowcharts, sequence diagrams, and/or block diagrams may include more or fewer blocks pr steps than those illustrated herein.

The terminology used herein is for the purpose of describing particular examples only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “includes,” “having,” “has,” “with,” “comprised of,” or variants thereof are used in either the detailed description or the claims, such terms are intended to be inclusive in a manner similar to the term “comprising.”

While a description of various examples has been provided and while these examples have been described in considerable detail, it is not the intention of the Applicant to restrict or in any way limit the scope of the appended claims to such detail. Additional advantages and modifications will readily appear to those skilled in the art. The present disclosure in its broader aspects is therefore not limited to the specific details, representative apparatus and method, and illustrative examples shown and described. Accordingly, departures may be made from such details without departing from the spirit or scope of the Applicant's general inventive concept.

Some examples are described with reference to the following numbered clauses:

Clause I. A non-transitory computer readable storage medium comprising computer-executable instructions that, upon execution by one or more processors or controllers, causes the one or more processors or controllers to: receive a medical image of a target site that includes a tumorous tissue region; based on the medical image, generate a virtual boundary associated with the tumorous tissue region in a known coordinate system; based on localization data generated by a localizer and indicative of a pose of an ultrasonic instrument in the known coordinate system, track the pose of the ultrasonic instrument in the known coordinate system; and based on the tracked pose of the ultrasonic instrument and the virtual boundary, set an AC drive signal generated by a power supply and supplied to the ultrasonic instrument to induce first pulsed ultrasonic energy in a tip of an ultrasonic instrument.

Clause II. A non-transitory computer readable storage medium comprising computer-executable instructions that, upon execution by one or more processors or controllers, causes the one or more processors or controllers to: receive a medical image of a target site that includes a first tissue region to be ablated; based on the medical image, generate a virtual boundary associated with the first tissue region in a known coordinate system; based on localization data generated by a localizer and indicative of a pose of an ultrasonic instrument in the known coordinate system, track the pose of the ultrasonic instrument in the known coordinate system; and based on the tracked pose of the ultrasonic instrument and the virtual boundary, set an AC drive signal generated by the power supply and supplied to the ultrasonic instrument to induce first pulsed ultrasonic energy in a tip of an ultrasonic instrument.

Clause III. A non-transitory computer readable storage medium comprising computer-executable instructions that, upon execution by one or more processors or controllers, causes the one or more processors or controllers to: receive a medical image of a target site that includes a soft tissue region and a hard tissue region; based on the medical image, generate a virtual boundary between the soft tissue and hard tissue regions in a known coordinate system; based on localization data generated by a localizer and indicative of a pose of an ultrasonic instrument in the known coordinate system, track the pose of the ultrasonic instrument in the known coordinate system; based on the tracked pose of the ultrasonic instrument in the known coordinate system relative to the virtual boundary, determine whether the ultrasonic instrument is within the hard tissue region or the soft tissue region; responsive to determining that the ultrasonic instrument is within the soft tissue region, generate a first AC drive signal that induces first pulsed ultrasonic energy in the ultrasonic instrument, the first pulsed ultrasonic energy comprising a plurality of first ultrasonic energy pulses interspaced by first periods of ultrasonic energy at a first minimum ultrasonic energy level, and each of the first ultrasonic energy pulses peaking at a maximum ultrasonic energy level set for the ultrasonic instrument for a second period that is less than each of the first periods; and responsive to determining that the ultrasonic instrument is within the hard tissue region, generate a second AC drive signal that induces second pulsed ultrasonic energy in the ultrasonic instrument, the second pulsed ultrasonic energy comprising a plurality of second ultrasonic energy pulses interspaced by third periods of ultrasonic energy at a second minimum ultrasonic energy level, and each of the second ultrasonic energy pulses peaking at the maximum ultrasonic energy level for a fourth period that is greater than or equal to each of the third periods.

Clause IV. A non-transitory computer readable storage medium comprising computer-executable instructions that, upon execution by one or more processors or controllers, causes the one or more processors or controllers to: receive a medical image of a target site that includes a tissue region to be ablated; based on the medical image, generate a virtual boundary associated with the tissue region; determine that localization data generated by a localizer and indicative of a pose of an ultrasonic instrument in a known coordinate system indicates a tip of the ultrasonic instrument is vibrating in the tissue region; measure one or more characteristics of an AC drive signal supplied to the ultrasonic instrument to vibrate the tip that corresponds to the localization data indicating that the tip is vibrating in the tissue region; determine that the measured one or more characteristics indicates the tip is not vibrating in the tissue region; and responsive to determining that the measured one or more characteristics indicates the tip is not vibrating in the tissue region, determine a navigation error.

Clause V. A non-transitory computer readable storage medium comprising computer-executable instructions that, upon execution by one or more processors or controllers, causes the one or more processors or controllers to: based on fluorescent light emitted from at least one fiber of a sample element coupled to an ultrasonic instrument, detect a type of tissue being contacted by a tip of the ultrasonic instrument; and based on the detected type of tissue, set an AC drive signal generated by a power supply and supplied to the ultrasonic instrument to induce first pulsed ultrasonic energy in the tip of the ultrasonic instrument.

Clause VI. A non-transitory computer readable storage medium comprising computer-executable instructions that, upon execution by one or more processors or controllers, causes the one or more processors or controllers to: determine a first tissue characteristic of tissue being contacted by the operative end of a tip of an ultrasonic instrument that is indicated by fluorescent light collected by at least one fiber of a sample element coupled to the ultrasonic instrument; determine a characteristic of an AC drive signal supplied to the ultrasonic instrument to vibrate the tip that corresponds to the collected fluorescent light indicative of the first tissue characteristic; determine a second tissue characteristic of the tissue being contacted by the operative end of the tip that is indicated by the characteristic of the AC drive signal; and display at least one indicator corresponding to the first and second tissue characteristics.

Clause VII. A non-transitory computer readable storage medium comprising computer-executable instructions that, upon execution by one or more processors or controllers, causes the one or more processors or controllers to: determine a first tissue characteristic of the tissue being contacted by the operative end of a tip of an ultrasonic instrument that is indicated by fluorescent light collected by at least one fiber of a sample element coupled to the ultrasonic instrument; determine a characteristic of an AC drive signal supplied to the ultrasonic instrument to vibrate the tip that corresponds to the collected fluorescent light indicative of the first tissue characteristic; determine a second tissue characteristic of the tissue being contacted by the operative end of the tip that is indicated by the characteristic of the AC drive signal; determine whether the first tissue characteristic is inconsistent with the second tissue characteristic; and responsive to determining that the first tissue characteristic is inconsistent with the second tissue characteristic, indicate a system error.

Clause VIII. A non-transitory computer readable storage medium comprising computer-executable instructions that, upon execution by one or more processors or controllers, causes the one or more processors or controllers to: determine a first tissue characteristic of tissue being contacted by the operative end of a tip of an ultrasonic instrument that is indicated by fluorescent light collected by at least one fiber of a sample element coupled to the ultrasonic instrument; determine a second tissue characteristic of resected tissue that moves through an aspiration pathway of the ultrasonic instrument that is indicated by a sensor coupled to the aspiration pathway; determine a resection status based on the first and second tissue characteristics; and display the resection status.

1. An ultrasonic surgical tool system comprising:
an ultrasonic instrument comprising an aspiration pathway, an irrigation pathway, a tip and a driver coupled to the tip, the driver configured to vibrate the tip to ablate tissue at a target site responsive to receiving an AC drive signal;
a power supply coupled to the ultrasonic instrument and configured to generate the AC drive signal supplied to the driver of the ultrasonic instrument;
a localizer configured to generate localization data indicative of a pose of the ultrasonic instrument in a known coordinate system; and
a control system coupled to the power supply and the localizer and configured to:
receive a medical image of the target site that includes a tumorous tissue region;
based on the medical image, generate a virtual boundary associated with the tumorous tissue region in the known coordinate system;
based on the localization data, track the pose of the ultrasonic instrument in the known coordinate system; and
based on the tracked pose of the ultrasonic instrument and virtual boundary, set the AC drive signal generated by the power supply to induce first pulsed ultrasonic energy in the tip.

2. The ultrasonic surgical tool system of claim 1, wherein the control system is configured to:
based on the tracked pose of the ultrasonic instrument in the known coordinate system relative to the virtual boundary, determine whether an operative end of the tip reaches or cross the virtual boundary from the tumorous tissue region; and
responsive to determining that the operative end of the tip reaches or cross the virtual boundary from the tumorous tissue region, set the AC drive signal generated by the power supply to induce the first pulsed ultrasonic energy in the tip.

3. The ultrasonic surgical tool system of claim 1, wherein the control system is configured to:
based on the tracked pose of the ultrasonic instrument in the known coordinate system relative to the virtual boundary, determine whether an operative end of the tip is within the tumorous tissue region; and
responsive to determining that the operative end of the tip is within the tumorous tissue region, set the AC drive signal generated by the power supply to induce continuous ultrasonic energy in the tip.

4. The ultrasonic surgical tool system of claim 1, wherein the first pulsed ultrasonic energy includes a plurality of first ultrasonic energy pulses interspaced by first periods of ultrasonic energy at a first minimum ultrasonic energy level, each of the first ultrasonic energy pulses peaking at a maximum ultrasonic energy level set for the ultrasonic instrument for a second period that is less than each of the first periods.

5. The ultrasonic surgical tool system of claim 1, wherein the first pulsed ultrasonic energy includes a plurality of first ultrasonic energy pulses interspaced by ultrasonic energy at a first minimum ultrasonic energy level, each of the first ultrasonic energy pulses peaks at a maximum ultrasonic energy level set for the ultrasonic instrument, and the first minimum ultrasonic energy level is less than or equal to 5% of the maximum ultrasonic energy level.

6. The ultrasonic surgical tool system of claim 1, wherein the control system is configured to:
based on the tracked pose of the ultrasonic instrument in the known coordinate system relative to the virtual boundary, determine whether an operative end of the tip is within the tumorous tissue region with a distance between the operative end of the tip and the virtual boundary being less than a first threshold distance; and
responsive to determining that the operative end of the tip is within the tumorous tissue region with the distance between the operative end of the tip and the virtual boundary being less than the first threshold distance, set the AC drive signal generated by the power supply to induce the first pulsed ultrasonic energy in the tip.

7. The ultrasonic surgical tool system of claim 6, wherein the first pulsed ultrasonic energy includes a plurality of first ultrasonic energy pulses interspaced by first periods of ultrasonic energy at a first minimum ultrasonic energy level, each of the first ultrasonic energy pulses peaking at a maximum ultrasonic energy level set for the ultrasonic instrument for a second period that is less than each of the first periods.

8. The ultrasonic surgical tool system of claim 6, wherein the first pulsed ultrasonic energy includes a plurality of first ultrasonic energy pulses interspaced by ultrasonic energy at a first minimum ultrasonic energy level, each of the first ultrasonic energy pulses peaks at a maximum ultrasonic energy level set for the ultrasonic instrument, and the first minimum ultrasonic energy level is greater than or equal to 10% of the maximum ultrasonic energy level.

9. The ultrasonic surgical tool system of claim 6, wherein the control system is configured to:
based on the tracked pose of the ultrasonic instrument in the known coordinate system relative to the virtual boundary, determine whether the operative end of the tip is within the tumorous tissue region with the distance between the operative end of the tip and the virtual boundary being greater than the first threshold distance; and
responsive to determining that the operative end of the tip is within the tumorous tissue region with the distance between the operative end of the tip and the virtual boundary being greater than the first threshold distance, set the AC drive signal generated by the power supply to induce continuous ultrasonic energy in the tip.

10. The ultrasonic surgical tool system of claim 6, wherein the first pulsed ultrasonic energy is generated based on a first modulation waveform, and the control system is configured to:
based on the tracked pose of the ultrasonic instrument in the known coordinate system relative to the virtual boundary, determine whether the operative end of the tip is within the tumorous tissue region with the distance between the operative end of the tip and the virtual boundary being greater than the first threshold distance; and
responsive to determining that the operative end of the tip is within the tumorous tissue region with the distance between the operative end of the tip and the virtual boundary being greater than the first threshold distance, set the AC drive signal generated by the power supply to induce second pulsed ultrasonic energy in the tip, the second pulsed ultrasonic energy being generated based on a second modulation waveform that differs from the first modulation waveform.

11. The ultrasonic surgical tool system of claim 6, wherein the first pulsed ultrasonic energy is generated based on a first modulation waveform, and the control system is configured to:
based on the tracked pose of the ultrasonic instrument in the known coordinate system relative to the virtual boundary, determine whether the operative end of the tip is within the tumorous tissue region with the distance between the operative end of the tip and the virtual boundary being greater than the first threshold distance and less than a second threshold distance; and
responsive to determining that the operative end of the tip is within the tumorous tissue region with the distance between the operative end of the tip and the virtual boundary being greater than the first threshold distance and less than the second threshold distance, set the AC drive signal generated by the power supply to induce second pulsed ultrasonic energy in the tip, the second pulsed ultrasonic energy being generated based on a second modulation waveform that differs from the first modulation waveform.

12. The ultrasonic surgical tool system of claim 11, wherein the control system is configured to:
based on the tracked pose of the ultrasonic instrument in the known coordinate system relative to the virtual boundary, determine whether the operative end of the tip is within the tumorous tissue region with the distance between the operative end of the tip and the virtual boundary being greater than the second threshold distance; and
responsive to determining that the operative end of the tip is within the tumorous tissue region to be removed with the distance between the operative end of the tip and the virtual boundary being greater than the second threshold distance, set the AC drive signal generated by the power supply to induce continuous ultrasonic energy in the tip.

13. The ultrasonic surgical tool system of claim 10, wherein the first pulsed ultrasonic energy includes a plurality of first ultrasonic energy pulses interspaced by ultrasonic energy at a first minimum ultrasonic energy level, the second pulsed ultrasonic energy includes a plurality of second ultrasonic energy pulses interspaced by ultrasonic energy at a second minimum ultrasonic energy level, and the first minimum ultrasonic energy level is less than the second minimum ultrasonic energy level.

14. The ultrasonic surgical tool system of claim 13, wherein each of the first ultrasonic energy pulses and each of the second ultrasonic energy pulses peak at a maximum ultrasonic energy level set for the ultrasonic instrument.

15. The ultrasonic surgical tool system of claim 14, wherein first minimum ultrasonic energy level is greater than 10% of the maximum ultrasonic energy level, and the second minimum ultrasonic energy level is greater than 20% of the maximum ultrasonic energy level.

16. The ultrasonic surgical tool system of claim 14, wherein the first ultrasonic energy pulses are interspaced by first periods of ultrasonic energy at the first minimum ultrasonic energy level and each peaks at the maximum ultrasonic energy level for a second period that is less than each of the first periods, the second ultrasonic energy pulses are interspaced by third periods of ultrasonic energy at the second minimum ultrasonic energy level and each peaks at the maximum ultrasonic energy level for a fourth period that is less than each of the third periods, and each of the first periods is greater than each of the third periods.

17. The ultrasonic surgical tool system of claim 10, wherein the first pulsed ultrasonic energy has a first pulsing frequency, and the second pulsed ultrasonic energy has a second pulsing frequency that is less than the first pulsing frequency.

18. The ultrasonic surgical tool system of claim 10, wherein the first pulsed ultrasonic energy has a first duty cycle and the second pulsed ultrasonic energy has a second duty cycle that is greater than the first duty cycle.

19. The ultrasonic surgical tool system of claim 6, wherein the first pulsed ultrasonic energy is generated based on a first modulation waveform, and the control system is configured to:
based on the tracked pose of the ultrasonic instrument in the known coordinate system, determine whether an operative end of the tip reaches or crosses the virtual boundary from the tumorous tissue region; and
responsive to determining that the operative end of the tip reaches or crosses the virtual boundary from the tumorous tissue region, set the AC drive signal generated by the power supply to induce third pulsed ultrasonic energy in the tip, the third pulsed ultrasonic energy being generated based on a modulation waveform that differs from the first pulsed ultrasonic energy.

20. The ultrasonic surgical tool system of claim 19, wherein the third pulsed ultrasonic energy includes a plurality of third ultrasonic energy pulses interspaced by ultrasonic energy at a third minimum ultrasonic energy level and each peaking at a maximum ultrasonic energy level set for the ultrasonic instrument, the third minimum ultrasonic energy level being less than or equal to 5% of the maximum ultrasonic energy level.

21.-117. (canceled)
